(19) United States
(12) Patent Application Publication
(10) Pub. No.: US 2025/0051805 A1
(43) Pub. Date: Feb. 13, 2025

(54) METHODS

(71) Applicant: Commonwealth Scientific and Industrial Research Organisation, Acton (AU)

(72) Inventors: Michael Anthony AYLIFFE, Acton (AU); Thomas David Loan, Acton (AU); Ming Luo, Acton (AU)

(73) Assignee: Commonwealth Scientific and Industrial Research Organisation, Acton (AU)

(21) Appl. No.: 18/572,581

(22) PCT Filed: Jun. 24, 2022
(86) PCT No.: PCT/AU2022/050653

§ 371 Date: Dec. 20, 2023

(30) Foreign Application Priority Data
Jun. 25, 2021 (AU) 2021901926

(51) Int. Cl. C12P 5/02; A61K 31/02

(52) U.S. Cl. CPC C12P 5/02; A61K 31/02

(57) ABSTRACT

The present invention relates to methods of increasing bromoform production.

### CROSS-REFERENCE TO RELATED APPLICATIONS

This application is a § 371 national stage of PCT International Application No. PCT/AU2022/050653, filed Jun. 24, 2022, claiming priority of Australian Application No. 2021901926, filed Jun. 25, 2021, the entire contents of each of which are hereby incorporated by reference into the subject application.

### REFERENCE TO SEQUENCE LISTING

This application incorporates-by-reference nucleotide and/or amino acid sequences which are present in the file named “241031_92358_SequenceListing_DH.txt”, which is 247,037 bytes in size, and which was created on Oct. 28, 2024 in the IBM-PC machine format, having an operating system compatibility with MS-Windows, which is contained in the text file filed Oct. 31, 2024 as part of this application.

### FIELD OF THE INVENTION

The present invention relates to methods of increasing bromoform production.

### BACKGROUND OF THE INVENTION

Vanadate-dependent haloperoxidases (VHPOs) are enzymes found in prokaryotic and eucaryotic organisms including bacteria, cyanobacteria, fungi, marine algae and lichens. These enzymes use hydrogen peroxide to oxidase a halide anion (Cl⁻, Br⁻, I⁻) to form hypohalous acid (X—OH), which diffuse into solution and react with numerous substrates as an electrophile (Franssen et al., 1988). Di- and tri-containing halide compounds are formed by VHPO catalysed reactions on a range of organic compounds including 3-oxo-octanoic acid, oxalate, phosphenylpyruvate, pyruvate etc. (Ohsawa et al., 2001).

As shown in FIG. 1, repeated bromination of ketones followed by hydrolysis (haloform reaction) is thought to be responsible for bromoform formation. Pathways for bromoform production by Cyanobacteria have been investigated by Thapa et al. (2020) and Thapa and Agarwal (2021). Beta-ketoacid substrates like 3-oxooctanoic acid have been shown to produce bromoform (Theiler et al., 1978) using enzymes purified from the marine red algae, Bonnemaisonia hamifera.

Of note is the VHPO-catalysed production of bromoform (CHBr₃) by marine algae, in particular Asparagopsis taxiformis and Asparagopsis armata, and the observation that these algae produce sufficient bromoform to inhibit methanogenesis in ruminant livestock upon feeding. This could dramatically reduce livestock methane emissions, which are a significant contributor to global greenhouse gases (Machado et al., 2016).

Genes encoding VHPO enzymes have been cloned from a number of algal and bacterial species and shown to produce bromoform under appropriate conditions when introduced into Escherichia coli, Saccharomyces cerevisiae and Pichia pastoris. However, bromoform levels are generally low in many organisms with endogenous VHPO and recombinant systems, in part due to the unknown nature of the organic intermediate(s) required for optimal bromoform production. Thus, there is a need for methods of increasing bromoform production, particularly from recombinant microbes expressing recombinant VHPOs.

### SUMMARY OF THE INVENTION

The present inventors have identified methods for increasing bromoform production.

In one aspect, the present invention provides a method of producing bromoform, the method comprising incubating an organism or part thereof, cells, a lysate of the organism or part thereof or cells, or a mixture thereof, comprising a vanadate-dependent haloperoxidase, in the presence of at least one compound of Formula 1:

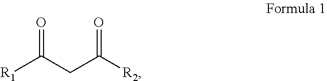

wherein R₁ and R₂ are:
independently selected from: hydrogen, hydroxyl, optionally substituted aliphatic, optionally substituted O-alkyl, or optionally substituted S-alkyl, or
R₁ and R₂ are joined to form an optionally substituted six membered ring of Formula 1a:

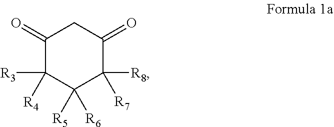

wherein R₃, R₄, R₅, R₆, R₇, and R₈ are each independently selected from hydrogen or an optionally substituted aliphatic.

In an embodiment, the method further comprises incubating the organism, part thereof or cells thereof in the presence of at least one or more or all of:
(i) a catalase activity inhibitor;
(ii) catalase gene(s) modification-;
(iii) a compound that promotes the accumulation of acetoacyl-acyl-carrier-protein;
(iv) a FabG or FAS gene modification;
(v) conditions that promote β-oxidation.

In an embodiment, the compound that promotes the accumulation of acetoacyl-acyl-carrier-proteins is a FabG or FAS inhibitor, preferably tannic acid.

In an embodiment, the conditions that promote β-oxidation comprise incubating the organism, part thereof or cells thereof in the presence of a fatty acid as a carbon source, preferably a medium chain or long chain fatty acid. In another embodiment the fatty acid is oleic acid.

In an embodiment, the catalase activity inhibitor is 3-amino-1,2,4-triazol. In another embodiment, the catalase activity inhibitor is selected from the group consisting of 2,2′-azino-bis(3-ethylbenzthiazoline-6-sulfonic acid), 3-amino-1,2,4-triazole, 3-amino-4-hydroxybenzoic acid, azide, Ba²⁺, Co²⁺, Cu²⁺, EDTA, H₂O₂, KCl, MgCl₂, NaCl and nicotinic acid hydrazide.

In another embodiment, the catalase gene(s) modification is a knock-down or knock-out deletion of both cytosolic and peroxisomal catalase genes.

In an embodiment, the vanadate-dependent haloperoxidase is a vanadium chloroperoxidase (VCPO) or a vanadium bromoperoxidase (VBPO).

In an embodiment, the organism is a microorganism and the method comprises culturing the microorganism.

In an embodiment, the method comprises culturing the cells. In an embodiment, the cells are eukaryote cells such as plant cells or animal cells. In an embodiment, the cells are plant cells.

In an embodiment, the organism is a plant, and the method comprises growing the plant. In an embodiment, the part is a vegetative part of a plant such as a leaf or stem. In an embodiment, the plant is an angiosperm. In an embodiment, the plant is a macroalgae.

In another aspect, the present invention provides a method of producing bromoform, the method comprising incubating conditioned medium obtained from culturing a microorganism, cells, a lysate of the microorganism or cells, or a mixture thereof, comprising a vanadate-dependent haloperoxidase, in the presence of at least one compound of Formula 1:

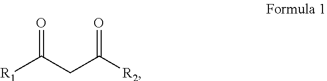

wherein R₁ and R₂ are:
independently selected from: hydrogen, hydroxyl, optionally substituted aliphatic, optionally substituted O-alkyl, or optionally substituted S-alkyl, or
R₁ and R₂ are joined to form an optionally substituted six membered ring of Formula 1a:

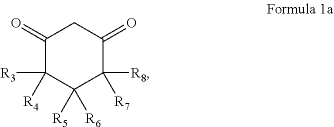

wherein R₃, R₄, R₅, R₆, R₇, and R₈ are each independently selected from hydrogen or an optionally substituted aliphatic, and wherein the conditioned medium comprises the vanadate-dependent haloperoxidase.

In an embodiment, the method further comprises incubating the conditioned medium obtained from culturing a microorganism, cells, a lysate of the microorganism or cells, or a mixture thereof in the presence of at least one or more or all of:
(i) a catalase activity inhibitor;
(ii) a compound that promotes the accumulation of acetoacyl-acyl-carrier-proteins;
   optionally wherein the conditioned medium was obtained from culturing a microorganism, cells, or a mixture thereof comprising a catalase gene(s) modification and/or a FabG or FAS gene modification, and/or conditions that promote β-oxidation.

Examples of microorganisms useful for the invention include, but are not limited to, a bacteria, fungi and algae.

In an embodiment, the algae is a microalgae.

In an embodiment, the fungi is yeast or a filamentous fungi.

Examples of yeast suitable for the useful for the invention include, but are not limited to, those selected from the group of Genera consisting of Arxula, Candida, Ogataea, Kluyveromyces, Pichia, Saccharomyces, and Yarrowia. In an embodiment, the yeast is Saccharomyces cerevisiae, Yarrowia lipolytica or Pichia pastoris.

Examples of filamentous fungi useful for the invention include, but are not limited to, those selected from the group of Genera consisting of Alternaria, Curvularia, Drechslera, Bipolaris, Ulocladium, Botrytis (such as such as Botrytis cinerea), Fusarium, Penicillium and Aspergillus (such as Aspergillus oryzae).

Examples of bacteria useful for the invention include, but are not limited to, Cyanobacteria, Bacillus subtilis or Escherichia coli.

In an embodiment, the organism, microorganism or cells are non-viable.

In an embodiment, the organism naturally comprises the vanadate-dependent haloperoxidase. In an alternate embodiment, the organism does not naturally comprise the vanadate-dependent haloperoxidase.

In an embodiment, the method further comprises harvesting the organism, microorganism or cells.

In an embodiment, the method further comprises, following culturing, harvesting the medium.

In another aspect, the present invention provides a method of producing bromoform, the method comprising incubating a vanadate-dependent haloperoxidase in the presence of at least one compound of Formula 1:

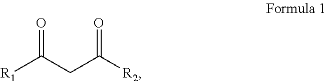

wherein R₁ and R₂ are:
independently selected from: hydrogen, hydroxyl, optionally substituted aliphatic, optionally substituted O-alkyl, or optionally substituted S-alkyl, or
R₁ and R₂ are joined to form an optionally substituted six membered ring of Formula 1a:

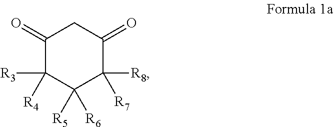

wherein R₃, R₄, R₅, R₆, R₇, and R₈ are each independently selected from hydrogen or an optionally substituted aliphatic, and wherein one or more or all of the following apply:
i) the vanadate-dependent haloperoxidase is not present in a protein extract obtained from an organism which naturally produces the vanadate-dependent haloperoxidase,
ii) the vanadate-dependent haloperoxidase is incubated with the at least one compound for greater than 90 minutes,
iii) the incubating occurs in the presence of hydrogen peroxide, and if hydrogen peroxide is added more than once, then at least two of the additions are greater than 10 minutes apart.

In an embodiment, the method further comprises incubating the vanadate-dependent haloperoxidase in the presence of at least one or more or all of:
(i) a catalase activity inhibitor;
(ii) a microorganism, cells, or a mixture thereof comprising a catalase gene(s) modification;
(iii) a compound that promotes the accumulation of acetoacyl-acyl-carrier-proteins;
(iv) a microorganism, cells, or a mixture thereof comprising a FabG or FAS gene modification;
(v) a microorganism, cells, or a mixture thereof cultured under conditions that promote β-oxidation.

In another aspect, the present invention provides a method of producing bromoform, the method comprising incubating a vanadate-dependent haloperoxidase in the presence of at least one compound of Formula 1:

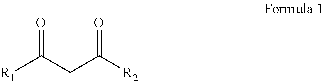

wherein: R₁ and R₂ are:
independently selected from: hydrogen, hydroxyl, optionally substituted aliphatic, optionally substituted O-alkyl; optionally substituted S-alkyl, with the proviso that the compound is not oxaloacetic acid nor acetylacetone, or
R₁ and R₂ are joined to form an optionally substituted six membered ring of Formula 1a:

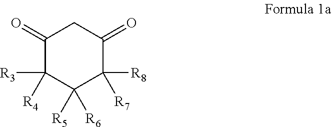

wherein R₃, R₄, R₅, R₆, R₇, and R₈ are each independently selected from hydrogen or an optionally substituted aliphatic.

In an embodiment, the method further comprises incubating the vanadate-dependent haloperoxidase in the presence of at least one or more or all of:
(i) a catalase activity inhibitor;
(ii) a microorganism, cells, or a mixture thereof comprising a catalase gene(s) modification;
(iii) a compound that promotes the accumulation of acetoacyl-acyl-carrier-proteins;
(iv) a microorganism, cells, or a mixture thereof comprising a FabG or FAS gene modification;
(v) a microorganism, cells, or a mixture thereof cultured under conditions that promote β-oxidation.

In an embodiment of the two above aspect, the vanadate-dependent haloperoxidase is present in, and/or produced by, an organism.

In an embodiment, the at least one compound has a pKa of about 11 or less, about 10 or less, about 9 or less between about 4 and about 12, or between about 5 and about 10.7.

In an embodiment, the at least one compound is 5,5-dimethyl-1,3-cyclohexanedione, acetylacetone, 3,5-heptanedione, ethyl acetoacetate, S-ethyl acetothioacetate, acetoacetyl coenzyme A, or a mixture of any two or more or all thereof. In an embodiment, the compound is acetylacetone. In an embodiment the compound is 5,5-dimethyl-1,3-cyclohexanedione. In an embodiment the compound is acetoacetyl coenzyme A.

In an embodiment, the at least one compound is 5,5-dimethyl-1,3-cyclohexanedione, 3,5-heptanedione, ethyl acetoacetate, acetoacetyl coenzyme A, S-ethyl acetothioacetate, or a mixture of any two or more or all thereof.

In an embodiment, the concentration of the at least one compound in the medium is at least 4 mM, at least 5 mM, at least 10 mM, between 4 mM and 20 mM, between 4 mM and 15 mM or between 4 mM and 10 mM.

In an embodiment, the method produces at least 5, at least 6, at least 7, at least 8, or at least 9 fold more bromoform than a method performed under the same conditions in the absence of the at least one compound of Formula 1.

In another embodiment, a method of the invention produces at least 5, at least 10, at least 15, at least 20, at least 25, at least 30, at least 35, at least 40, at least 45 or at least 50 fold more bromoform than a method performed under the same conditions in the absence of least one compound of Formula 1 and/or in the absence of at least one or more or all of:
(i) a catalase activity inhibitor;
(ii) an organism or part thereof, microorganism or cells comprising a catalase gene(s) modification;
(iii) a compound that promotes the accumulation of acetoacyl-acyl-carrier-proteins;
(iv) an organism or part thereof, microorganism or cells comprising a FabG or FAS gene modification;
(v) conditions that promote β-oxidation.

In an embodiment, the organism or part thereof, microorganism or cells comprise an exogenous polynucleotide encoding the vanadate-dependent haloperoxidase.

In an embodiment, the vanadate-dependent haloperoxidase comprises a sequence of amino acids provided in any one of SEQ ID NO's 1 to 8, or a sequence which is at least 50%, at least 60%, at least 70%, at least 80%, at least 90% or at least 95% identical to any one or more or all of SEQ ID NO's 1 to 8. In an embodiment, the vanadate-dependent haloperoxidase comprises a sequence of amino acids provided in SEQ ID NO:1 or SEQ ID NO:2.

In an embodiment, the polynucleotide comprises a sequence of nucleotides provided in any one of SEQ ID NO's 9 to 19, or a sequence which is at least 50%, at least 60%, at least 70%, at least 80%, at least 90% or at least 95% identical to any one or more or all of SEQ ID NO's 9 to 19.

In an embodiment, the exogenous polynucleotide encoding the vanadate-dependent haloperoxidase is operably linked to a sequence encoding a peroxisome targeting signal (PTS). An example of a peroxisome targeting signal includes peroxisome targeting signal 1 which is a group of C-terminal signals (PTS1) typically ending with amino acids SKL. An example of this type of PTS is ePTS1 tag, according to the nucleotide sequence defined in SEQ ID NO: 22. Another example of a suitable PTS is PTS2, which is a less conversed set of peptides near the N-terminus. Such PTSs have the consensus sequence R-(L/V/I/Q-X2-(L/V/I/H)-(L/S/G/A)-X-(H/Q)-(L/A).

In another embodiment, the exogenous polynucleotide encoding the vanadate-dependent haloperoxidase is operably linked to a constitutive promoter for expression in an organism or part thereof, microorganism or cells.

In a further embodiment, there is provided a yeast for producing bromoform, the yeast comprising an exogenous polynucleotide encoding the vanadate-dependent haloperoxidase, wherein the yeast comprises one or more catalase gene modifications and/or is incubated in the presence of a catalase activity inhibitor. Preferably, the exogenous polynucleotide encoding the vanadate-dependent haloperoxidase is operably linked to a sequence encoding a peroxisome targeting signal (PTS). In an embodiment, the yeast is selected the group consisting of Arxula, Candida, Ogataea, Kluyveromyces, Pichia, Saccharomyces, Yarrowia. Preferably the yeast is selected from the group consisting of Saccharomyces cerevisiae, Yarrowia lipolytica or Pichia pastoris.

In an embodiment, the culturing is conducted in a closed system and the bromoform is captured at least in the headspace of the system.

In an embodiment, the culturing is for between 1 hour and 24 hours, at least 2 hours or at least 3 hours.

Also provided is bromoform produced using a method of the invention.

Also provided is conditioned medium comprising bromoform obtained using a method of the invention. In an embodiment, the conditioned medium comprises a low amount of glucose, for instance, 1%, 1.5% or 2% glucose. In an embodiment, the range of glucose used in the media is between about 0.5-2.5%, between about 1.0-2.5%, between about 1.5-2.5%, between about 2.0-2.5%, between about 0.5-2.0%, between about 0.5-1.5%, or between about 0.5-1.0%.

In another aspect, the present invention provides an extract or lysate of an organism or part thereof, microorganism or cells incubated in accordance with a method of the invention, wherein the extract or lysate comprises bromoform.

In another aspect, the present invention provides a feedstuff, drink or animal feed supplement comprising one or more or all of bromoform produced using a method of the invention, the conditioned (culture) medium of the invention or the extract or lysate of the invention, and with at least one other feed, drink or supplement ingredient.

In another aspect, the present invention provides a composition comprising one or more or all of bromoform produced using a method of the invention, the conditioned (culture) medium of the invention or the extract or lysate of the invention, and comprising at least one feed, drink or supplement ingredient.

In another aspect, the present invention provides a method of producing a feedstuff, drink or animal supplement, the method comprising combining one or more or all of bromoform produced using a method of the invention, the culture medium of the invention or the extract or lysate of the invention, with at least feed, drink or supplement ingredient.

In another aspect, the present invention provides a method of feeding an animal, the method comprising providing the animal with a feedstuff, drink or animal supplement of the invention.

The animal may be a ruminant or a non-ruminant. In an embodiment, the animal is a ruminant such as a cow, sheep, goat, deer or camel.

In an embodiment, the method of the above aspect reduces methane production by the animal.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1: Enzymatic (1) and non-enzymatic (2 and 3) steps involved in bromoform production.

FIG. 2: Deletion of the catalase coding regions by PCR.

FIG. 3: Calibration curve. Bromoform was quantified by comparison of the signal corresponding to the molecular ion (m/z=249±0.5) with a standard curve prepared from authentic samples of bromoform in brine in the same way as the samples to account for any matrix effects.

FIG. 4: Relative production of bromoform with E. coli expressing the A. marina VHPO gene relative to the E. coli only control.

FIG. 5: Relative concentrations of bromoform and dibromoacetone in E. coli culture samples.

FIG. 6: Mechanism for bromoform synthesis from acetylacetone.

FIG. 7: A. Bromoform production with E. coli cells with addition of 400 mM H₂O₂ indicated by the arrows at 0, 120 and 240 minutes. B. Calibration curve used to measure bromoform concentrations in this example.

FIG. 8: Relative bromoform production with E. coli expressing the A. marina VHPO gene relative to the E. coli only control comparing a range of 1,3 dicarbonyl containing substrates.

FIG. 9: Bromoform production with S. cerevisiae expressing the A. marina or A. taxiformis VHPO genes comparing a range of substrates.

FIG. 10: Bromoform production from different substrates with purified enzyme.

FIG. 11: A. Comparison of VHPO activity in control (top) with E. coli expressing VHPO (bottom). Image was taken three minutes after resuspending cells in 200 μL of assay mixture (100 mM HEPES (pH 7.4) 20 mM KBr, 1 mM Na₃VO₄, 100 mM H₂O₂). Bubbles visible in the samples are the result of catalase present in the cells converting H₂O₂ to O₂ gas. B. Production of bromoform with the addition of IPTG, KBr, Na₃VO₄ and amino-triazole to inhibit catalase.

FIG. 12: A. Comparison of VHPO activity in BY4741ΔCTT1ΔCTA1-pRSVHPO-ePST grown in Sc-U+2% glucose (top) and Sc-U+10% glycerol+0.1% oleic acid (bottom). Cells from 100 μL culture samples were harvested by centrifugation, resuspended in assay buffer 200 μL of assay mixture (100 mM HEPES (pH 7.4) 20 mM KBr, 1 mM Na₃VO₄, 10 mM H₂O₂) and left overnight to visualize activity. Note that because these cells carry no catalase genes, a lower H₂O₂ concentration was used. B. GC-MS data, extracted ion chromatogram and associated mass spectra corresponding to bromoform standard (Top) and for S. cerevisiae grown in the presence of oleic acid (Bottom).

### KEY TO THE SEQUENCE LISTING

SEQ ID NO:1—Acaryochloris marina vanadium-dependent haloperoxidase

SEQ ID NO:2—Asparagopsis taxiformis vanadium-dependent haloperoxidase

SEQ ID NO:3—Ascophyllum nodosum vanadium-dependent haloperoxidase

SEQ ID NO:4—Chondrus crispus vanadium-dependent haloperoxidase

SEQ ID NO:5—Corallina pilubfera vanadium-dependent bromoperoxidase

SEQ ID NO:6—Luminaria digitata vanadium-dependent haloperoxidase

SEQ ID NO:7—Curvularia inaequalis vanadium-dependent haloperoxidase

SEQ ID NO:8—Open reading frame encoding Alternaria arborescens vanadium dependent haloperoxidase

SEQ ID NO:9—Open reading frame encoding Acaryochloris marina vanadium-dependent haloperoxidase

SEQ ID NO:10—Open reading frame encoding Asparagopsis taxiformis vanadium-dependent haloperoxidase

SEQ ID NO:11—Open reading frame encoding Ascophyllum nodosum vanadium-dependent haloperoxidase

SEQ ID NO:12—Open reading frame encoding Chondrus crispus vanadium-dependent haloperoxidase

SEQ ID NO:13—Open reading frame encoding Corallina pilulifera vanadium-dependent bromoperoxidase

SEQ ID NO:14—Open reading frame encoding Luminaria digitata vanadium-dependent haloperoxidase

SEQ ID NO:15—Open reading frame encoding Curvularia inaequalis vanadium-dependent haloperoxidase

SEQ ID NO:16—Open reading frame encoding Alternaria arborescens vanadium dependent haloperoxidase

SEQ ID NO:17—Open reading frame encoding Acaryochloris marina vanadium-dependent haloperoxidase codon optimised for expression in E. coli

SEQ ID NO:18—Open reading frame encoding Acaryochloris marina vanadium-dependent haloperoxidase codon optimised for expression in S. cerevisiae

SEQ ID NO:19—Open reading frame encoding Acaryochloris marina vanadium-dependent haloperoxidase codon optimised for expression in P. pastoris

SEQ ID NO's 20 and 21—Oligonucleotide primers

SEQ ID NO:22—Nucleotide sequence of ePST1 tag for localisation to the peroxisome

SEQ ID NO's 23-46—Primer sequences used for CTT1 deletion, CTA1 deletion, site directed mutagenesis, pRS-ePST1VHPO assembly and pRS-ePST1VHPO sequencing (Table 2).

SEQ ID NO:47—Nucleotide sequence encoding CTA1-URA3

SEQ ID NO:48—Nucleotide sequence encoding CTT1-URA3

SEQ ID NO:49—Nucleotide sequence encoding pACYCDuet-1-VBPO

SEQ ID NO:50—Nucleotide sequence encoding pCRISPYL-34265gRNA1

SEQ ID NO:51—Nucleotide sequence encoding pCRISPYL-34265gRNA2

SEQ ID NO:52—Nucleotide sequence encoding pCRISPYL-34749gRNA1

SEQ ID NO:53—Nucleotide sequence encoding pCRISPYL-34749gRNA2

SEQ ID NO:54—Nucleotide sequence encoding pCRISPYL-CTA1

SEQ ID NO:55—Nucleotide sequence encoding pRS-pTef2-AmVHPO-SKL

SEQ ID NO:56—Nucleotide sequence encoding pYES2-AmVHPO-SKL

SEQ ID NO:57—Nucleotide sequence encoding pYLHR-34265gRNA1

SEQ ID NO:58—Nucleotide sequence encoding pYLHR34265gRNA2

SEQ ID NO:59—Nucleotide sequence encoding pYLHR34749gRNA2

SEQ ID NO:60—Nucleotide sequence encoding pYLHR34749RNA1

SEQ ID NO:61—Nucleotide sequence encoding pYLHR-CTA::AmVHPOSKL

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, bromoform production and use, molecular genetics, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−10%, more preferably +/−5%, more preferably +/−1%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, a “lysate” refers to microorganisms or cells that have been lysed. Cells that are lysed are no longer intact, and hence have broken cell membranes and/or cell walls, or may only have fragments of cell membranes and/or cell walls. Cell lysates may be prepared using standard methods, for example, by mechanical means (e.g., shearing or crushing) or chemical means (e.g., using a detergent). In an embodiment, the lysed cells have not been fractionated to separate out any cell components. In an embodiment, the cell lysate has been fractionated and the supernatant taken for use in the invention. In an embodiment, at least some of the microorganisms or cells become lysed during the culturing process. In an embodiment, the lysate is of a part of the organism.

As used herein, “non-viable” organism, microorganism or cells means they are no longer viable and hence are unable to reproduce or divide. Non-viable organisms, microorganisms or cells are intact in the sense they have not been lysed. Non-viable organisms, microorganisms or cells can be obtained by a variety of means known in the art such as freeze/thawing, chemical or heat treatment.

As used herein, a “protein extract” refers to a sample obtained from an organism (such as a microorganism) or part thereof or cells, that has been subjected to processing to enrich the level of protein, for example by at least 50%, at least 75% or at least 90%.

As used herein, “conditioned media” or “conditioned medium” refers to media that has been used for the incubation of an enzymatic reaction, and/or for culturing an organism (such as a microorganism) or part thereof or cells, and comprises products of the reaction, organism (such as a microorganism) or part thereof or cells. In an embodiment, “conditioned media” or “conditioned medium” of the invention comprises a vanadate-dependent haloperoxidase, bromoform, or both.

Herein “aliphatic” refers to an alkyl, alkenyl, alkynyl, or carbocyclyl group, as defined.

“Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include optionally substituted: methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl.

As used herein, “alkylene,” “alkenylene,” and “alkynylene,” refer to a divalent radical of an alkyl, alkenyl, and alkynyl group, respectively. When a range or number of carbons is provided for a particular “alkylene,” “alkenylene,” and “alkynylene” group, it is understood that the range or number refers to the range or number of carbons in the linear carbon divalent chain. “Alkylene,” “alkenylene,” and “alkynylene” groups may be substituted or unsubstituted with one or more substituents as described herein.

“Alkylene” refers to an alkyl group wherein two hydrogens are removed to provide a divalent radical, and which may be substituted or unsubstituted. Unsubstituted alkylene groups include, but are not limited to: methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted alkylene groups, e.g., substituted with one or more alkyl (methyl) groups, include but are not limited to, substituted methylene (—CH(CH₃)—, (—C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), and the like.

“Alkenyl” refers to a radical of a straight-chain or branched hydrocarbon group having from 2 to 20 carbon atoms, one or more carbon-carbon double bonds (e.g., 1, 2, 3, or 4 carbon-carbon double bonds). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

Herein “alkenylene” refers to an alkenyl group wherein two hydrogens are removed to provide a divalent radical, and which may be substituted or unsubstituted. Exemplary unsubstituted divalent alkenylene groups include, but are not limited to, ethenylene (—CH═CH—) and propenylene (e.g., —CH═CHCH₂—, —CH₂—CH═CH—). Exemplary substituted alkenylene groups, e.g., substituted with one or more alkyl (methyl) groups, include but are not limited to, substituted ethylene (—C(CH₃)═CH—, —CH═C(CH₃)—), substituted propylene (e.g., —C(CH₃)=CHCH₂—, —CH═C(CH₃)CH₂—, —CH═CHCH(CH₃)—, —CH═CHC(CH₃)₂—, —CH(CH₃)—CH═CH—, —C(CH)₂—CH═CH—, —CH₂—C(CH₃)=CH—, —CH₂— CH═C(CH₃)—), and the like.

Herein “alkynyl” refers to a radical of a straight-chain or branched hydrocarbon group having from 2 to 20 carbon atoms, one or more carbon-carbon triple bonds, e.g., 1, 2, 3, or 4 carbon-carbon triple bonds, (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

Herein “alkynylene” refers to a linear alkynyl group wherein two hydrogens are removed to provide a divalent radical, and which may be substituted or unsubstituted. Exemplary divalent alkynylene groups include, but are not limited to, substituted or unsubstituted ethynylene, substituted or unsubstituted propynylene, and the like.

Herein “carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

“Halo” or “halogen” refers to fluoro (F), chloro (Cl), bromo (Br), and iodo (I).

The terms “optionally substituted”, “comprises one or more substituents” or “substituted” means that a corresponding radical, atom, group or moiety on a compound may have one or more substituents present. Where a plurality of substituents, or a selection of various substituents is specified, the substituents are selected independently of one another and do not need to be identical. In some cases, at least one hydrogen atom on the radical, group or moiety is replaced with a substituent. In the case of an oxo substituent (=O) two hydrogen atoms may be replaced. In this regard, substituents may include one or more: alkyl, alkenyl, alkynyl, carbocyclyl, halogen, nitro, cyano, hydroxy, sulfonic, thiol, ether, amino, alkylamino, dialkylamino, haloalkyl, hydroxyalkyl, alkoxy, haloalkoxy, aryloxy, heteroaryloxy, aralkyloxy, alkylthio, carboxamido, sulfonamido, alkylcarbonyl, arylcarbonyl, alkylsulfonyl, arylsulfonyl, carboxy, carboxyalkyl, alkyl, cycloalkyl, alkenyl, alkynyl, aryl, heteroaryl, heterocyclo, alkoxyalkyl, (amino)alkyl, hydroxyalkylamino, (alkylamino)alkyl, (dialkylamino)alkyl, (cyano)alkyl, (carboxamido)alkyl, mercaptoalkyl, (heterocyclo)alkyl, (cycloalkylamino)alkyl, (C₁-C₄ haloalkoxy)alkyl, (heteroaryl)alkyl, or perylene, oxo, heterocycle, —OR^{x}, —NR^{x}R^{Y}, —NR^{x}C(═O)R^{y}—NR^{x}SO₂R^{y}, —C(═O)R^{x}, —C(═O)OR^{x}, —C(═O)NR^{x}R^{y}, —SO_{q}R^{x}, —SO_{q}NR^{x}R^{y}, and mixtures thereof, wherein q is 0, 1 or 2, R^{x} and R^{y} are the same or different and independently selected from hydrogen, alkyl or heterocycle, and each of said alkyl and heterocycle substituents may be further substituted with one or more of oxo, halogen, —OH, —CN, alkyl, —OR^{x}, heterocycle, —NR^{x}R^{y}, —NR^{x}C(═O)R^{y}—NR^{x}SO₂R^{y}, —C(═O)R^{x}, —C(═O)OR^{x}, —C(═O)NR^{x}R^{y}, —SOR^{x} and —SONR^{x}R^{y}. In one embodiment, the terms “optionally substituted”, “comprises one or more substituents” or “substituted” also comprise a substituent of Formula 3:

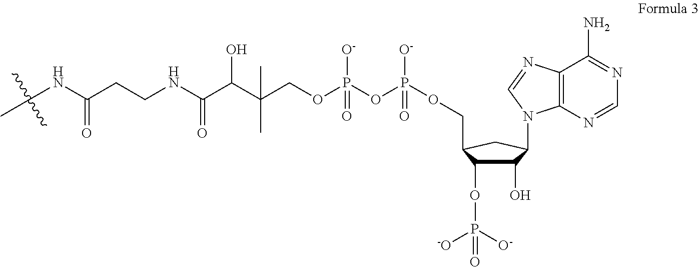

wherein [symbol] is the point of attachment for the substituent of Formula 3.

### Vanadate-Dependent Haloperoxidase

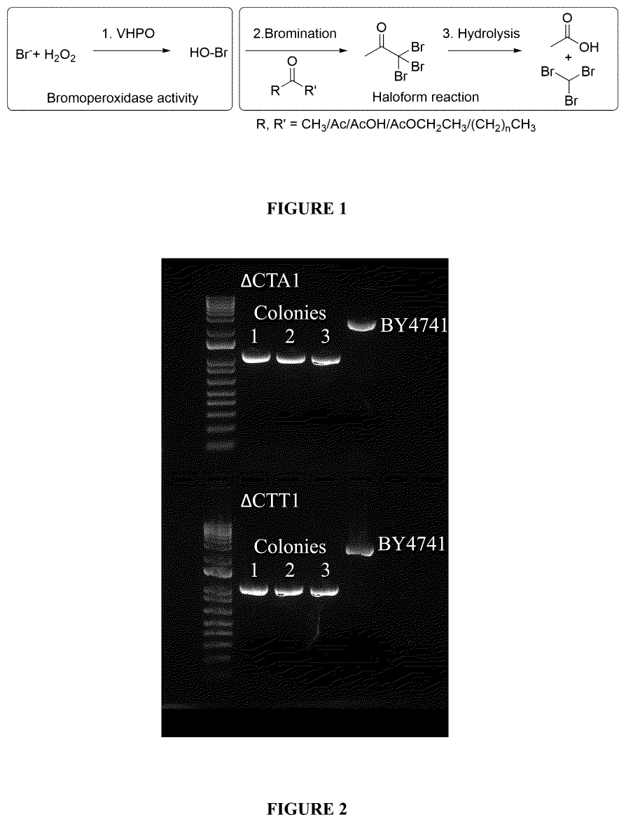

As used herein, “vanadate-dependent haloperoxidase” or “VHPO” refers to an enzyme that contains a vanadate prosthetic group and utilize hydrogen peroxide to oxidize a halide ion into a reactive electrophilic intermediate. Such VHPO include vanadium chloroperoxidase (EC 1.11.1.10) which are capable of oxidising chloride, bromide and iodide and bromoperoxidases (EC 1.11.1.18) which catalyse the oxidation of bromide and iodide. Vanadate-dependent haloperoxidase useful for the invention have bromoperoxidase activity, and hence are able to convert bromide ions (Br⁻) into Br—OH in the presence of hydrogen peroxide (FIG. 1). Examples of VHPO's useful for the invention include, but are not limited to, those from red, brown and green algae. In an embodiment, the VHPO is from species of the Genus Corallina, Bonnemaisonia, Asparagopsis (such as Asparagopsis taxiformis), Ascophyllum (such as Ascophyllum nodosum) or Acaryochloris (such as Acaryochloris marina). In an embodiment the VHPO is from a soil fungi species of the Genus Curvularia (such as Curvularia inaequalis) Alternaria (such as Alternaria didymospora), Fusarium, Drechslera, Bipolaris, Ulocladium, Aspergillus (such as Aspergillus oryzae) or Botrytis (such as Botrytis cinerea).

Further suitable examples of vanadate-dependent haloperoxidases from terrestrial species are defined in U.S. Pat. No. 4,707,447, which in incorporated in its entirety by reference, and include Fusarium (such as Fusarium oxysporum), Drechslera (such as Drechslera subpapendorfii or halodes), Bipolaris, Ulocladium, Ulocladium chartarum, Aspergillus (such as Aspergillus niger), and plant pathogens such as Magnaporte grisea and Phaeosphaeria nodorum.

In an embodiment, the vanadate-dependent haloperoxidase comprises a sequence of amino acids provided in any one of SEQ ID NO's 1 to 8, or a sequence which is at least 50%, at least 60%, at least 70%, at least 80%, at least 90% or at least 95% identical to any one or more or all of SEQ ID NO's 1 to 8.

In an embodiment, the VHPO may be provided in the form of an extracted enzyme. In this embodiment, when used in the presence of dimidone, bromoform yield may be increased by up to about 50 fold, when compared to the effect of VHPO in the presence of a control e.g., acetone.

The terms “polypeptide” and “protein” are generally used interchangeably.

The % identity of a polypeptide is determined by GAP (Needleman and Wunsch, 1970) analysis (GCG program) with a gap creation penalty=5, and a gap extension penalty=0.3. The query sequence is at least 500 amino acids in length, and the GAP analysis aligns the two sequences over a region of at least 500 amino acids. More preferably, the query sequence is at least 600 amino acids in length and the GAP analysis aligns the two sequences over a region of at least 6000 amino acids. Even more preferably, the GAP analysis aligns two sequences over their entire length.

With regard to a defined polypeptide, it will be appreciated that % identity figures higher than those provided above will encompass preferred embodiments. Thus, where applicable, in light of the minimum % identity figures, it is preferred that the polypeptide comprises an amino acid sequence which is preferably at least 60%, at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

Amino acid sequence mutants of the polypeptides disclosed herein can be prepared by introducing appropriate nucleotide changes into a nucleic acid defined herein, or by in vitro synthesis of the desired polypeptide. Such mutants include, for example, deletions, insertions or substitutions of residues within the amino acid sequence. A combination of deletion, insertion and substitution can be made to arrive at the final construct, provided that the final product possesses the desired characteristics, namely vanadate-dependent haloperoxidase activity. Preferred amino acid sequence mutants have one, two, three, four or less than 10 amino acid changes relative to the reference wildtype polypeptide.

Mutant (altered) polypeptides can be prepared using any technique known in the art, for example, using directed evolution, rational design strategies or mutagenesis (see below). Products derived from mutated/altered DNA can readily be screened using techniques described herein to determine if, when expressed in, for example yeast, produce Br—OH in the presence of at least one compound of Formula 1 and Br—.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide molecule removed and a different residue inserted in its place. Where it is desirable to maintain a certain activity it is preferable to make no, or only conservative substitutions, at amino acid positions which are highly conserved in the relevant protein family. Examples of conservative substitutions are shown in Table 1 under the heading of “exemplary substitutions”.

TABLE 1
Exemplary substitutions.

| Original Residue | Exemplary Substitutions |
|---|---|
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe, ala |

In a preferred embodiment, a mutant/variant polypeptide has one or two or three or four conservative amino acid changes when compared to a naturally occurring polypeptide. Details of conservative amino acid changes are provided in Table 1. In a preferred embodiment, the changes are not in one or more of the motifs which are highly conserved between the different polypeptides provided herewith, and/or not in the important motifs of vanadate-dependent haloperoxidase polypeptides. As the skilled person would be aware, such minor changes can reasonably be predicted not to alter the activity of the polypeptide when expressed in a recombinant cell.

The primary amino acid sequence of a polypeptide of the invention can be used to design variants/mutants thereof based on comparisons with closely related polypeptides. As the skilled addressee will appreciate, residues highly conserved amongst closely related proteins are less likely to be suitable to be altered, especially with non-conservative substitutions, and activity maintained than less conserved residues (see above).

In an embodiment, the polynucleotide encodes a vanadate-dependent haloperoxidase comprising a peroxisomal targeting signal such as described in WO 2020/243792. In an embodiment, the peroxisomal targeting signal is at the C-terminal end of the vanadate-dependent haloperoxidase.

### Compounds for Increasing Bromoform Production

In accordance with the invention at least one compound of Formula 1:

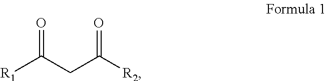

is used in the culturing of an organism to produce bromoform.
R₁ and R₂ are:
independently selected from: hydrogen, hydroxyl, optionally substituted aliphatic, optionally substituted O-alkyl, or optionally substituted S-alkyl; or
R₁ and R₂ are joined to form an optionally substituted six membered ring of Formula 1a:

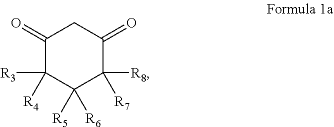

wherein R₃, R₄, R₅, R₆, R₇, and R₈ are each independently selected from hydrogen or an optionally substituted aliphatic.

In one embodiment R₁ and R₂ are the same.

In one embodiment R₁ and R₂ are different.

In one embodiment at least one of R₁ and R₂ is hydrogen.

In one embodiment at least one of R₁ and R₂ is an optionally substituted aliphatic.

In one embodiment at least one of R₁ and R₂ is an optionally substituted group selected from: alkyl, alkenyl, alkynyl and carbocyclyl.

In one embodiment at least one of R₁ and R₂ is OH.

In one embodiment at least one of R₁ and R₂ is an optionally substituted alkyl group. In another embodiment at least one of R₁ and R₂ is an optionally substituted: C₁₋₂₀ alkyl, C₁₋₁₂ alkyl, C₁₋₁₀ alky, C₁₋₉ alkyl, C₁₋₈ alkyl, C₁₋₇ alkyl, C₁₋₆ alkyl, C₁₋₅ alkyl, C₁₋₄ alkyl, C₁₋₃ alkyl, C₁₋₂ alkyl, C₁ alkyl, or C₂₋₆ alkyl. In yet another embodiment at least one of R₁ and R₂ is an optionally substituted alkyl group selected from: methyl, ethyl, n-propyl, isopropyl, n-butyl, tert-butyl, sec-butyl, iso-butyl, n-pentyl, 3-pentanyl, amyl, neopentyl, 3-methyl-2-butanyl, tertiary amyl, and n-hexyl, n-heptyl, and n-octyl.

In one embodiment both R₁ and R₂ are optionally substituted alkyl groups independently selected from: C₁₋₂₀ alkyl, C₁₋₁₂ alkyl, C₁₋₁₀ alky, C₁₋₉ alkyl, C₁₋₈ alkyl, C₁₋₇ alkyl, C₁₋₆ alkyl, C₁₋₅ alkyl, C₁₋₄ alkyl, C₁₋₃ alkyl, C₁₋₂ alkyl, C₁ alkyl, and C₂₋₆ alkyl. In another embodiment R₁ and R₂ is the same substituted or unsubstituted alkyl group selected from: C₁₋₂₀ alkyl, C₁₋₁₂ alkyl, C₁₋₁₀ alky, C₁₋₉ alkyl, C₁₋₈ alkyl, C₁₋₇ alkyl, C₁₋₆ alkyl, C₁₋₅ alkyl, C₁₋₄ alkyl, C₁₋₃ alkyl, C₁₋₂ alkyl, C₁ alkyl, and C₂₋₆ alkyl. In yet another embodiment both R₁ and R₂ are unsubstituted alkyl groups independently selected from: C₁₋₂₀ alkyl, C₁₋₁₂ alkyl, C₁₋₁₀ alky, C₁₋₉ alkyl, C₁₋₈ alkyl, C₁₋₇ alkyl, C₁₋₆ alkyl, C₁₋₅ alkyl, C₁₋₄ alkyl, C₁₋₃ alkyl, C₁₋₂ alkyl, C₁ alkyl, and C₂₋₆ alkyl.

In one embodiment at least one of R₁ and R₂ is an optionally substituted methyl group. In one embodiment at least one of R₁ and R₂ is an optionally substituted ethyl group.

In one embodiment at least one of R₁ and R₂ is an optionally substituted alkenyl group. In one embodiment at least one of R₁ and R₂ is an optionally substituted: C₂₋₁₀ alkenyl, C₂₋₉ alkenyl, C₂₋₈ alkenyl, C₂₋₇ alkenyl, C₂₋₆ alkenyl, C₂₋₅ alkenyl, C₂₋₄ alkenyl, C₂₋₃ alkenyl, or C₂ alkenyl. In yet another embodiment at least one of R₁ and R₂ is an optionally substituted alkenyl group selected from: ethenyl, 1-propenyl, 2-propenyl, 1-butenyl, 2-butenyl, butadienyl, pentenyl, pentadienyl, hexenyl, heptenyl, octenyl, and octatrienyl.

In one embodiment at least one of R₁ and R₂ is an optionally substituted alkynyl group. In another embodiment at least one of R₁ and R₂ is an optionally substituted: C₂₋₂₀ alkynyl, C₂₋₁₀ alkynyl, C₂₋₉ alkynyl, C₂₋₈ alkynyl, C₂₋₇ alkynyl, C₂₋₆ alkynyl, C₂₋₅ alkynyl, C₂₋₄ alkynyl, C₂₋₃ alkynyl, or C₂ alkynyl. In yet another embodiment at least one of R₁ and R₂ is an optionally substituted alkynyl group selected from: ethynyl, 1-propynyl, 2-propynyl, 1-butynyl, 2-butynyl, pentynyl, hexynyl, heptynyl, and octynyl.

In one embodiment at least one of R₁ and R₂ is an optionally substituted carbocyclyl group. In another embodiment at least one of R₁ and R₂ is an optionally substituted: C₃₋₁₀ carbocyclyl, C₃₋₈ carbocyclyl, C₃₋₆ carbocyclyl, or C₅₋₁₀ carbocyclyl. In yet another embodiment at least one of R₁ and R₂ is an optionally substituted carbocyclyl group selected from: cyclopropyl, cyclopropenyl, cyclobutyl, cyclobutenyl, cyclopentyl, cyclopentenyl, cyclohexyl, cyclohexenyl, cyclohexadienyl, cycloheptyl, cycloheptenyl, cycloheptadienyl, cycloheptatrienyl, cyclooctyl, cyclooctenyl, bicyclo[2.2.1]heptanyl, bicyclo[2.2.2]octanyl, cyclononyl, cyclononenyl, cyclodecyl, cyclodecenyl, octahydro-1H-indenyl, decahydronaphthalenyl, and spiro[4.5]decanyl.

In one embodiment at least one of R₁ and R₂ is an optionally substituted O-alkyl group. In another embodiment at least one of R₁ and R₂ is an O-alkyl group comprising an optionally substituted: C₁₋₂₀ alkyl, C₁₋₁₂ alkyl, C₁₋₁₀ alky, C₁₋₉ alkyl, C₁₋₈ alkyl, C₁₋₇ alkyl, C₁₋₆ alkyl, C₁₋₅ alkyl, C₁₋₄ alkyl, C₁₋₃ alkyl, C₁₋₂ alkyl, C₁ alkyl, or C₂₋₆ alkyl. In yet another embodiment at least one of R₁ and R₂ is an O-alkyl group grouping comprising an optionally substituted alkyl group selected from: methyl, ethyl, n-propyl, isopropyl, n-butyl, tert-butyl, sec-butyl, iso-butyl, n-pentyl, 3-pentanyl, amyl, neopentyl, 3-methyl-2-butanyl, tertiary amyl, and n-hexyl, n-heptyl, and n-octyl.

In one embodiment:
one of R₁ and R₂ is an O-alkyl group comprising an optionally substituted: C₁₋₂₀ alkyl, C₁₋₁₂ alkyl, C₁₋₁₀ alky, C₁₋₉ alkyl, C₁₋₈ alkyl, C₁₋₇ alkyl, C₁₋₆ alkyl, C₁₋₅ alkyl, C₁₋₄ alkyl, C₁₋₃ alkyl, C₁₋₂ alkyl, C₁ alkyl, or C₂₋₆ alkyl; and
one of R₁ and R₂ is an optionally substituted alkyl group selected from: C₁₋₂₀ alkyl, C₁₋₁₂ alkyl, C₁₋₁₀ alky, C₁₋₉ alkyl, C₁₋₈ alkyl, C₁₋₇ alkyl, C₁₋₆ alkyl, C₁₋₅ alkyl, C₁₋₄ alkyl, C₁₋₃ alkyl, C₁₋₂ alkyl, C₁ alkyl, and C₂₋₆ alkyl.

In one embodiment:
one of R₁ and R₂ is an O-alkyl group comprising an optionally substituted: C₁₋₅ alkyl, C₁₋₄ alkyl, C₁₋₃ alkyl, C₁₋₂ alkyl, or C₁ alkyl; and
one of R₁ and R₂ is an optionally substituted alkyl group selected from: C₁₋₅ alkyl, C₁₋₄ alkyl, C₁₋₃ alkyl, C₁₋₂ alkyl, and C₁ alkyl.

In one embodiment at least one of R₁ and R₂ is an optionally substituted S-alkyl group.

In another embodiment at least one of R₁ and R₂ is a S-alkyl group comprising an optionally substituted: C₁₋₂₀ alkyl, C₁₋₁₂ alkyl, C₁₋₁₀ alky, C₁₋₉ alkyl, C₁₋₈ alkyl, C₁₋₇ alkyl, C₁₋₆ alkyl, C₁₋₅ alkyl, C₁₋₄ alkyl, C₁₋₃ alkyl, C₁₋₂ alkyl, C₁ alkyl, or C₂₋₆ alkyl. In yet another embodiment at least one of R₁ and R₂ is a S-alkyl group grouping comprising an optionally substituted alkyl group selected from: methyl, ethyl, n-propyl, isopropyl, n-butyl, tert-butyl, sec-butyl, iso-butyl, n-pentyl, 3-pentanyl, amyl, neopentyl, 3-methyl-2-butanyl, tertiary amyl, and n-hexyl, n-heptyl, and n-octyl.

In another embodiment, at least one of R₁ and R₂ is a S-alkyl group comprising a substituent of Formula 3.

In one embodiment:
one of R₁ and R₂ is a S-alkyl group comprising an optionally substituted: C₁₋₂₀ alkyl, C₁₋₁₂ alkyl, C₁₋₁₀ alky, C₁₋₉ alkyl, C₁₋₈ alkyl, C₁₋₇ alkyl, C₁₋₆ alkyl, C₁₋₅ alkyl, C₁₋₄ alkyl, C₁₋₃ alkyl, C₁₋₂ alkyl, C₁ alkyl, or C₂₋₆ alkyl; and
one of R₁ and R₂ is an optionally substituted alkyl group selected from: C₁₋₂₀ alkyl, C₁₋₁₂ alkyl, C₁₋₁₀ alky, C₁₋₉ alkyl, C₁₋₈ alkyl, C₁₋₇ alkyl, C₁₋₆ alkyl, C₁₋₅ alkyl, C₁₋₄ alkyl, C₁₋₃ alkyl, C₁₋₂ alkyl, C₁ alkyl, and C₂₋₆ alkyl.

In one embodiment:
one of R₁ and R₂ is a S-alkyl group comprising an optionally substituted: C₁₋₅ alkyl, C₁₋₄ alkyl, C₁₋₃ alkyl, C₁₋₂ alkyl, or C₁ alkyl; and
one of R₁ and R₂ is an optionally substituted alkyl group selected from: C₁₋₅ alkyl, C₁₋₄ alkyl, C₁₋₃ alkyl, C₁₋₂ alkyl, and C₁ alkyl.

In one embodiment:
one of R₁ and R₂ is a S-alkyl group comprising a substituent of Formula 3, wherein the alkyl group is selected from: C₁₋₅ alkyl, C₁₋₄ alkyl, C₁₋₃ alkyl, C₁₋₂ alkyl, or C₁ alkyl; and
one of R₁ and R₂ is an optionally substituted alkyl group selected from: C₁₋₅ alkyl, C₁₋₄ alkyl, C₁₋₃ alkyl, C₁₋₂ alkyl, and C₁ alkyl.

In one embodiment, R₃, R₄, R₅, R₆, R₇, and R₈ are each independently selected from hydrogen or an optionally substituted aliphatic.

In one embodiment at least: 2, 3, 4, 5 or 6, of R₃, R₄, R₅, R₆, R₇ and R₈, are the same.

In one embodiment at least one of R₃, R₄, R₅, R₆, R₇ and R₈ is hydrogen. In another embodiment, 1, 2, 3, 4, 5, or 6 of R₃, R₄, R₅, R₆, R₇ and R₈, is/are hydrogen.

In one embodiment at least one of R₃, R₄, R₅, R₆, R₇ and R₈ is an optionally substituted aliphatic. In one embodiment at least one of R₃, R₄, R₅, R₆, R₇ and R₈ is an unsubstituted aliphatic. In another embodiment at least one of R₃, R₄, R₅, R₆, R₇ and R₈ is a substituted aliphatic. In yet another embodiment, 1, 2, 3, 4, 5, or 6 of R₃, R₄, R₅, R₆, R₇ and R₈, is/are optionally substituted aliphatic.

In one embodiment at least one of R₃, R₄, R₅, R₆, R₇ and R₈ is an optionally substituted group selected from: alkyl, alkenyl, alkynyl and carbocyclyl.

In one embodiment at least one of R₃, R₄, R₅, R₆, R₇ and R₈ is an optionally substituted alkyl group. In another embodiment, 1, 2, 3, 4, 5, or 6 of R₃, R₄, R₅, R₆, R₇ and R₈, is/are optionally substituted alkyl. In another embodiment at least one of R₃, R₄, R₅, R₆, R₇ and R₈ is an optionally substituted: C₁₋₂₀ alkyl, C₁₋₁₂ alkyl, C₁₋₁₀ alky, C₁₋₉ alkyl, C₁₋₈ alkyl, C₁₋₇ alkyl, C₁₋₆ alkyl, C₁₋₅ alkyl, C₁₋₄ alkyl, C₁₋₃ alkyl, C₁₋₂ alkyl, C₁ alkyl, or C₂₋₆ alkyl. In yet another embodiment at least one of R₃, R₄, R₅, R₆, R₇ and R₈ is an optionally substituted alkyl group selected from: methyl, ethyl, n-propyl, isopropyl, n-butyl, tert-butyl, sec-butyl, iso-butyl, n-pentyl, 3-pentanyl, amyl, neopentyl, 3-methyl-2-butanyl, tertiary amyl, and n-hexyl, n-heptyl, and n-octyl.

In one embodiment at least one of R₃, R₄, R₅, R₆, R₇ and R₈ is an optionally substituted alkenyl group. In another embodiment, 1, 2, 3, 4, 5, or 6 of R₃, R₄, R₅, R₆, R₇ and R₈, is/are optionally substituted alkenyl. In one embodiment at least one of R₃, R₄, R₅, R₆, R₇ and R₈ is an optionally substituted: C₂₋₁₀ alkenyl, C₂₋₉ alkenyl, C₂₋₈ alkenyl, C₂₋₇ alkenyl, C₂₋₆ alkenyl, C₂₋₅ alkenyl, C₂₋₄ alkenyl, C₂₋₃ alkenyl, or C₂ alkenyl. In yet another embodiment at least one of R₃, R₄, R₅, R₆, R₇ and R₈ is an optionally substituted alkenyl group selected from: ethenyl, 1-propenyl, 2-propenyl, 1-butenyl, 2-butenyl, butadienyl, pentenyl, pentadienyl, hexenyl, heptenyl, octenyl, and octatrienyl.

In one embodiment at least one of R₃, R₄, R₅, R₆, R₇ and R₈ is an optionally substituted alkynyl group. In another embodiment, 1, 2, 3, 4, 5, or 6 of R₃, R₄, R₅, R₆, R₇ and R₈, is/are optionally substituted alkynyl. In another embodiment at least one of R₃, R₄, R₅, R₆, R₇ and R₈ is an optionally substituted: C₂₋₂₀ alkynyl, C₂₋₁₀ alkynyl, C₂₋₉ alkynyl, C₂₋ₛ alkynyl, C₂₋₇ alkynyl, C₂₋₆ alkynyl, C₂₋₅ alkynyl, C₂₋₄ alkynyl, C₂₋₃ alkynyl, or C₂ alkynyl. In yet another embodiment at least one of R₃, R₄, R₅, R₆, R₇ and R₈ is an optionally substituted alkynyl group selected from: ethynyl, 1-propynyl, 2-propynyl, 1-butynyl, 2-butynyl, pentynyl, hexynyl, heptynyl, and octynyl.

In one embodiment at least one of R₃, R₄, R₅, R₆, R₇ and R₈ is an optionally substituted carbocyclyl group. In another embodiment, 1, 2, 3, 4, 5, or 6 of R₃, R₄, R₅, R₆, R₇ and R₈, is/are optionally substituted carbocyclyl. In another embodiment at least one of R₃, R₄, R₅, R₆, R₇ and R₈ is an optionally substituted: C₃₋₁₀ carbocyclyl, C₃₋₈ carbocyclyl, C₃₋₆ carbocyclyl, or C₅₋₁₀ carbocyclyl. In yet another embodiment at least one of R₃, R₄, R₅, R₆, R₇ and R₈ is an optionally substituted carbocyclyl group selected from: cyclopropyl, cyclopropenyl, cyclobutyl, cyclobutenyl, cyclopentyl, cyclopentenyl, cyclohexyl, cyclohexenyl, cyclohexadienyl, cycloheptyl, cycloheptenyl, cycloheptadienyl, cycloheptatrienyl, cyclooctyl, cyclooctenyl, bicyclo[2.2.1]heptanyl, bicyclo[2.2.2]octanyl, cyclononyl, cyclononenyl, cyclodecyl, cyclodecenyl, octahydro-1H-indenyl, decahydronaphthalenyl, and spiro[4.5]decanyl.

In another embodiment:
one or two of R₃, R₄, R₅, R₆, R₇ and R₈ is an optionally substituted: C₁₋₂₀ alkyl, C₁₋₁₂ alkyl, C₁₋₁₀ alky, C₁₋₉ alkyl, C₁₋₈ alkyl, C₁₋₇ alkyl, C₁₋₆ alkyl, C₁₋₅ alkyl, C₁₋₄ alkyl, C₁₋₃ alkyl, C₁₋₂ alkyl, C₁ alkyl, or C₂₋₆ alkyl; and
four or five of R₃, R₄, R₅, R₆, R₇ and R₈ is hydrogen.

In another embodiment, at least one of R₅ and R₆ is an optionally substituted: C₁₋₅ alkyl, C₁₋₄ alkyl, C₁₋₃ alkyl, C₁₋₂ alkyl, or C₁ alkyl. In yet another embodiment each of R₅ and R₆ is an optionally substituted: C₁₋₅ alkyl, C₁₋₄ alkyl, C₁₋₃ alkyl, C₁₋₂ alkyl, or C₁ alkyl.

In one embodiment, four of R₃, R₄, R₅, R₆, R₇ and R₈ are hydrogen, and two of of R₃, R₄, R₅, R₆, R₇ and R₈ are optionally substituted: C₁₋₅ alkyl, C₁₋₄ alkyl, C₁₋₃ alkyl, C₁₋₂ alkyl, or C₁ alkyl. For example, R₃, R₄, R₇ and R₈ may be hydrogen, and R₅ and R₆, may be optionally substituted: C₁₋₅ alkyl, C₁₋₄ alkyl, C₁₋₃ alkyl, C₁₋₂ alkyl, or C₁ alkyl.

In one embodiment the at least one compound of Formula 1 is a compound of Formula 2:

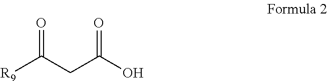

wherein: R₉ is selected from: optionally substituted aliphatic, or optionally substituted O-alkyl.

In one embodiment R₉ is an optionally substituted alkyl group. In another embodiment R₉ is an optionally substituted: C₁₋₂₀ alkyl, C₁₋₁₂ alkyl, C₁₋₁₀ alky, C₁₋₉ alkyl, C₁₋₈ alkyl, C₁₋₇ alkyl, C₁₋₆ alkyl, C₁₋₅ alkyl, C₁₋₄ alkyl, C₁₋₃ alkyl, C₁₋₂ alkyl, C₁ alkyl, or C₂₋₆ alkyl. In yet another embodiment R₉ is an optionally substituted alkyl group selected from: methyl, ethyl, n-propyl, isopropyl, n-butyl, tert-butyl, sec-butyl, iso-butyl, n-pentyl, 3-pentanyl, amyl, neopentyl, 3-methyl-2-butanyl, tertiary amyl, and n-hexyl, n-heptyl, and n-octyl.

In one embodiment R₉ is an optionally substituted alkenyl group. In another embodiment R₉ is an optionally substituted: C₂₋₁₀ alkenyl, C₂₋₉ alkenyl, C₂₋₈ alkenyl, C₂-7 alkenyl, C₂₋₆ alkenyl, C₂₋₅ alkenyl, C₂₋₄ alkenyl, C₂₋₃ alkenyl, or C₂ alkenyl. In yet another embodiment R₉ is an optionally substituted alkenyl group selected from: ethenyl, 1-propenyl, 2-propenyl, 1-butenyl, 2-butenyl, butadienyl, pentenyl, pentadienyl, hexenyl, heptenyl, octenyl, and octatrienyl.

In one embodiment R₉ is an optionally substituted alkynyl group. In another embodiment R₉ is an optionally substituted: C₂₋₂₀ alkynyl, C₂-10 alkynyl, C₂₋₉ alkynyl, C₂₋₈ alkynyl, C₂₋₇ alkynyl, C₂₋₆ alkynyl, C₂₋₅ alkynyl, C₂₋₄ alkynyl, C₂₋₃ alkynyl, or C₂ alkynyl. In yet another embodiment R₉ is an optionally substituted alkynyl group selected from: ethynyl, 1-propynyl, 2-propynyl, 1-butynyl, 2-butynyl, pentynyl, hexynyl, heptynyl, and octynyl.

In one embodiment R₉ is an optionally substituted carbocyclyl group. In another embodiment R₉ is an optionally substituted: C₃₋₁₀ carbocyclyl, C₃₋₈ carbocyclyl, C₃₋₆ carbocyclyl, or C₅₋₁₀ carbocyclyl. In yet another embodiment at least one of R₁ and R₂ is an optionally substituted carbocyclyl group selected from: cyclopropyl, cyclopropenyl, cyclobutyl, cyclobutenyl, cyclopentyl, cyclopentenyl, cyclohexyl, cyclohexenyl, cyclohexadienyl, cycloheptyl, cycloheptenyl, cycloheptadienyl, cycloheptatrienyl, cyclooctyl, cyclooctenyl, bicyclo[2.2.1]heptanyl, bicyclo[2.2.2]octanyl, cyclononyl, cyclononenyl, cyclodecyl, cyclodecenyl, octahydro-1H-indenyl, decahydronaphthalenyl, and spiro[4.5]decanyl.

In one embodiment R₉ is an optionally substituted O-alkyl group. In another embodiment R₉ is an O-alkyl group comprising an optionally substituted: C₁₋₂₀ alkyl, C₁₋₁₂ alkyl, C₁₋₁₀ alky, C₁₋₉ alkyl, C₁₋₈ alkyl, C₁₋₇ alkyl, C₁₋₆ alkyl, C₁₋₅ alkyl, C₁₋₄ alkyl, C₁₋₃ alkyl, C₁₋₂ alkyl, C₁ alkyl, or C₂₋₆ alkyl. In yet another embodiment R₉ is an O-alkyl group grouping comprising an optionally substituted alkyl group selected from: methyl, ethyl, n-propyl, isopropyl, n-butyl, tert-butyl, sec-butyl, iso-butyl, n-pentyl, 3-pentanyl, amyl, neopentyl, 3-methyl-2-butanyl, tertiary amyl, and n-hexyl, n-heptyl, and n-octyl.

In one embodiment a compound of Formula 1 is not oxaloacetic acid.

In another embodiment a compound of Formula 1 is not acetylacetone.

In yet another embodiment a compound of Formula 1 is not oxaloacetic acid nor acetylacetone.

In one embodiment at least one compound of Formula 1 is:

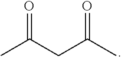

In one embodiment at least one compound of Formula 1 is:

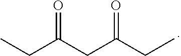

In one embodiment at least one compound of Formula 1 is:

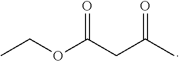

In one embodiment at least one compound of Formula 1 is:

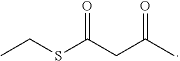

In one embodiment at least one compound of Formula 1 is:

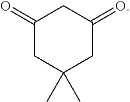

In one embodiment at least one compound of Formula 1 is:

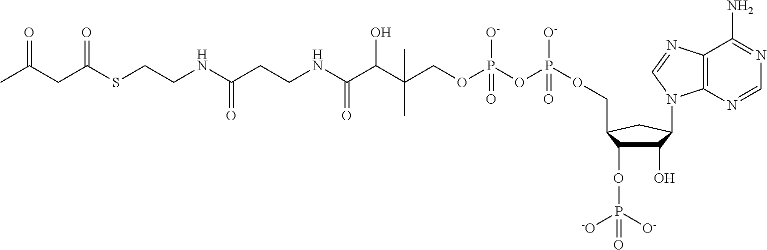

Herein a compound of Formula 1 may have a pKa (at 25° C., with water as the solvent reference) of about, or less than about: 11, 10.5, 10, 9.5, 9, 8.5, 8, 7.5, 7, 6.5, 6, 5.5, 5, 4.5, or 4.

### Polynucleotides and Genes

The present invention refers to various polynucleotides. As used herein, a “polynucleotide” or “nucleic acid” or “nucleic acid molecule” means a polymer of nucleotides, which may be DNA or RNA or a combination thereof, and includes genomic DNA, mRNA, cRNA, and cDNA. It may be DNA or RNA of cellular, genomic or synthetic origin, for example made on an automated synthesizer, and may be combined with carbohydrate, lipids, protein or other materials, labelled with fluorescent or other groups, or attached to a solid support to perform a particular activity defined herein, or comprise one or more modified nucleotides not found in nature, well known to those skilled in the art. The polymer may be single-stranded, essentially double-stranded or partly double-stranded. Basepairing as used herein refers to standard basepairing between nucleotides, including G:U basepairs. Preferred polynucleotides of the invention encode a vanadate-dependent haloperoxidase polypeptide as defined herein.

The present invention involves modification of gene activity and the construction and use of chimeric genes. As used herein, the term “gene” includes any deoxyribonucleotide sequence which includes a protein coding region or which is transcribed in a cell but not translated, as well as associated non-coding and regulatory regions. Such associated regions are typically located adjacent to the coding region or the transcribed region on both the 5′ and 3′ ends for a distance of about 2 kb on either side. In this regard, the gene may include control signals such as promoters, enhancers, termination and/or polyadenylation signals that are naturally associated with a given gene, or heterologous control signals in which case the gene is referred to as a “chimeric gene”. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene.

The term “gene” includes a synthetic or fusion molecule encoding all or part of the proteins of the invention described herein and a complementary nucleotide sequence to any one of the above. A gene may be introduced into an appropriate vector for extrachromosomal maintenance in a cell or, preferably, for integration into the host genome.

As used herein, a “chimeric gene” refers to any gene that comprises covalently joined sequences that are not found joined in nature. Typically, a chimeric gene comprises regulatory and transcribed or protein coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. In an embodiment, the protein coding region of a vanadate-dependent haloperoxidase gene is operably linked to a promoter or polyadenylation/terminator region which is heterologous to the vanadate-dependent haloperoxidase gene, thereby forming a chimeric gene.

The term “endogenous” is used herein to refer to a substance that is normally present or produced in an unmodified organism or cell. An “endogenous gene” refers to a native gene in its natural location in the genome of an organism. As used herein, “recombinant nucleic acid molecule”, “recombinant polynucleotide” or variations thereof refer to a nucleic acid molecule which has been constructed or modified by recombinant DNA/RNA technology. The terms “foreign polynucleotide” or “exogenous polynucleotide” or “heterologous polynucleotide” and the like refer to any nucleic acid which is introduced into the genome of a cell by experimental manipulations.

Foreign or exogenous genes may be genes that are inserted into a non-native organism or cell, native genes introduced into a new location within the native host, or chimeric genes. Alternatively, foreign or exogenous genes may be the result of editing the genome of the organism or cell, or progeny derived therefrom. A “transgene” is a gene that has been introduced into the genome by a transformation procedure. The term “genetically modified” includes introducing an exogenous polynucleotide such as a gene into cells by transformation or transduction, gene editing, mutating genes in cells and altering or modulating the regulation of a gene in a cell or organisms to which these acts have been done or their progeny.

Furthermore, the term “exogenous” in the context of a polynucleotide (nucleic acid) refers to the polynucleotide when present in a cell that does not naturally comprise the polynucleotide. The cell may be a cell which comprises a non-endogenous polynucleotide resulting in an altered amount of production of the encoded polypeptide, for example an exogenous polynucleotide which increases the expression of an endogenous polypeptide, or a cell which in its native state does not produce the polypeptide. Increased production of a polypeptide of the invention is also referred to herein as “over-expression”. An exogenous polynucleotide of the invention includes polynucleotides which have not been separated from other components of the transgenic (recombinant) cell, or cell-free expression system, in which it is present, and polynucleotides produced in such cells or cell-free systems which are subsequently purified away from at least some other components. The exogenous polynucleotide (nucleic acid) can be a contiguous stretch of nucleotides existing in nature, or comprise two or more contiguous stretches of nucleotides from different sources (naturally occurring and/or synthetic) joined to form a single polynucleotide. Typically, such chimeric polynucleotides comprise at least an open reading frame encoding a polypeptide of the invention operably linked to a promoter suitable of driving transcription of the open reading frame in a cell of interest.

The % identity of a polynucleotide is determined by GAP (Needleman and Wunsch, 1970) analysis (GCG program) with a gap creation penalty=5, and a gap extension penalty=0.3. The query sequence is at least 1,500 nucleotides in length, and the GAP analysis aligns the two sequences over a region of at least 1,500 nucleotides. Preferably, the query sequence is at least 1,800 nucleotides in length, and the GAP analysis aligns the two sequences over a region of at least 1,800 nucleotides. Even more preferably, the GAP analysis aligns two sequences over their entire length.

With regard to the defined polynucleotides, it will be appreciated that % identity figures higher than those provided above will encompass preferred embodiments. Thus, where applicable, in light of the minimum % identity figures, it is preferred that the polynucleotide comprises a polynucleotide sequence which is at least 60%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

In a further embodiment, the present invention relates to polynucleotides which are substantially identical to those specifically described herein. As used herein, with reference to a polynucleotide the term “substantially identical” means the substitution of one or a few (for example 2, 3, or 4) nucleotides whilst maintaining at least one activity of the native protein encoded by the polynucleotide. In addition, this term includes the addition or deletion of nucleotides which results in the increase or decrease in size of the encoded native protein by one or a few (for example 2, 3, or 4) amino acids whilst maintaining at least one activity of the native protein encoded by the polynucleotide.

### Nucleic Acid Constructs

Transgenic organisms and cells useful for the invention can be produced using nucleic acid contracts which encode the vanadate-dependent haloperoxidase.

The present invention refers to elements which are operably connected or linked. “Operably connected” or “operably linked” and the like refer to a linkage of polynucleotide elements in a functional relationship. Typically, operably connected nucleic acid sequences are contiguously linked and, where necessary to join two protein coding regions, contiguous and in reading frame. A coding sequence is “operably connected to” another coding sequence when RNA polymerase will transcribe the two coding sequences into a single RNA, which if translated is then translated into a single polypeptide having amino acids derived from both coding sequences. The coding sequences need not be contiguous to one another so long as the expressed sequences are ultimately processed to produce the desired protein.

As used herein, the term “cis-acting sequence”, “cis-acting element” or “cis-regulatory region” or “regulatory region” or similar term shall be taken to mean any sequence of nucleotides, which when positioned appropriately and connected relative to an expressible genetic sequence, is capable of regulating, at least in part, the expression of the genetic sequence. Those skilled in the art will be aware that a cis-regulatory region may be capable of activating, silencing, enhancing, repressing or otherwise altering the level of expression and/or cell-type-specificity and/or developmental specificity of a gene sequence at the transcriptional or post-transcriptional level. In preferred embodiments of the present invention, the cis-acting sequence is an activator sequence that enhances or stimulates the expression of an expressible genetic sequence.

“Operably connecting” a promoter or enhancer element to a transcribable polynucleotide means placing the transcribable polynucleotide (e.g., protein-encoding polynucleotide or other transcript) under the regulatory control of a promoter, which then controls the transcription of that polynucleotide. In the construction of heterologous promoter/structural gene combinations, it is generally preferred to position a promoter or variant thereof at a distance from the transcription start site of the transcribable polynucleotide which is approximately the same as the distance between that promoter and the protein coding region it controls in its natural setting; i.e., the gene from which the promoter is derived. As is known in the art, some variation in this distance can be accommodated without loss of function. Similarly, the preferred positioning of a regulatory sequence element (e.g., an operator, enhancer etc) with respect to a transcribable polynucleotide to be placed under its control is defined by the positioning of the element in its natural setting; i.e., the genes from which it is derived.

“Promoter” or “promoter sequence” as used herein refers to a region of a gene, generally upstream (5′) of the RNA encoding region, which controls the initiation and level of transcription in the cell of interest. A “promoter” includes the transcriptional regulatory sequences of a classical genomic gene, such as a TATA box and CCAAT box sequences, as well as additional regulatory elements (i.e., upstream activating sequences, enhancers and silencers) that alter gene expression in response to developmental and/or environmental stimuli, or in a tissue-specific or cell-type-specific manner. A promoter is usually, but not necessarily (for example, some PolIII promoters), positioned upstream of a structural gene, the expression of which it regulates. Furthermore, the regulatory elements comprising a promoter are usually positioned within 2 kb of the start site of transcription of the gene. Promoters may contain additional specific regulatory elements, located more distal to the start site to further enhance expression in a cell, and/or to alter the timing or inducibility of expression of a structural gene to which it is operably connected.

In an embodiment, the promoter is a constitutive promoter. An example of a constitutive promoter is the GAP (glyceraldehyde-3-phosphate dehydrogenase)-promoter. Other examples of promoters suitable for use in the invention include pTef1 (Translation elongation factor 1), pTDH3 (glyceraldehyde 3-phosphate dehydrogenase), pPGK1 (phosphoglycerate kinase), pYEF3 (Translation elongation factor 3), pRPL3 (Ribosomal Protein L3), pCCW12 (covalently linked cell wall protein), pENO2 (elonase).

In an embodiment, the promoter is an inducible promoter. Suitable inducible promoters include methanol inducible promoters. Methanol inducible promoters that can be used for yeast production include the promoters from AOX1 (aldehyde oxidase 1), AOX2 (aldehyde oxidase 2), CTA1 (peroxisomal catalase), DAS1 (dihydroxyacetone synthase 1), DAS2 (dihydroxyacetone synthase 2), FLD (formaldehyde dehydrogenase), and PMP20 (peroxisome membrane protein which has glutathione peroxidase activity). Other examples of inducible promoters suitable for use in the invention include pCUP1 (copper regulated), pFOX1 (Fatty Acid Oxidation 1), pFOX2 (Fatty Acid Oxidation 2), pCTA1 (oleate-inducible catalase A), pFAA2 (ADR1 transcription factor) and GSH1 (gamma glutamylcysteine synthetase 1).

In an embodiment, the promoter may be a galactokinase promoter, for example pGAL1, 2, 7 or 10. In an embodiment, promoters such as these may require engineering to switch off glucose repression of beta-oxidation. In an embodiment, the methods of the invention may utilise synthetic promoters, including those described in Tang et al. (2020).

In an embodiment, the promoter is a developmentally regulated promoter which is capable of driving expression of the introduced polynucleotide at an appropriate developmental stage.

Other cis-acting sequences which may be employed include transcriptional and/or translational enhancers. Enhancer regions are well known to persons skilled in the art, and can include an ATG translational initiation codon and adjacent sequences. When included, the initiation codon should be in phase with the reading frame of the coding sequence relating to the foreign or exogenous polynucleotide to ensure translation of the entire sequence if it is to be translated. Translational initiation regions may be provided from the source of the transcriptional initiation region, or from a foreign or exogenous polynucleotide. The sequence can also be derived from the source of the promoter selected to drive transcription, and can be specifically modified so as to increase translation of the mRNA.

The nucleic acid construct may comprise a 3′ non-translated sequence from, for example, about 50 to 1,000 nucleotide base pairs which may include a transcription termination sequence. A 3′ non-translated sequence may contain a transcription termination signal which may or may not include a polyadenylation signal and any other regulatory signals capable of effecting mRNA processing. A polyadenylation signal functions for addition of polyadenylic acid tracts to the 3′ end of a mRNA precursor. Polyadenylation signals are commonly recognized by the presence of homology to the canonical form 5′ AATAAA-3′ although variations are not uncommon. Transcription termination sequences which do not include a polyadenylation signal include terminators for Poll or PolIII RNA polymerase which comprise a run of four or more thymidines. Examples of suitable 3′ non-translated sequences are the 3′ transcribed non-translated regions containing a polyadenylation signal from an octopine synthase (ocs) gene or nopaline synthase (nos) gene of Agrobacterium tumefaciens (Bevan et al., 1983). Suitable 3′ non-translated sequences may also be derived from plant genes such as the ribulose-1,5-bisphosphate carboxylase (ssRUBISCO) gene, although other 3′ elements known to those of skill in the art can also be employed.

### Vectors

The present invention includes use of vectors for manipulation or transfer of nucleic acid constructs. A vector preferably is double-stranded DNA and contains one or more unique restriction sites and may be capable of autonomous replication in a defined host cell including a target cell or tissue or a progenitor cell or tissue thereof, or capable of integration into the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into a cell, is integrated into the genome of the recipient cell and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the cell into which the vector is to be introduced. The vector may also include a selection marker such as an antibiotic resistance gene, a herbicide resistance gene, a nutrient based marker or other gene that can be used for selection of suitable transformants. Examples of such genes are well known to those of skill in the art.

Preferably, the nucleic acid construct is stably incorporated into the genome of the organism or cells. Accordingly, the nucleic acid comprises appropriate elements which allow the molecule to be incorporated into the genome, or the construct is placed in an appropriate vector which can be incorporated into a chromosome of a plant cell.

The vector can be either RNA or DNA, either prokaryotic or eukaryotic, and typically is a virus or a plasmid.

### Recombinant Organisms and Cells

Transformation of a nucleic acid molecule into a cell can be accomplished by any method by which a nucleic acid molecule can be inserted into the cell. Transformation techniques include, but are not limited to, transfection, particle bombardment/biolistics, electroporation, microinjection, lipofection, adsorption, and protoplast fusion. In an embodiment, gene editing is used to modify the target cell genome using, for example, targeting nucleases such as TALEN, Cpf1 (Cas12a), MAD7 or Cas9-CRISPR or engineered nucleases derived therefrom.

Depending on the type of cell, a recombinant cell may remain unicellular or may grow into a tissue, organ or a multicellular organism. Transformed nucleic acid molecules of the present invention can remain extrachromosomal or can integrate into one or more sites within a chromosome of the transformed (i.e., recombinant) cell in such a manner that their ability to be expressed is retained.

Preferred host cells and organisms are discussed herein.

In an embodiment of the invention, the methods of the invention utilise organisms or cells with targeted modification or inhibition of catalase to promote the accumulation of H₂O₂ and in turn promotes VHPO activity. Catalase is found in nearly all living organisms exposed to oxygen and catalyzes the decomposition of H₂O₂ to water and oxygen.

In one example, the nucleotides encoding both copies of catalase in the genome of the organism or part thereof, microorganism or cells are modified. In another example, the nucleotides encoding the catalase are mutated, edited or deleted. Suitable methods for deleting or mutating endogenous genes (e.g., using site-specific or RNA-guided nucleases) are known in the art.

For instance, genome editing may be used which uses engineered nucleases composed of sequence specific DNA binding domains fused to a non-specific DNA cleavage module. These chimeric nucleases enable efficient and precise genetic modifications (including deletions, mutations and insertions) by inducing targeted DNA double stranded breaks that stimulate the cell's endogenous cellular DNA repair mechanisms to repair the induced break. Such mechanisms include, for example, error prone non-homologous end joining (NHEJ) and homology directed repair (HDR).

In the presence of donor plasmid with extended homology arms, HDR can lead to the introduction of single or multiple transgenes to correct or replace existing genes. In the absence of donor plasmid, NHEJ-mediated repair yields small insertion or deletion mutations of the target that cause gene disruption.

Engineered nucleases useful in the methods of the present invention include zinc finger nucleases (ZFNs) and transcription activator-like (TAL) effector nucleases (TALEN).

Typically nuclease encoded genes are delivered into cells by plasmid DNA, viral vectors or in vitro transcribed mRNA. The use of fluorescent surrogate reporter vectors also allows for enrichment of ZFN- and TALEN-modified cells. As an alternative to ZFN gene-delivery systems, cells can be contacted with purified ZFN proteins which are capable of crossing cell membranes and inducing endogenous gene disruption.

A zinc finger nuclease (ZFN) comprises a DNA-binding domain and a DNA-cleavage domain, wherein the DNA binding domain is comprised of at least one zinc finger and is operatively linked to a DNA-cleavage domain. The zinc finger DNA-binding domain is at the N-terminus of the protein and the DNA-cleavage domain is located at the C-terminus of said protein.

A ZFN must have at least one zinc finger. In a preferred embodiment, a ZFN would have at least three zinc fingers in order to have sufficient specificity to be useful for targeted genetic recombination in a host cell. Typically, a ZFN having more than three zinc fingers would have progressively greater specificity with each additional zinc finger.

The zinc finger domain can be derived from any class or type of zinc finger. In a particular embodiment, the zinc finger domain comprises the Cis₂His₂ type of zinc finger that is very generally represented, for example, by the zinc finger transcription factors TFIIIA or Sp1. In a preferred embodiment, the zinc finger domain comprises three Cis₂His₂ type zinc fingers. The DNA recognition and/or the binding specificity of a ZFN can be altered in order to accomplish targeted genetic recombination at any chosen site in cellular DNA. Such modification can be accomplished using known molecular biology and/or chemical synthesis techniques.

The ZFN DNA-cleavage domain is derived from a class of non-specific DNA cleavage domains, for example the DNA-cleavage domain of a Type II restriction enzyme such as FokI (Kim et al., 1996). Other useful endonucleases may include, for example, HhaI, HindIII, Nod, BbvCI, EcoRI, BglI, and AlwI.

In order to target genetic recombination or mutation according to a preferred embodiment of the present invention, two 9 bp zinc finger DNA recognition sequences must be identified in the host microbial cell DNA. These recognition sites will be in an inverted orientation with respect to one another and separated by about 6 bp of DNA. ZFNs are then generated by designing and producing zinc finger combinations that bind DNA specifically at the target locus, and then linking the zinc fingers to a DNA cleavage domain.

ZFN activity can be improved through the use of transient hypothermic culture conditions to increase nuclease expression levels (Doyon et al., 2010) and co-delivery of site-specific nucleases with DNA end-processing enzymes (Certo et al., 2012). The specificity of ZFN-mediated genome editing can be improved by use of zinc finger nickases (ZFNickases) which stimulate HDR without activation the error-prone NHE-J repair pathway (Kim et al., 2012)

A transcription activator-like (TAL) effector nuclease (TALEN) comprises a TAL effector DNA binding domain and an endonuclease domain.

TAL effectors are proteins of plant pathogenic bacteria that are injected by the pathogen into the plant cell, where they travel to the nucleus and function as transcription factors to turn on specific plant genes. The primary amino acid sequence of a TAL effector dictates the nucleotide sequence to which it binds. Thus, target sites can be predicted for TAL effectors, and TAL effectors can be engineered and generated for the purpose of binding to particular nucleotide sequences.

Fused to the TAL effector-encoding nucleic acid sequences are sequences encoding a nuclease or a portion of a nuclease, typically a nonspecific cleavage domain from a type II restriction endonuclease such as FokI (Kim et al., 1996). Other useful endonucleases may include, for example, HhaI, HindIII, Nod, BbvCI, EcoRI, BglI, and AlwI. The fact that some endonucleases (e.g., FokI) only function as dimers can be capitalized upon to enhance the target specificity of the TAL effector. For example, in some cases each FokI monomer can be fused to a TAL effector sequence that recognizes a different DNA target sequence, and only when the two recognition sites are in close proximity do the inactive monomers come together to create a functional enzyme. By requiring DNA binding to activate the nuclease, a highly site-specific restriction enzyme can be created.

A sequence-specific TALEN can recognize a particular sequence within a preselected target nucleotide sequence present in a cell. Thus, in some embodiments, a target nucleotide sequence can be scanned for nuclease recognition sites, and a particular nuclease can be selected based on the target sequence. In other cases, a TALEN can be engineered to target a particular cellular sequence.

Distinct from the site-specific nucleases described above, the clustered regulatory interspaced short palindromic repeats (CRISPR)/Cas system provides an alternative to ZFNs and TALENs for inducing targeted genetic alterations. In bacteria, the CRISPR system provides acquired immunity against invading foreign DNA via RNA-guided DNA cleavage.

CRISPR systems rely on CRISPR RNA (crRNA) and transactivating chimeric RNA (tracrRNA) for sequence-specific silencing of invading foreign DNA. Three types of CRISPR/Cas systems exist: in type II systems, Cas9 serves as an RNA-guided DNA endonuclease that cleaves DNA upon crRNA-tracrRNA target recognition. CRISPR RNA base pairs with tracrRNA to form a two-RNA structure that guides the Cas9 endonuclease to complementary DNA sites for cleavage.

CRISPR loci are a distinct class of interspersed short sequence repeats (SSRs) that were first recognized in E. coli (Ishino et al., 1987; Nakata et al., 1989). Similar interspersed SSRs have, been identified in Haloferax mediterranei, Streptococcus pyogenes, Anabaena, and Mycobacterium tuberculosis (Groenen et al., 1993).

The CRISPR loci differ from other SSRs by the structure of the repeats, which have been termed short regularly spaced repeats (SRSRs) (Janssen et al., 2002; Mojica et al., 2000). The repeats are short elements that occur in clusters that are always regularly spaced by unique intervening sequences with a constant length (Mojica et al., 2000). Although the repeat sequences are highly conserved between strains, the number of interspersed repeats and the sequences of the spacer regions differ from strain to strain (van Embden et al., 2000).

The common structural characteristics of CRISPR loci are described in Jansen et al. (2002) as (i) the presence of multiple short direct repeats, which show no or very little sequence variation within a given locus; (ii) the presence of non-repetitive spacer sequences between the repeats of similar size; (iii) the presence of a common leader sequence of a few hundred basepairs in most species harbouring multiple CRISPR loci; (iv) the absence of long open reading frames within the locus; and (v) the presence of one or more cas genes.

CRISPRs are typically short partially palindromic sequences of 24-40 bp containing inner and terminal inverted repeats of up to 11 bp. Although isolated elements have been detected, they are generally arranged in clusters (up to about 20 or more per genome) of repeated units spaced by unique intervening 20-58 bp sequences. CRISPRs are generally homogenous within a given genome with most of them being identical. However, there are examples of heterogeneity in, for example, the Archaea (Mojica et al., 2000).

As used herein, the term “cas gene” refers to one or more cas genes that are generally coupled associated or close to or in the vicinity of flanking CRISPR loci. A comprehensive review of the Cas protein family is presented in Haft et al. (2005). The number of cas genes at a given CRISPR locus can vary between species.

In an example, the nucleotides encoding both copies of catalase in the genome of S. cerevisiae (CTA1: YDR256C and CTT1: YGR088W) are modified or deleted. In another example, the nucleotides encoding both copies of the three catalase genes in the genome of Yarrowia lipolytica (CTA1: YDR256C and CTT1: YGR088W) are modified or deleted. In another example, the nucleotides encoding both copies of catalase in the genome of E. coli (KatE (GenBank: AAT48137.1) and KatG (GenBank: AAC76924.1)) are modified or deleted. In yet another example, the nucleotides encoding both copies of catalase in the genome of Bacillus subtilis (KatA (GenBank: CAB04807.1), KatE (GeneBank: CAB15931.2) and KatX (GeneBank: CAB15889.1) are modified or deleted.

It will be understood that a FabG or FAS or catalase gene(s) modification may include a knock-out of one or more FabG or FAS or catalase genes or may include a knock down of one or more FabG or FAS or catalase genes. As understood in the art, gene knockout is the complete elimination of genes from an organism. Gene knockdown is the reduction of the expression of a gene in an organism and may refer to knockdown by 30%, 40%, 50%, 60%, 70%, 80%, 90% or more. Evaluation of gene knockdown may be confirmed using routine means in the art including measurement of gene levels by reverse transcription or real-time PCR. In another embodiment the transcription or the activity of the catalase protein may be inhibited using RNAi or a compound to promote the accumulation of H₂O₂. Suitable means for inhibiting catalase activity include the use of 3-amino-1,2,4-triazol.

In an embodiment, the targeting of genes that regulate fatty acid β-oxidation may be used in the methods of the invention. In an example, FabG and similar genes may be targeted, for example, E. coli FabG (GenBank: AAC74177.1). In an example, the 3-ketoacyl-CoA reductase and similar genes may be targeted, for example, S. cerevisiae (GenBank: AY557868, Uniprot P38286).

In another embodiment the transcription or the activity of the protein encoded by the genes that regulate the 3-ketoacyl-CoA reductase may be inhibited using RNAi or a compound to promote the accumulation of acetoacyl-acyl-carrier-proteins. Suitable means for inhibiting 3-ketoacyl-CoA reductases such as FabG include the use of tannic acid.

In another embodiment the targeting of genes that regulate fatty acid synthase in the cellular FAS complex may be effective in promoting the accumulation of acetoacyl-acyl-carrier-proteins. FAS complexes in yeast and fungi are multifunctional protein complexes, composed of alpha and beta subunits that integrate and drive de novo fatty acid synthesis. Examples of suitable eukaryotic FAS genes include GenBank: AAA34601.1 (S. cerevisae) and GenBank: CAG83349.1 (Y. lipolytica).

Preferably, tannic acid is used alongside 3-amino-1,2,4-triazol.

As used herein, “a compound that promotes the accumulation of acetoacyl-acyl-carrier-proteins” is to be understood to mean any compound that inhibits or decreases the activity of the 3-ketoacyl-CoA reductase or FabG or equivalent enzyme such that the acetoacyl-acyl-carrier-protein can accumulate within the cell.

Yet additional means for enhancing metabolite availability for bromoform production include inducing acetoacyl CoA production from 2× acetyl CoA with acetoacetyl-CoA thiolase in E. Coli; targeting ERG10 (GenBank: AAA62378.1) in S. cerevisae and (GenBank: CAG82888.1) in Y. lipolytica; or targeting acetyl CoA+malonyl CoA with acetyl-CoA:malonyl-CoA acyltransferase (GenBank: BAJ10048.1) in Streptomyces sp. CL190.

In another embodiment, other inhibitors of catalase activity that may be utilised in the methods of the invention to promote the accumulation of H₂O₂ include 2,2′-azino-bis(3-ethylbenzthiazoline-6-sulfonic acid), 3-amino-1,2,4-triazole, 3-amino-4-hydroxybenzoic acid, azide, Ba²⁺, Co²⁺, Cu²⁺, EDTA, H₂O₂, KCl, MgCl₂, NaCl and nicotinic acid hydrazide.

As used herein, the term catalase activity inhibitor is understood to mean any inhibitor that reduces catalase activity such that it is effective in reducing the intracellular conversion of H₂O₂ to O₂ gas. In an embodiment, the catalase activity is reduced by at least 50%, at least 60%, at least 70%, at least 80%, at least 90%, at least 99%, by 100%, between 50% and 100%, between 70% and 100% or between 80% and 100%.

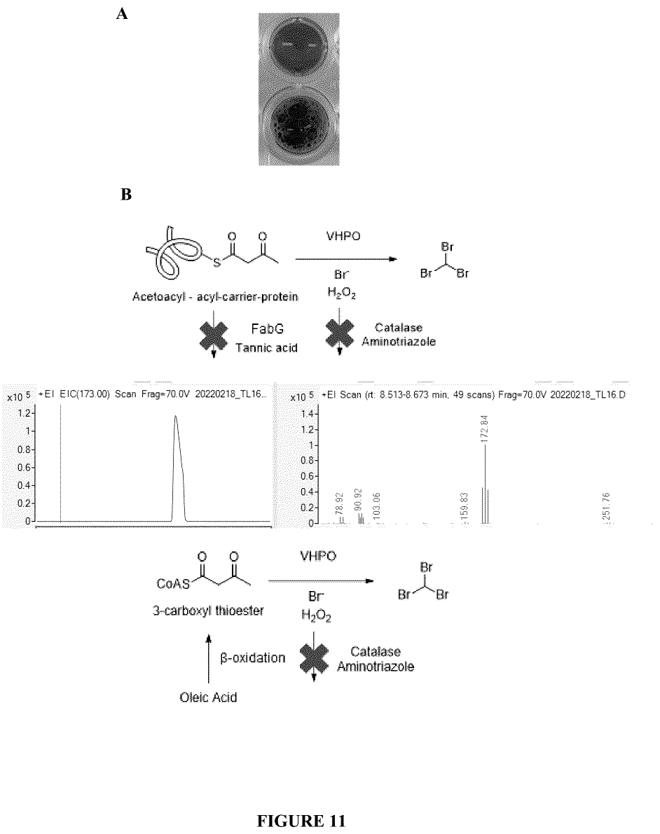

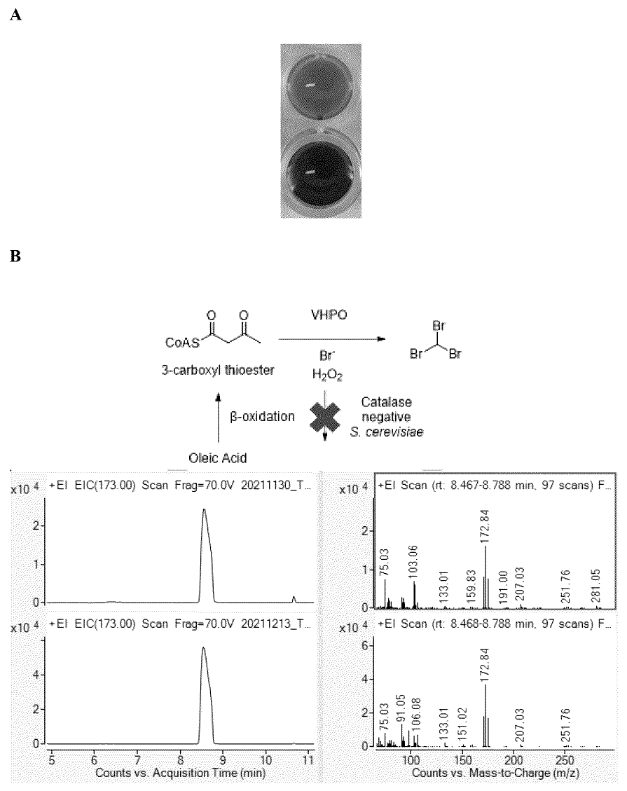

Additional approaches may be used to promote the production of bromoform in the methods of the invention including targeting the 3-carboxyl thioester intermediate of β-oxidation (FIG. 11). In one example, this may be achieved by providing a fatty acid in the media such as oleic acid to induce β-oxidation, preferably in the presence of 3-amino-1,2,4-triazol. In another example, a medium or long chain fatty acid may be used as a carbon source. As used herein, any suitable conditions that promote β-oxidation may be used in the methods of the invention. The term “promotes β-oxidation” is intended to mean a compound capable of increasing β-oxidation in an organism or part thereof, microorganism or cells thereof.

In an embodiment, the plants are fodder plants. Examples of fodder plants include, but are not limited to, legumes such as alfalfa, lucerne and soybean, cereals such as barley, sorghum, corn, millet, oats, and wheat, common duckweed, Brassica spp., kale, turnip, clover such as red clover, subterranean clover and white clover, and grasses such as bermuda grass, fescue and ryegrass.

In an embodiment, the Cyanobacteria is selected from Spirulina (Arthrospira) sp. such as Arthrospira platensis, Anabaena sp., Nostoc sp. such as Nostoc commune, Aphanizomenon sp. such as Aphanizomenon flos-aquae, Chlorella sp., Scendesmus sp. and Synechococcus sp. (CCMP 2515).

In an embodiment, the organism is a macroalage (seaweed). Examples include, but are not limited to, Asparagopsis taxiformis, Asparagopsis armata, Ulva lactuca, Chaetomorpha linum, Ascophyllum nodosum (brown alga) and Laminaria digitate.

In an embodiment, the algae is a diatom such as Phaeodactylum tricornutum (CCMP 633 and 632).

In an embodiment, the Alternaria sp. is A. alternata, A. arborescens, A. burnsii or A. didymospora.

In an embodiment, the Drechslera sp. is D. halodes.

In an embodiment, the Curvularia sp. is C. cymbopogonis, C. inaequalis, C. lunata, C. verruculosa, C. specifera, C. pellescens or C. clavate.

In an embodiment, the Botrytis sp. is B. cinerea.

In an embodiment, the Bipolaris sp. is B. sorokiniana, B. victoriae, B. oryzae, B. zeicola or B. maydis.

In an embodiment, the Ulocladium sp. is U. chartarum.

### Production of Bromoform

Conditions for incubating, such as culturing, organisms (such as microorganisms), or part thereof, cells, a lysate of the organism or cells, or a mixture thereof, are well known to those in the art.

The incubation/culturing may be conducted at any suitable temperature such that vanadate-dependent haloperoxidase activity is maintained. For example, between 15° C. and 30° C., between 20° C. and 27° C. or about 25° C.

The microorganisms or cells are cultivated in a nutrient medium suitable for production and activity of the polypeptide using methods known in the art. For example, the microorganisms or cells may be cultivated by shake flask cultivation, or small-scale or large-scale fermentation (including continuous, batch, fed-batch, or solid state fermentations) in laboratory or industrial fermenters in a suitable medium and under conditions allowing for the production of bromoform. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection).

Where the methods of the invention contemplate the use of low glucose media (such as low glucose SDUGGO media), it is envisaged that YPD based media may be used, especially where genes are incorporated into the genome so the selection pressure provided by dropout media is not required to maintain plasmid integrity. Other low cost/undefined media may also be useful e.g. soy meal, waste cooking oil, whey, potato dextrose media, minimal media, synthetic defined media without uracil, LB for E. coli, methanol and ethanol. Further, buffering of the media (e.g., with MOPS (pH6.5) or HEPES may also assist bromoform production. Methods such as these may be suitable for culturing a number of different organisms including Yarrowia lipotlytica and Saccharomyces cerevisiae. In another example, culturing of Curvularia comprises media composed of 0.5% yeast extract, 1-10 g/L Glucose, 10 μM ZnSO4, 9 μM K2HPO4, 8 μM MnCl2, 5.5 μM FeSO4, 5 μM CuSO4 and 0.4 g/L Agar.

In an embodiment, the incubating occurs in the presence of hydrogen peroxide. In an embodiment, the concentration of hydrogen peroxide is at least 20 mM, at least 50 mM, at least 80 mM, at least 90 mM, at least 100 mM, at least 500 mM, between 50 mM and 1 M, between 50 mM and 100 mM or between 500 mM and 1 M.

In an embodiment the H₂O₂ (100 mM) is added at least once, at least two times, at least 3 times one or at least four times at about 24 hour intervals, or daily, to increase bromoform production.

In an embodiment, the incubating occurs in the presence of bromide, such as sodium bromide or potassium bromide. In an embodiment, the concentration of bromide is at least 100 mM, at least 250 mM, at least 500 mM, between 100 mM and 1 M, between 200 mM and 750 mM or between 300 mM and 500 mM.

In an embodiment, the incubating occurs in the presence of vanadium, such as sodium orthovanadate. In an embodiment, the concentration of vanadium is at least 0.1 mM, at least 0.5 mM, at least 1 mM, between 0.1 mM and 10 mM, between 0.5 mM and 5 mM or between 0.75 mM and 1.25 mM.

In an embodiment, the method produces at least 1 g/L, at least 2 g/L, at least 5 g/L, or between 5 g/L and 10 g/L, of bromoform, for instance when cultured in accordance with the method or incubated in medium comprising the vanadate-dependent haloperoxidase.

In one example, whole culture (meaning media and biomass) can be supplemented with N-(2-Hydroxyethyl) piperazine-N′-(2-ethanesulfonic acid) (HEPES, 100 mM, pH 7.6), Na₃VO₄, (1 mM) dimidone (20 mM) KBr (100 mM) and H₂O₂ (100 mM) added at about 24 hour intervals until bromoform concentration reaches a maximum.

In one embodiment, the methods as provided may be carried out in a fermenter. The fermentation can be carried out in three different modes: batch, fed-batch and continuous mode. A standard batch bioreactor is considered a “closed” system. In batch mode, all the media components are added to bioreactor while ensuring the sterility. Once the medium has been prepared, the bioreactor is inoculated with an appropriate inoculum and the fermentation is allowed to proceed until the end without any changes to the medium, i.e., without feeding of any additional components. Components such as acid and/or base can, however, be added to maintain the pH, and air/oxygen can be added to maintain the dissolved oxygen levels. In batch fermentation biomass and product concentration change over time until the fermentation is complete. The cells undergo classical lag-phase, exponential growth-phase, stationary phase growth, followed by death phase.

In some embodiments, the methods may involve harvesting of the bromoform containing organisms (such as microorganisms) or cells from the culture. Harvesting can be by any method known in the art such as centrifugation, for example in a centrifugal pump (a separator), filtration, or settling.

In other embodiments, the methods may involve harvesting the supernatant or volatiles containing bromoform emitting from the supernatant or media.

### Food, Feedstuffs, Drinks and Supplements

Bromoform produced using the invention can be used to produce food, feedstuffs, drinks or supplements. For purposes of the present invention, “food” or “feedstuffs” include any food or preparation for human or animal consumption which when taken into the body to one or more or all of (a) serve to nourish or build up tissues or supply energy; (b) maintain, restore or support adequate nutritional status or metabolic function; and (c) suppress the growth of methanogenic bacteria, such as those in the rumen of a ruminant.

Food, feedstuffs, drinks or supplements of the invention may include a suitable carrier(s). The term “carrier” is used in its broadest sense to encompass any component which may or may not have nutritional value. As the skilled addressee will appreciate, the carrier must be suitable for use (or used in a sufficiently low concentration) in a food, feedstuff, drink or supplement such that it does not have deleterious effect on an organism which consumes the food, feedstuff, drink or supplement.

The food, feedstuff, drink or supplement may include edible macronutrients, protein, carbohydrate, vitamins, and/or minerals in amounts desired for a particular use. The amounts of these ingredients will vary depending on whether the composition is intended for use with normal individuals or for use with individuals having specialized needs.

Examples of suitable carriers with nutritional value include, but are not limited to, macronutrients such as edible fats, carbohydrates and proteins.

With respect to vitamins and minerals, the following may be added to the food, feedstuff, drink or supplement compositions of the present invention: calcium, phosphorus, potassium, sodium, chloride, magnesium, manganese, iron, copper, zinc, selenium, iodine, and Vitamins A, E, D, C, and the B complex. Other such vitamins and minerals may also be added.

An animal feed supplement is a concentrated additive or premix which is either added to animal feed (for example in a feedlot) or is available to an animal (for example a lick-block). In one embodiment, the animal feed supplement is the organism, particularly a microorganism, or an extract or lysate thereof comprising bromoform, in the form of a powder or compacted or granulated solid.

The components utilized in the food, feedstuff, drink or supplement compositions of the present invention can be of semi-purified or purified origin. By semi-purified or purified is meant a material which has been prepared by purification of a natural material or by de novo synthesis.

In an embodiment, the feedstuff or supplement is an animal feed, in particular a feed or supplement for a ruminant animal.

### EXAMPLES

### Example 1—General Methods

### Reagents

Chemical reagents were purchased from Merck (Germany), DNA polymerases and other reagents for molecular biology were purchased from New England Biolabs (Ma, USA). Plasmid DNA was purified using the Miniprep kit from Qiagen (Germany), and gel purifications were carried out with the PureLink Quick gel extraction kit from Thermo-Scientific (Ma, USA). Oleic acid used in cell culture was technical grade, 90% purity.

### Strains and Media

DH5α E. coli was used for plasmid construction, and BL21 (DE3) E. coli was used for protein expression. Both strains were grown in LB supplemented with 100 mg/L ampicillin, or 30 mg/L chloramphenicol as necessary and incubated at 37° C., 220 rpm unless otherwise stated. Cells were made competent by sequential treatment with MgCl₂ and CaCl₂) for transformation by heat shock (42° C., 30 seconds). The S. cerevisiae strain BY4741 (MATa his3Δ1 leu2Δ0 met15Δ0 ura3Δ0) (ATCC no. 201388) was used for all yeast experiments. For genetic manipulation cultures were grown on YPD media (1% Bacto yeast extract, 2% Bacto peptone, 2% glucose) or synthetic defined media (SD) without uracil (0.67% yeast nitrogenous base without amino acids, 0.192% Merck yeast synthetic dropout medium supplement without uracil, and 2% glucose).

### Plasmid Construction

The VHPO gene from the marine cyanobacterium Acaryochloris marina MBIC1101712 was synthesized by Epoch Life Science Inc. (Tx, USA) using the published gene sequence (GenBank Accession CP000828-nucleotides 5020736-5018817). This gene sequence was codon optimized for E. coli expression and cloned into expression vector pACYCDuet1 under the regulatory control of a LacI repressor and T7 promoter and terminator sequences. This same gene was also codon optimised for S. cerevisiae and synthesized by Epoch Life Science Inc. (Tx, USA) and cloned into expression vector pYES2 under the regulatory control of the Gal1 promoter. The DNA sequence encoding the ePST1 tag for localisation to the peroxisome (ttgggaagaggtaggcgctccaaactt) was later added to this gene using the Q5 site directed mutagenesis kit from New England biolabs (Ma, USA) according to the manufacturer's instructions. Primer sequences are given in Table 2. Subsequently, the sequence encoding VHPO-ePST1 was amplified by PCR and used with other PCR fragments from the pRS316 vector (Merck) and the pTEF2 promoter from BY4741 gDNA to construct the pRS-ePST1VHPO plasmid using the NEBuilder Hifi DNA assembly kit according to the manufacturer's instructions. The primers used were designed with the Gibson assembly wizard from Benchling (Ca, USA), and are given in Table 2 (all “t” nucleotides will be understood to be “u” in RNA form). BY4741 cells were transformed by the lithium acetate method described by Gietz et al. (1992).

TABLE 2
Primer sequences

CTT1 deletion
CTT1-999F
TGCCAAGTACATAGAATCCAC
(SEQ ID NO: 23)
CTT1-1R
AGACAAGAGAAGGATTTTTTTAATAAG
(SEQ ID NO: 24)
CTT1 Ura3F
cttattaaaaaaatccttctcttgtctTGTAC
AAGCACTAATATTTCATACTTATCGTACTTCA
TACtgattcggtaatctccga
(SEQ ID NO: 25)
CTT1 Ura3 R
TACGACCAATTTGTAATAACTAGTGATGTAGAG
AATAAACcttcctttttcaatgggtaataac
(SEQ ID NO: 26)
CTA1 deletion
CTA1-999F
GAACATCCAAATTCGGAACTAC
(SEQ ID NO: 27)
CTA1-3R
CTAGGGTTCCAAATTTATTTGTAAG
(SEQ ID NO: 28)
CTA1 Ura3F
cttacaaataaatttggaaccctagGTTAAAAA
AATTATCATTTCACACATAGGAAAGCTCGTCGt
gattcggtaatctccgaac
(SEQ ID NO: 29)
CTA1 Ura3R
TTTCTAACACGAGAATATGAGCATCACCTATTAG
TCGTATcttcctttttcaatgggtaataac
(SEQ ID NO: 30)
Site directed mutagenesis
Am VHPO-ePTS1R
ctacctcttcccaaGATACGGATGGTCGAACC
(SEQ ID NO: 31)
Am VHPO-ePTSIF
gcgctccaaacttTAGTCTAGAGGGCCGCAT
(SEQ ID NO: 32)
pRS-ePST1VHPO assembly
pRS316 FWD
GCTCGAAGGCTTTAATTTGCgaaccgtaaaaa
ggccgcgt
(SEQ ID NO: 33)
pRS316 REV
TATATGTAAGTATACGGCCCgccctgatagacgg
tttttcg
(SEQ ID NO: 34)
pTEF2 FWD
gaaaaaccgtctatcagggGGGCCGTATACTTAC
ATATA
(SEQ ID NO: 35)
pTEF2 REV
CTCCTAGTGTTCATAAGCTTCATGTTTAGTTAAT
TATAGTTCGT
(SEQ ID NO: 36)
pYES2-Am VBPO-SKL FWD
ACTATAATTAACTAAACATGAAG
CTTATGAACACTAGGAG
(SEQ ID NO: 37)
pYES2-Am VBPO-SKL REV
acgcggcctttttacggttcGCA
AATTAAAGCCTTCGAGC
(SEQ ID NO: 38)
pRS-ePST1VHPO sequencing
pRS seqF1
ggaacgaaaactcacgttaaggg
(SEQ ID NO: 39)
AmpR seqF
gagcaaaaacaggaaggcaaaatg
(SEQ ID NO: 40)
pRS seqF2
tgtcggggctggcttaac
(SEQ ID NO: 41)
Ura3 seqF
attatgacacccggtgtggg
(SEQ ID NO: 42)
Ptef2 SeqF
CGCTTCCCCTGCCGG
(SEQ ID NO: 43)
Am VHPO Seq intF
TGGTGATGGAGCCAAACTTAGG
(SEQ ID NO: 44)
tCYC1 seqF
TCACGCCCTCCCCCC
(SEQ ID NO: 45)
CYC1TerR
GGACCTAGACTTCAGGTTG
(SEQ ID NO: 46)

Gene Deletions in S. cerevisiae

The coding sequences of both catalase genes were deleted sequentially from the genome of S. cerevisiae BY4741 using the method detailed by Akada et al. (2006). Deletion of the coding regions was confirmed by PCR, as shown in FIG. 2. Primers used for this procedure are given in Table 2.

### Visualization of VHPO Activity

VHPO activity was simply and conveniently confirmed by bromination of phenol red to form bromophenol blue. This assay could be conducted on whole cells taken directly from cell culture experiments. For samples containing a high VHPO activity, for example recombinant E. coli, 100 μL of culture could be quickly centrifuged and the cells resuspended in 200 μL of assay mixture (100 mM HEPES (pH 7.4) 20 mM KBr, 1 mM Na₃VO₄, 100 mM H₂O₂) and typically the color change from red, through violet to blue was obvious within a few minutes. The relatively high H₂O₂ concentration was necessary to compensate for catalase activity present in the cells. For samples with a low activity, for example peroxisome targeted VHPO expressed in S. cerevisiae grown on glucose the color change would only be visible after 18 hours. For samples where no catalase was present the H₂O₂ concentration was lowered to 10 mM, as higher concentrations led to decolorization of the sample.

Expression of VHPO in E. coli

E. coli isolate BL21 (DE3) cells were transformed with the pACYC-AmVHPO construct. Transformants were selected on LB agar containing 30 mg/L chloramphenicol. Cultures were inoculated with 1 mL/L of overnight culture grown from a single colony until an OD600 of ˜0.5. IPTG 0.2 mM was then added, and the cultures transferred to 28° C. with shaking at 220 rpm for a further 18 hours.

Bromoform Synthesis from Commercial 1,3-dicarbonyls with Recombinant VHPO

E. coli cells from VHPO expression described above were resuspended in 1 mL water/100 mL culture volume and 20% v/v of this cell suspension was added to a solution of 100 mM HEPES (pH 7.4), 20 mM KBr, 1 mM Na₃VO₄, and 50 mM H₂O₂ along with 1 mM acetone, ethylacetoactate, acetyl acetone, 5,5-dimethyl-1,3-cyclohexanedione (dimedone). A sample with no additional substrate was also run as a control experiment. Samples (1 mL) were incubated in sealed tubes at room temperature for 24 hours. 10 μL samples from each were withdrawn and added to 1 mL brine to improve phase separation16 and placed in a 20 mL GC vial, which were then sealed with magnetic screw caps (Agilent). The head space of each sample was analyzed by solid phase microextraction (SPME) GC-MS.

Bromoform Biosynthesis in E. coli

VHPO was expressed in E. coli isolate BL21 (DE3) as described above. Alongside IPTG (0.2 mM) the culture was also supplemented with 50 mM KBr, 1 mM Na₃VO₄, 10 mM 3-amino,1,2,4-triazole, and 0.5 g/L tannic acid or 0.1% oleic acid (emulsified with 1% tergitol (Merck, Ma USA)). Typically, 10 mL LB cultures were grown in 50 mL tubes with the lids closed. Vented tubes led to the loss of bromoform by evaporation. Cells were harvested by centrifugation and resuspended in 1 mL of brine (6 M NaCl), transferred into 20 mL headspace GC vials and sealed with a magnetic screw cap before analysis by SPME-GC-MS.

Bromoform Synthesis by S. cerevisiae

10 mL seed cultures of were inoculated with a single colony of BY4741 transformed with pRS-ePST1-AmVHPO in SD and grown at 220 rpm, 30° C. overnight. These were used to inoculate 10 mL cultures of SDUGGO media (uracil (0.67% yeast nitrogenous base without amino acids, 0.192% Merck yeast synthetic dropout medium supplement without uracil, 10% glycerol, 0.1% glucose, 100 mM KBr, 1 mM Na₃VO₄, 100 mM MOPS (pH 6.5), 0.1% oleic acid emulsified in 1% tergitol) at an initial OD600 of 2.0, and incubated for a further 24 hours at 220 rpm, 30° C. As with E. coli, samples were grown in 50 mL tubes with the lids closed to minimize the loss of bromoform by evaporation. Cells were harvested by centrifugation and resuspended in 1 mL of brine (6 M NaCl), transferred into 20 mL headspace GC vials and sealed with a magnetic screw cap before analysis by SPME-GC-MS.

### Bromoform Detection by SPME-GC-MS

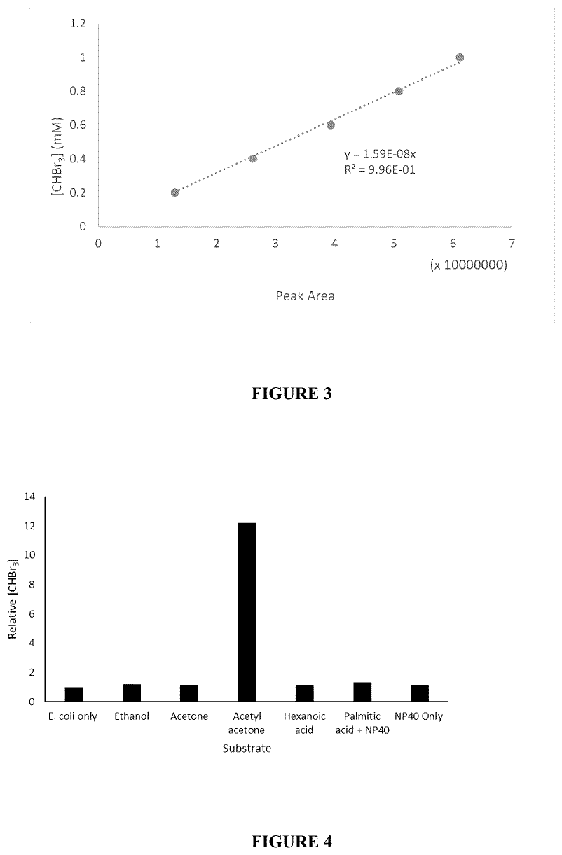

Depending on instrument availability, SPME-GC-MS was performed using either; a 7890A series gas chromatograph, a 5975C inert XL MSD mass selective detector and a MPS2 Gerstel multipurpose autosampler; or an 8890 series gas chromatograph, a 7250 Q/TOF mass selective detector and a MPS3 2XL Gerstel multipurpose autosampler. The headspace of each sample was sampled for 30 minutes at 30° C. by adsorption on a divynlbenzene/carboxen/polydimethylsiloxane fibre (Supleco, Pa, USA) before desorption in the sample inlet at 250° C. for 5 minutes in splitless injection mode with a 1.0 mm straight, no-wool liner. Chromatographic separation was carried out with a non-polar Agilent VF-5 ms column (30m×0.25 mm×0.25 μm) equipped with a 10 m EZ-Guard column. Helium (ultra-high purity, BOC, Australia) carrier gas was used at a flow rate of 1 mL/min. Aux transfer line was set to 320° C., ion source, 250° C. and quadrupole 150° C. Electron impact ionisation energy (EI) was 70 eV, with full MS scan from M/z 40-500 and a 3 minutes solvent delay. The oven temperature program began at 40° C. for 2 min, then ramped to 150° C. at 5° C./min, held for 2 minutes before ramping to 320° C. at a rate of 15° C./minutes and held for 1 minutes. GC/MS run time was 38 minutes. The fibre was re-conditioned for 10 minutes at 250° C., in the inlet between samples to prevent sample carryover. Bromoform was identified in samples by comparison of the retention time with an authentic standard (Merck, USA) and by mass spectral matching using the NIST/EPA/NIH Mass Spectral Library (version 2014) using the identify algorithm in Agilent's Mass Hunter Qualitative Analysis software (version 10.0). Bromoform was quantified by comparison of the signal corresponding to the molecular ion (m/z=249±0.5) using with a standard curve prepared from authentic samples of bromoform in brine in the same way as the samples to account for any matrix effects. The resulting calibration curve is shown in FIG. 3.

### Example 2—Screening of Transgenic Escherichia coli Expressing the Acaryochloris marina Haloperoxidase Gene for Increased Bromoform Production

The haloperoxidase gene from the marine cyanobacterium Acaryochloris marina MBIC11017 (Frank et al., 2016) (encoding SEQ ID NO:1) was synthesized by Epoch Life Science Inc. (Texas USA) using the published gene sequence (GenBank Accession CP000828-nucleotides 5020736-5018817). This gene sequence was codon optimized for E. coli expression (SEQ ID NO:17) and cloned into expression vector pACYCDuet1 under the regulatory control of a LacI repressor and T7 promoter and terminator sequences. E. coli isolate BL21 (DE3) cells were made competent by sequential treatment with MgCl₂ and CaCl₂ and then heat shock transformed with this construct. Transformants were selected on LB media containing chloramphenicol.

Transformed E. coli were then grown in LB with 30 μg/mL chloramphenicol at 37° C. 220 rpm, until OD₆₀₀ ˜0.6. IPTG 0.2 mM was then added and the cultures transferred to 28° C. with shaking at 220 rpm for a further 18 hours. Cells were harvested by centrifugation and kept at −20° C. before further use.

To test for bromoform production E. coli cells were resuspended in 1 mL water/100 mL culture volume and 20% v/v of this cell suspension was added to a solution of substrate listed below in Table 3, 100 mM HEPES (pH 7.4), 20 mM KBr, 1 mM Na₃VO₄, and 50 mM H₂O₂. Samples (100 μL) were incubated in sealed GC vials at room temperature for 24 hours. The head space of each sample was analysed by solid phase microextraction (SPME) GC/MS using a 7890A series gas chromatograph, a 5975C inert XL MSD mass selective detector and a MPS2 Gerstel multipurpose autosampler. Bromoform was identified in samples by comparison of the retention time with an authentic standard (Sigma, USA) and by mass spectral matching using the NIST/EPA/NIH Mass Spectral Library (version 2014). Relative bromoform quantities were measured by integration of the m/z=250 signal in extracted ion chromatograms generated using Agilent Mass Hunter software (version 10.0).

TABLE 3
Substrates tested for bromoform production with E. coli.
Substrates screened

No substrate control
1 mM ethanol
1 mM acetone
1 mM acetylacetone
1 mM hexanoic acid
0.15 mM palmitic acid + 1% (w/v)
NP40
1% (w/v) NP40 only

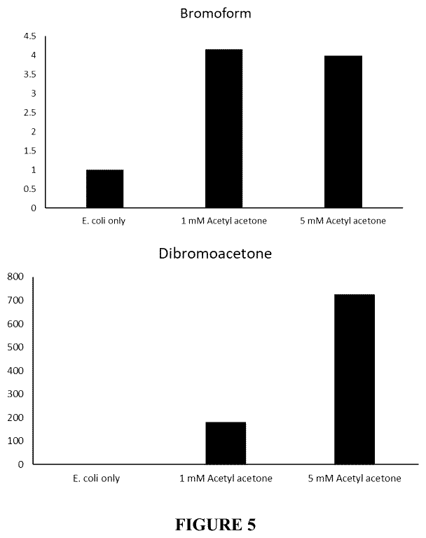

When comparing the tested substrates of Table 3 it was surprising that the addition of acetylacetone caused a dramatic increase (i.e. 10-fold) in bromoform production in E. co/i cultures compared with the other samples (FIG. 4). This result demonstrates the significant potential of acetylacetone for the synthesis of bromoform in relatively complex reactions, under physiological conditions.

To further investigate the potential for use of acetylacetone in complex mixtures, the possibility of adding it directly to culture samples was explored. VHPO was expressed in recombinant E. coli as described above, except the culture was also supplemented with 20 mM KBr and 0.5 mM Na₃VO₄. Three hours after induction of VHPO 0-, 1- or 5-mM acetylacetone and 100 mM H₂O₂ was added and incubation continued for 24 hours at 28° C. Relative bromoform, and dibromoacetone concentrations were assessed by SPME-GC/MS as above. Dibromoacetone was identified in samples by mass spectral matching using the NIST/EPA/NIH Mass Spectral Library (version 2014), relative quantities were measured by integration of the m/z=216 signal in extracted ion chromatograms generated using Agilent Mass Hunter software (version 10.0) (FIG. 5).

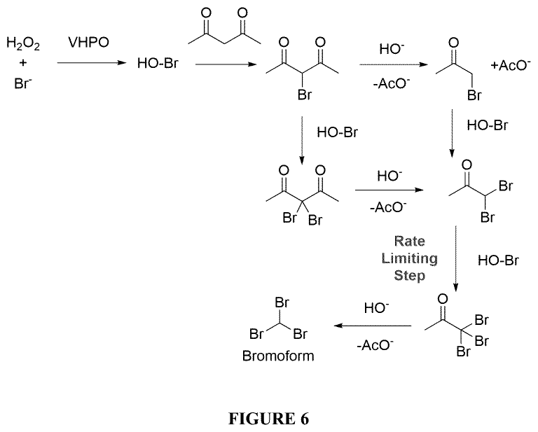

The accumulation of dibromoacetone is consistent with its rapid formation before the apparently rate limiting conversion of this intermediate to bromoform. This likely proceeds via the reaction scheme shown in FIG. 6.

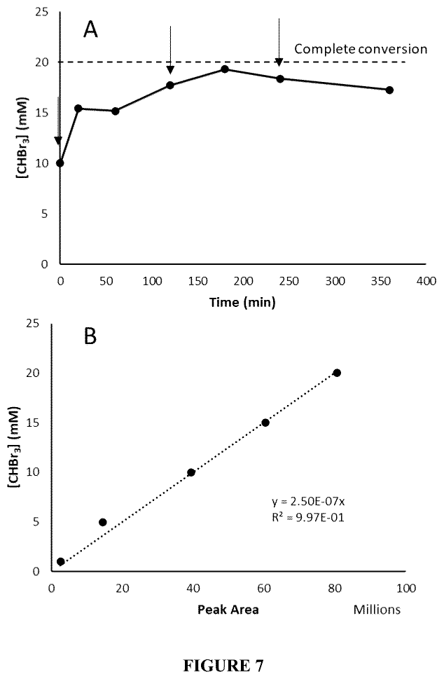

It was hypothesized that with sufficient hydrogen peroxide supply full conversion to bromoform could be achieved. To this end an additional reaction was set up with 10% E. coli cell suspension 100 mM HEPES (pH 7.4), 100 mM KBr, 1 mM Na₃VO₄, and 400 mM H₂O₂ added to initiate the reaction, then again after 2 and 4 hours. 10 μL of this reaction was sampled at 0, 20, 60, 120, 180, 240, and 360 minutes, the reaction was stopped by addition of 1 mL of ice cold brine and samples were stored in sealed GC vials at −20° C. before analysis by SPME-GC/MS as above. Bromoform was quantified by comparison with a standard curve prepared from authentic samples of bromoform, added to control samples (identical to the reaction, except containing no bromide), to account for matrix effects. The time course and standard curve is shown below in FIG. 7.

Approximately half the acetylacetone was converted rapidly to bromoform, indicating a high initial rate of reaction under these conditions. Starting material not already converted to bromoform was rapidly converted to dibromoacetone, consistent with previous experiments. After three hours near complete conversion to bromoform (97% yield based on acetylacetone) was observed. This required a significant excess of H₂O₂, likely due to catalase enzymes present in the cells. This result demonstrates that acetylacetone can be used for the rapid synthesis of large amounts of bromoform sufficient for further application (5 mg/mL). Preparations suitable for use as a livestock feed additive typically contain 1-4 mg/mL (Magnusson et al., 2020). Because VHPO enzymes release free HO—Br into solution they lack substrate specificity and it is expected that similar yields can be achieved with sufficient optimization of any system utilizing a VHPO enzyme for the synthesis of bromoform.

This increase in bromoform yield may be explained by the relatively low pKa of acetylacetone (Jones and Patal, 1974) compared to the other substrates, which corresponds to higher reactivity with HOBr generated by VHPO. This observation may also explain why bromination of dibromoacetone is rate limiting as this intermediate likely has a higher pKa than diketones shown in FIG. 6. To further clarify this effect, the experiment was repeated with substrates with that share the 1,3 dicarbonyl substructure with acetylacetone but have a range of pKa values (Table 4).

TABLE 4
Acetylacetone related substrates tested
for bromoform production with E. coli.

| Substrates screened | pKa (H₂O solvent reference) |
|---|---|
| No substrate control | — |
| 1 mM 5,5-dimethyl-1,3- | 5.2 (Brodwell, personal |
| cyclohexanedione | communication) |
| 1 mM acetyl acetone | 9.0 (Jones and Patal, 1974) |
| 1 mM 3,5-heptanedione | Not available |
| 1 mM ethyl acetoacetate | 10.7 (Brodwell, personal |
| | communication) |
| 1 mM acetone | 19.3 (Brodwell, personal |
| | communication) |

To test for bromoform production E. coli cells were resuspended in 1 mL water/100 mL culture volume and 20% v/v of this cell suspension was added to a solution of substrate listed in Table 4 in addition to 100 mM HEPES (pH 7.4), 20 mM KBr, 1 mM Na₃VO₄, and 100 mM H₂O₂. Samples (1 mL) were incubated in sealed tubes at room temperature for 24 hours. 10 μL samples from each were withdrawn and added to 1 mL brine in a GC vial before sealing. The head space of each sample was analysed by solid phase microextraction (SPME) GC/MS as above. Bromoform was quantified by comparison with a standard curve prepared from authentic samples of bromoform as described above.

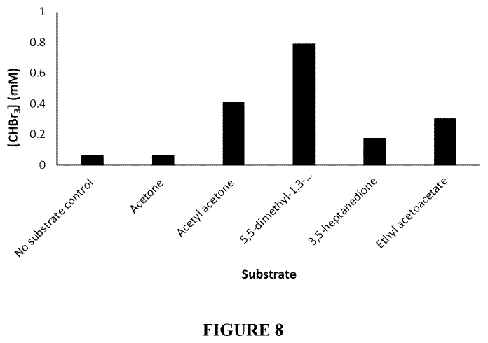

As shown in FIG. 8, 5,5-dimethyl-1,3-cyclohexanedione, acetylacetone, 3,5-heptanedione and ethyl acetoacetate each resulted in an increase in bromoform production, with bromoform yield seemingly increasing with decreasing pKa of the substrate used.

### Example 3—Screening of Transgenic Yeast Expressing the Acaryochloris marina and Asparagopsis taxiformis Haloperoxidase Genes with Potential Organic Intermediates for Increased Bromoform Production

The Asparagopsis taxiformis VHPO gene (Mbb1) encoding the A. taxiformis vanadium-dependent haloperoxidase (SEQ ID NO:2) was cloned by PCR from cDNA prepared from frozen A. taxiformis tissue. The Mbb1 ORF was cloned into the yeast expression vector pYES2 under the regulatory control of a Gal1 promoter and CYC1 terminator. S. cerevisiae strain InvSC1 was grown on YPD medium (1% w/v Bacto yeast extract, 2% w/v Bacto peptone, 2% w/v glucose with 1.5% w/v agar). A loop of cells was transformed using the LiAc/PEG transformation method (Gietz and Schiestl, 2007). Transformants were selected by growth on synthetic complete agar without uracil (SC-ura) with 2% w/v glucose.

The haloperoxidase gene from the marine cyanobacterium Acaryochloris marina MBIC11017 (Frank et al., 2016) (encoding SEQ ID NO:1) was synthesized by Epoch Life Science Inc. (Texas USA) using the published gene sequence (GenBank Accession CP000828-nucleotides 5020736-5018817). This gene sequence was codon optimized for S. cerevisiae expression (SEQ ID NO:18) and cloned into expression vector pYES2 under the regulatory control of a Gal1 promoter and CYC1 terminator. S. cerevisiae strain InvSC1 was grown on YPD medium (1% w/v Bacto yeast extract, 2% w/v Bacto peptone, 2% w/v glucose with 1.5% w/v agar). A loop of cells was transformed using the LiAc/PEG transformation method (Gietz and Schiestl, 2007). Transformants were selected by growth on synthetic complete agar without uracil (SC-ura) with 2% w/v glucose.

To induce expression of VHPO genes 15 mLs of Sc-Ura with 2% raffinose was inoculated with a single colony of transformed InvSC1 and incubated overnight (30° C., 220 rpm). Cells were harvested by centrifugation (1000 g, 10 minutes) and resuspended in 50 mLs Sc-Ura with 2% galactose and 1% raffinose at an OD600-0.4), and incubated for eight hours (30° C., 220 rpm). Cells were harvested by centrifugation (1000 g, 10 minutes) and stored at −20° C. before further use.

To test for bromoform production S. cerevisiae cells were resuspended in 1 mL water/100 mL culture volume and 20% v/v of this cell suspension was added to a solution of substrate listed above in Table 4 in addition to 100 mM HEPES (pH 7.4), 20 mM KBr, 1 mM Na₃VO₄, and 100 mM H₂O₂. Samples (1 mL) were incubated in sealed tubes at room temperature for 24 hours. 10 μL samples from each were withdrawn and added to 1 mL brine in a 20 mL GC vial before sealing. The head space of each sample was analysed by solid phase microextraction (SPME) GC/MS as above.

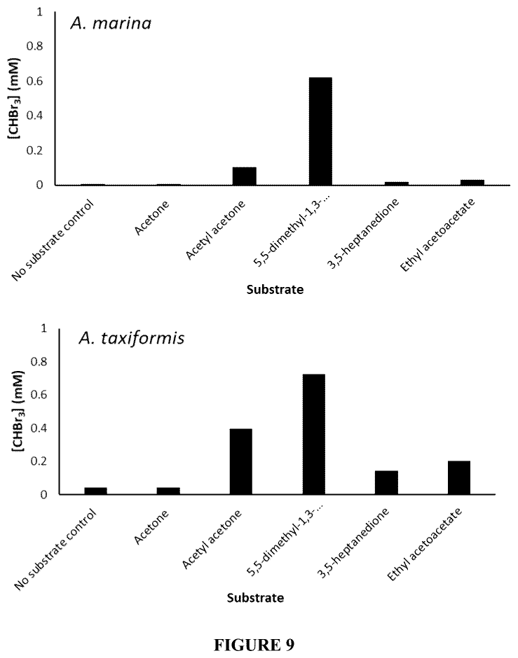

The bromoform yield increased with decreasing pKa of the substrate in both cases, demonstrating this effect is broadly applicable. As demonstrated in FIG. 9 this does not necessary limit the final yield of bromoform, but clearly contributes significantly to the rate of synthesis in these relatively complex sample mixtures.

### Example 4—Bromoform Synthesis by Extracted Enzyme

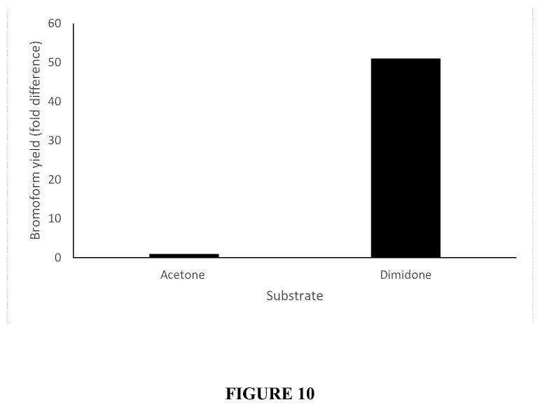

To determine the role of VHPO in the form of an extracted enzyme, 0.1 U of VHPO extracted from Corallina officinalis (Sigma) was added to 100 mM HEPES (pH 7.6), 1 mM Na₃VO₄, 50 mM KBr, 50 mM H₂O₂ and 10 mM acetone or dimedone. The whole reaction (100 μL) was transferred to a GC-Headspace vial and left at room temperature until analysis by SPME-GCMS as before. Relative bromoform levels were assessed as described above. Addition of dimedone gave a 51-fold increase in bromoform concentration relative to acetone (FIG. 10).

### Example 5—Bromoform Synthesis by E. coli

Having confirmed bromoform synthesis was significantly influenced by substrate pKa (Examples 2-3), the inventors turned their attention to identifying potential metabolites that could support the production of bromoform without disrupting core cellular functions. The biosynthesis of bromoform has not been definitively characterised, so a bromoform producing organism cannot be straightforwardly engineered by transplanting a cluster of genes from Asparagopsis to a suitable chassis organism. The 3-carboxyl thioesters that are intermediates of both fatty acid biosynthesis and catabolism have a similar pKa's to acetyl acetone (9.0).

This strategy utilizes tannic acid to inhibit FabG to promote the accumulation acetoacyl-acyl-carrier-proteins (Wu et al., 2010), alongside 3-amino-1,2,4-triazol to inhibit catalase and promote the accumulation of H₂O₂(Meir and Yagil, 1985). FabG is β-ketoacyl-ACP reductase [EC 1.1.1.100], an enzyme that catalyses the reduction of acetoacyl-acyl carrier proteins during fatty acid synthesis. Catalase consumes the intracellular H₂O₂ which is required for VHPO activity. Both inhibitors were added to cultures E. coli transformed to express VHPO upon induction with IPTG. The VHPO gene from the marine cyanobacterium Acaryochloris marina MBIC110174 was synthesized by Epoch Life Science Inc. (Tx, USA) using the published gene sequence (GenBank Accession CP000828-nucleotides 5020736-5018817). This gene sequence was codon optimized for E. coli expression and cloned into expression vector pACYCDuet1 under the regulatory control of a LacI repressor and T7 promoter and terminator sequences. Cell growth was allowed to continue for 18 hours in the presence of these molecules with the addition of KBr and Na₃VO₄, before harvesting the cells by centrifugation and analysis of bromoform levels.

Alongside IPTG (0.2 mM) the culture was also supplemented with 50 mM KBr, 1 mM Na₃VO₄, 10 mM 3-amino,1,2,4-triazole, and 0.5 g/L tannic acid or 0.1% oleic acid (emulsified with 1% tergitol (Merck, Ma USA)). Typically, 10 mL LB cultures were grown in 50 mL tubes with the lids closed. Vented tubes led to the loss of bromoform by evaporation. Cells were harvested by centrifugation and resuspended in 1 mL of brine (6 M NaCl), transferred into 20 mL headspace GC vials and sealed with a magnetic screw cap before analysis by SPME-GC-MS.

Inhibitors tannic acid and 3-amino-1,2,4-triazol were added along with KBr, Na₃VO₄ and IPTG to enable VHPO activity. After 18 hours bromoform could be observed directly by SPME-GC-MS of the E. coli cultures. Given the high hydrophobicity of bromoform the inventors reasoned that it would be concentrated in cell membranes and were able to further enhance the signal to noise ratio by centrifuging the culture and resuspending the cells in brine (6 M NaCl) before analysis. This procedure gave a clear signal corresponding to authentic bromoform standards (FIG. 11a). Bromoform was only observed when both inhibitors were added to the culture.

To further advance the hypothesis that any suitable 1,3-dicarbonyl could be converted to bromoform in vivo, the inventors targeted the 3-carboxyl thioester intermediate of β-oxidation. To encourage the formation of this metabolite the inventors supplemented the expression culture with 0.1% oleic acid and 1% tergitol to disperse the otherwise insoluble fatty acid in the culture. With the addition of IPTG, KBr, Na₃VO₄ and amino-triazole to inhibit catalase, the production of bromoform was again observed, albeit in only trace amounts (FIG. 11b).

Despite these apparently low yields we selected the di-carboxyl-intermediate from β-oxidation as a target for metabolic engineering in yeast, as this pathway is non-essential and entirely sequestered in the peroxisome. Placing bromoform synthesis in this organelle provides access to a potential source of H₂O₂ and may offer the cell some protection from the release of bromoform and the highly toxic HOBr.

### Example 6—Bromoform Synthesis by S. cerevisiae

A similar strategy was employed to produce bromoform from acetoacyl-CoA, where tannic acid was replaced with the fatty acid oleic acid to allow for the formation of acetoacyl-CoA by beta-oxidation.

As before, the VHPO gene from the marine cyanobacterium Acaryochloris marina MBIC110174 was synthesized by Epoch Life Science Inc. (Tx, USA) using the published gene sequence (GenBank Accession CP000828-nucleotides 5020736-5018817). This gene sequence was codon optimized for S. cerevisiae and cloned into expression vector pYES2 under the regulatory control of the Gal1 promoter. The DNA sequence encoding the ePST1 tag for localisation to the peroxisome (ttgggaagaggtaggcgctccaaactt) was later added to this gene using the Q5 site directed mutagenesis kit from New England biolabs (Ma, USA) according to the manufacturer's instructions. Primer sequences are given in Table 2. Subsequently, the sequence encoding VHPO-ePST1 was amplified by PCR and used with other PCR fragments from the pRS316 vector (Merck) and the pTEF2 promoter from BY4741 gDNA to construct the pRS-ePST1VHPO plasmid using the NEBuilder Hifi DNA assembly kit according to the manufacturer's instructions. The primers used were designed with the Gibson assembly wizard from Benchling (Ca, USA), and are given in Table 2. This plasmid was designed to allow the stable constitutive expression of VHPO by S. cerevisiae. The intended plasmid sequence was confirmed by Sanger sequencing. BY4741 cells were transformed by the lithium acetate method described by Gietz et al., (1992).

The coding sequences of both catalase genes (CTA1: YDR256C and CTT1: YGR088W) were deleted sequentially from the genome of S. cerevisiae BY4741 using the method detailed by Akada et al., (2006). Briefly, cells were transformed with a construct that replaced the target gene with a URA3 marker. The construct is designed such that a 40 bp repeat is present on either side of the marker after integration. Counterselection of the transformants on 5-fluoroorotic acid yields mutants with the URA3 marker excised from the genome by recombination between these two repeats, leaving no extraneous sequence in the genome. These mutants are uracil auxotrophs and can be transformed again with similar constructs targeting other parts of the genome to yield repeated deletions. Deletions were confirmed by PCR, as shown in FIG. 2. Primers used to generate constructs for this procedure are given in Table 2.

To promote the use of alternative media that promote β-oxidation a new plasmid was designed, pRS-ePST1VHPO, with a Tef2 promoter, which gives constitutive expression across a range of culture conditions. This plasmid was constructed by Gibson assembly from the eVHPOPST1 fragment with the cycl terminator from the pYES2 vector, the pTef2 sequence amplified from S. cerevisiae gDNA and the backbone of the pRS316 vector (Merck).

With this plasmid in hand, the media was adapted to allow for maximum flux through beta-oxidation, incorporating both oleic acid to induce β-oxidation and glycerol as a non-repressing carbon source. A small amount of glucose (0.1%) was also included to stimulate initial cell growth, alongside Mops buffer (pH 6.5), which improved bromoform production compared with unbuffered cultures. KBr and Na₃VO₄ were also added to allow for VHPO activity. Growth in this SDUGGO media, resulted in the production of bromoform (FIG. 12a-b). Once again bromoform could be detected in the cell pellet, only when both oleic acid and 3-amino-1,2,4-triazol were added to the culture consistent with the conversion of acetoacyl-CoA to bromoform in vivo.

These experiments can be conducted in Yarrowia. In particular, the experiments would include knocking out three catalase genes (YALI0F30987g, YALI0E34265g and YALI0E34749g) and inserting at least one copy of the VHPO gene into the genome, preferably using a CRISPR-Cas9 strategy.

These results confirm that bromoform can be synthesized in vivo from the 3-carboxyl thioester intermediate of β-oxidation within the peroxisome. Not only does this demonstrate the possibility of bromoform synthesis by precision fermentation, but also offers insights on the nature of the carbon substrate for bromoform synthesis in Asparagopsis species. Clearly, a suitably reactive carbon centered acid is required for the haloform reaction at physiological pH, likely this comes in the form of a 1,3-dicarbonyl. The exact nature of this carbon substrate, however, remains unknown. As this study shows, intermediates of both fatty acid catabolism and metabolism are suitable candidates for non-native biosynthesis, but a more bespoke substrate or process is likely responsible for bromoform synthesis in Asparagopsis, particularly considering the identification of 1, 1, 1, 5, 5, 5-hexibromo-2, 4-dione in A. taxiformis extracts.

It is envisaged that the cells used in these experiments could be used in the method of Example 1 or 2 to further promote the production of bromoform with extracellular provision of a 1-3-dicarbonyls defined herein.

### Example 7—Natural Resource for Controlled Production of Bromoform

Multiple publications have identified isolates of fungus with VHPO activity. For example Curvularia inaequalis are known to contain a vanadium chloroperoxidase with bromoperoxidase activity. Barnett et al (1997) investigated the activity of the VHPO from a Curvularia inaequalis obtained from the Central Bureau voor Schimmelculturen (CBS 102.42, CBS Baarn, The Netherlands). Applying the method to these fungi could provide a natural resource for controlled production of bromoform that can be used in or applied to a composition for dietary supplements for ruminants to reduce their methane production.

To apply the method described herein the inventors identified an isolate from The Queensland Plant Pathology Herbarium (BRIP) maintained by the Department of Agriculture and Fisheries, Queensland, Australia. To examine the enhanced bromoform production in Curvularia inaquaelis (Shear) Boedijn [BRIP 14448a] a live microbial culture was requested. The microbial sample can be grown in liquid minimal media (0.5% yeast extract and 0.2% mineral solution (4 mM K₂HPO₄, 2.5 mM CuSO₄, 2.75 mM FeSO₄, 4 mM MnCl₂, 5 mM ZnSO₄), the media can be supplemented with glucose between 1-10 g/L as described by Barnett et al. (1997). The isolate can be cultivated at 28° C. for 15 days after which the media can be supplemented with N-(2-Hydroxyethyl) piperazine-N′-(2-ethanesulfonic acid) (HEPES, 100 mM, pH 7.6), Na-₃VO₄, (0.1 mM), H₂O₂ (100 mM) and KBr (100 mM). To examine the increased bromoform production the dicarbonyl 20 mM of test substrate can be added. To compare the bromoform production an untreated control can be maintained without addition of a 1,3-dicarbonyl or with acetone. A further supplement of H₂O₂ (100 mM) can be added after 20 hours incubation at room temperature. Throughout the reaction 10 ul supernatant samples can be removed to assay the bromoform concentration using the GC-MS methods described herein.

The bromoform production in the presence of the 1,3-dicarbonyl is expected to be enhanced about 10 fold compared to the control. Bromoform yield will scale with the pKa of the substrate. The most dramatic difference is expected to be between control/acetone and dimidone, which could be 50-fold or more. It is expected the bromoform yield increases acetone<ethyl acetate<acetyl acetone<dimidone. To further optimise the bromoform production, catalase presence in the sample can be determined and an inhibitor supplemented to the incubation. Additional quantities of H₂O₂ (100 mM) can be added, for example 2, 3 or 4 additions of H₂O₂, on a daily schedule can be added. For most isolates 2 additions of H₂O₂ is sufficient but optimisation can be undertaken to evaluate the Curvularia's VHPO activity and catalase present in the sample.

It will be appreciated by those in the art that suitable fungal strains can be routinely screened using the established phenol red assay which confirms VHPO activity conveniently due to rapid bromination of phenol red to bromophenol blue. The assay can be conducted on whole cells or whole fungi media taken directly from cell culture as described by Hunter-Cevera and Sotos (1986). 100 μL of culture is centrifuged and cells resuspended in 200 μl of phenol red solution or the media is mixed 1:1 with phenol red solution. The phenol red solution contains a buffer e.g. 3-morpholinopropane-1-sulfonic acid (MOPS, 100 mM, pH 7.0) or HEPES (pH 7.4), KBr (20-50 mM), phenol red (0.45 mM), Na₃VO₄ (1 mM); if catalase is not present in the microorganism the phenol red solution may contain a low concentration 10 mM H₂O₂ or if catalase is present then a higher concentration of 100 mM is used. The reaction is initiated with the addition of 20 mM H₂O₂ and incubated at room temperature for 2 hours and 24 hours, respectively. VHPO activity is detected by the conversion of phenol red to bromophenol blue and this chromogenic substrate quantified by absorbance at 590 nm.

### Example 8—Expression of the Asparagopsis taxiformis VHPO Gene in Transgenic Plants

An Asparagopsis taxiformis VHPO gene construct (35S-AtVHPO) encoding all gene exons and introns and under the regulatory control of the cauliflower mosaic virus 35S promoter is transformed into Arabidopsis accession Columbia by Agrobacterium-mediated transformation and transgenic lines generated as described below.

Arabidopsis plants are grown under a 16 hour light, 21° C./8 hour dark, 18° C. growth regime in compost soil. An Agrobacterium GV301 strain is generated containing the 35S-AtVHPO gene in binary vector, vec8, which encodes hygromycin resistance. Upon emergence of flowers, plants are dipped in the Agrobacterium GV3101 solution (OD 0.8, 5% sucrose solution, 0.05% Silwet) for 2-3 seconds and then covered with a plastic cover for 24 hours. Upon maturity, seed are harvested from plants and sterilised by treatment with 70% ethanol followed by a 10% sodium hypochlorite solution and then rinsed 4 times with sterile distilled water. Seed is then plated then plated on solid Murashie and Skoog media (30 gm/L sucrose, 8 gm/L agar) containing 15 μg/ml of hygromycin B. Seedlings are grown under a 16 hour light/8 hour dark photoperiod regime and seedlings surviving selection then transferred to a soil compost mix and grown under glasshouse conditions. Putative transgenic lines are screened by PCR using primers specific for the 35S-AtVHPO transgene (Primer F—ATGACCGACACACAGAATCCC (SEQ ID NO:20); Primer R—CTAGATACGGATGGTCGAACCG (SEQ ID NO:21)). Positive lines possessing the VHPO transgene are identified.

To assay for VHPO activity leaf tissue is harvested from four independent transgenic lines, positive for the AtVHPO transgene, in addition to untransformed control Columbia plants. Tissues are ground in liquid nitrogen with 1M Na acetate buffer (pH 5.5) and supernatant collected after centrifugation at 15,000 g for 10 minutes. Polyvinylpyrrolidone (10% final) is added to the supernatant and the solution again centrifuged as described above.

These extracts (100 ul) are each added in triplicate to 100 μl of phenol red solution containing 3-morpholinopropane-1-sulfonic acid (MOPS, 100 mM, pH 7.0), KBr (50 mM), phenol red (0.45 mM), Na₃VO₄ (1 mM). The reaction is initiated with the addition of 20 mM H₂O₂ and incubated at room temperature for 2 hours and 24 hours, respectively. VHPO activity is detected by the conversion of phenol red to bromophenol blue and this chromogenic substrate quantified by absorbance at 590 nm.

To determine if these plant extracts can produce bromoform 1 ml of tissue extract from each sample is added to sealed GC-vials with KBr (20 mM), Na₃VO₄ (1 mM), Tris buffer (100 mM, pH 7.6) acetone (1 mM) and H₂O₂ (50 mM). These samples are incubated at room temperature for 1 week before analysis by GC-MS using solid-phase microextraction to monitor the presence of bromoform in the headspace. A single quadrupole GC/MS system (Agilent technologies, USA) with a 7890A series gas chromatograph, a 5975C inert XL MSD mass selective detector and a MPS2 Gerstel multipurpose sampler is used for bromoform detection in the headspace. Bromoform concentrations is calculated using a standard curve prepared from control samples with known amounts of bromoform added (Sigma, USA).

### Example 9—Inhibition of the Methanogenic Bacterium Species Methanobrevibacter smithii

Experiments can be undertaken to determine if supernatants from cell cultures, such transgenic expressing VHPO lines, e.g. the E. coli and yeast developed herein, or cells with endogenous VHPO, e.g. the known fungal isolates or cyanobacteria described could effectively suppress methane production. Methanobrevibacter smithii, a methanogenic bacterium found in the human intestine is capable of being used as a test culture equivalent rumen bacterial species. The experiment is designed with two media for cell culture growth, minimal media and minimal media+30 mM KBr. Bromoform would be expected to be produced only in the latter cultures due to the requirement of bromide for bromoform production. Acetylacetone is added to increase bromoform concentration in the final supernatant preparation.

Cultures are grown as described and then separated into two samples of equal weight. Each sample was supplemented with Na₃VO₄, (0.75 mM) acetylacetone (2 mM) and H₂O₂ (90 mM). KBr is added to one sample (minimal media+30 mM KBr) and omitted in the other (minimal media alone). Samples are gently stirred (50 rpm) at room temperature before adding a further 90 mM H₂O₂. After further incubation of up to one day the supernatant was separated from cellular components. Supernatant from bromide plus and minus samples was pooled separately. Triplicate tubes containing 9 mLs of BN medium are inoculated with an overnight culture of actively growing M. smithii for each treatment. At the same time supernatants from cell minimal media cultures and minimal media+30 mM KBr cultures are added to M. smithii culture tubes.

Ability to suppress methanogenesis from an actively growing culture of M. smithii cultures is assessed as follows. M. smithii cultures can be grown for 16 hours in BN media. Supernatant from cell cultures on minimal media or minimal media+30 mM KBr is added to the M. smithii culture. Each reaction is performed in triplicate.

Cultures were gassed to a pressure of 120 kPa with H₂ and grown in the dark at 39° C. with gentle shaking (50 rpm) for 16 hours. Gas pressures are recorded at 16 hours prior to addition of treatments and then at 22 and 40 hours (6 and 24 hours after treatment) after the addition of culture supernatants. Three mls of head space is removed and analysed by GC-MS to calculate methane concentration.

The concentration of bromoform produced by this in vivo culture with acetylacetone is sufficiently high to be used in as a feed supplement for a ruminant animal to effectively suppress methane emission from the rumen. Methods and devices for delivering such feed supplements to ruminant animals for effectively suppressing methane emission from the rumen are described in AU 2021221810 A1, the entire contents of which is herein incorporated in its entirety.

The present application claims priority from Australian provisional patent application AU2021901926, filed 25 Jun. 2021, the entire contents of which is incorporated herein by reference.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Akada et al. (2006) Yeast 23:399-405.
Barnett et al. (1997) Biochimica et Biophysica Acta (BBA)—Gene Structure and Expression 1352:73-84.
Bevan et al. (1983) Nucl. Acid Res. 11:369-385.
Certo et al. (2012) Nat Methods 8:941-943.
Doyon et al. (2010) Nat. Methods 7:459-460.
Frank et al. (2016) ChemBioChem, 17:2028-2032.
Franssen et al. (1988) Bioorganic Chemistry 16:352-363.
Gietz et al. (1992) Nucleic Acids Research 20:1425.
Gietz and Schiestl (2007) Nature Protocols 2:31-34.
Groenen et al. (1993) Mol. Microbiol. 10:1057-1065.
Haft et al. (2005) Computational Biology 1 (6):e60
Hunter-Cevera & Sotos (1986) Microbial Ecology, 12(1), 121-127.
Janssen et al. (2002) OMICS J. Integ. Biol. 6:23-33.
Jones and Patel (1974) Journal of the American Chemical Society 96:574-575.
Kim et al. (1996) Proc. Natl. Acad. Sci. USA 93:1156-1160.
Kim et al. (2012) Genome Res. 22:1327-1333.
Machado et al. (2016) Journal of Applied Phycology 28:3117-3126.
Magnusson et al. (2020). Algal Research, 51:102065.
Meir and Yagil (1985). Curr. Microbiol. 12:315-319.
Mojica et al. (2000) Mol. Microbiol. 36:244-246.
Needleman and Wunsch (1970) J. Mol Biol. 45:443-453.
Ohsawa et al. (2001) Photochemistry 58:683-692.
Tang et al. (2020) Metabolites 10:320.
Thapa et al. (2020) ACS Chemical Biology 15:1662-1670.
Thapa and Agarwal (2021) J. Physio. doi: 10.1111/jpy.13142.
Theiler et al. (1978) Science 202: 1094-1096.
van Embden et al. (2000) J. Bacteriol. 182:2393-2401.
Wu et al. (2010) Phytother. Res. 24:S35-S41.

1. A method of producing bromoform, the method comprising incubating an organism or part thereof, cells, a lysate of the organism or part thereof or cells, or a mixture thereof, or conditioned medium obtained from culturing a microorganism, cells, a lysate of the microorganism or cells, comprising a vanadate-dependent haloperoxidase, in the presence of at least one compound of Formula 1: 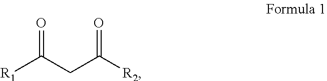
wherein R₁ and R₂ are:
independently selected from: hydrogen, hydroxyl, optionally substituted aliphatic, optionally substituted O-alkyl, or optionally substituted S-alkyl, or
R₁ and R₂ are joined to form an optionally substituted six membered ring of Formula 1a: 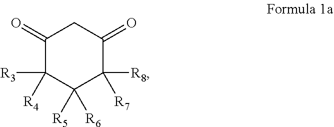
wherein R₃, R₄, R₅, R₆, R₇, and R₈ are each independently selected from hydrogen or an optionally substituted aliphatic.

2. The method of claim 1 which is performed in the presence of at least one or more or all of:
(i) a catalase activity inhibitor;
(ii) catalase gene(s) modification;
(iii) a compound that promotes the accumulation of acetoacyl-acyl-carrier-proteins;
(iv) a FabG or FAS gene modification;
(v) conditions that promote β-oxidation.

3. (canceled)

4. The method of claim 1 which comprises culturing the cells or microorganism.

5. The method of claim 1, wherein the organism is a plant, and the method comprises growing the plant.

6. (canceled)

7. (canceled)

8. The method of claim 1, wherein the microorganism is a bacteria, fungi or algae.

9-14. (canceled)

15. The method of claim 1, wherein the organism naturally comprises the vanadate-dependent haloperoxidase, preferably wherein the vanadate-dependent haloperoxidase is vanadium chloroperoxidase (VCPO) or vanadium bromoperoxidase (VBPO).

16. The method of claim 1, which further comprises harvesting the organism or part thereof, microorganism or cells.

17. The method of claim 1, which further comprises, following culturing, harvesting the medium.

18. A method of producing bromoform, the method comprising incubating a vanadate-dependent haloperoxidase in the presence of at least one compound of Formula 1: 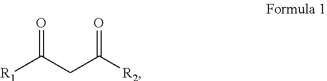
wherein R₁ and R₂ are:
independently selected from: hydrogen, hydroxyl, optionally substituted aliphatic, optionally substituted O-alkyl, or optionally substituted S-alkyl, or
R₁ and R₂ are joined to form an optionally substituted six membered ring of Formula 1a: 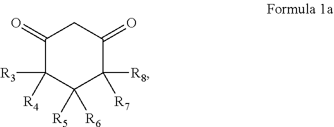
wherein R₃, R₄, R₅, R₆, R₇, and R₈ are each independently selected from hydrogen or an optionally substituted aliphatic, and wherein one or more or all of the following apply:
i) the vanadate-dependent haloperoxidase is not present in a protein extract obtained from an organism which naturally produces the vanadate-dependent haloperoxidase,
ii) the vanadate-dependent haloperoxidase is incubated with the at least one compound for greater than 90 minutes,
iii) the incubating occurs in the presence of hydrogen peroxide, and if hydrogen peroxide is added more than once, then at least two of the additions are greater than 10 minutes apart,
iv) the compound is not oxaloacetic acid nor acetylacetone.

19-21. (canceled)

22. The method of claim 2, wherein one or more of the following apply:
i) the compound that promotes the accumulation of acetoacyl-acyl-carrier-proteins is a FabG or FAS inhibitor, preferably tannic acid;
ii) the conditions that promote β-oxidation comprise incubating the organism, part thereof or cells thereof in the presence of a fatty acid as a carbon source, preferably a medium chain or long chain fatty acid, more preferably oleic acid;
iii) the catalase inhibitor is selected from the group consisting of 2,2′-azino-bis(3-ethylbenzthiazoline-6-sulfonic acid), 3-amino-1,2,4-triazole, 3-amino-4-hydroxybenzoic acid, azide, Ba²⁺, Co²⁺, Cu²⁺, EDTA, H₂O₂, KCl, MgCl₂, NaCl and nicotinic acid hydrazide, preferably 3-amino-1,2,4-triazole;
iv) the catalase, FabG and/or FAS gene(s) modification is a knock-down or knock-out, optionally wherein catalase is a knock down or knock out of both cytosolic and peroxisomal catalase genes.

23-26. (canceled)

27. The method of claim 1, wherein the at least one compound has a pKa of: 11 or less, 10 or less, 9 or less between 4 and 12, or between 5 and 10.7.

28. The method of claim 1, wherein the at least one compound is: 5,5-dimethyl-1,3-cyclohexanedione, acetylacetone, 3,5-heptanedione, ethyl acetoacetate, S-ethyl acetothioacetate, acetoacetyl coenzyme A or a mixture of any two or more or all thereof.

29. The method of claim 1, wherein the at least one compound is: 5,5-dimethyl-1,3-cyclohexanedione, 3,5-heptanedione, ethyl acetoacetate, S-ethyl acetothioacetate, acetoacetyl coenzyme A or a mixture of any two or more or all thereof.

30-36. (canceled)

37. The method of claim 1, wherein the incubating occurs in the presence of hydrogen peroxide.

38-41. (canceled)

42. The method of claim 1, which produces: at least 5, at least 6, at least 7, at least 8, at least 9 fold, at least 10, at least 15, at least 20, at least 25, at least 30, at least 35, at least 40, at least 45 or at least 50 fold more bromoform than a method performed under the same conditions in the absence of the at least one compound of Formula 1.

43. (canceled)

44. The method of claim 1, wherein the organism or part thereof, microorganism or cells comprise an exogenous polynucleotide encoding the vanadate-dependent haloperoxidase.

45. The method of claim 1, wherein the vanadate-dependent haloperoxidase comprises a sequence of amino acids provided in any one of SEQ ID NO's 1 to 8, or a sequence which is: at least 50%, at least 60%, at least 70%, at least 80%, at least 90% or at least 95% identical to any one or more or all of SEQ ID NO's 1 to 8.

46-49. (canceled)

50. Conditioned medium comprising bromoform obtained using the method of claim 1.

51. (canceled)

52. An extract or lysate of an organism or part thereof, microorganism or cells incubated in accordance with the method of claim 1, wherein the extract or lysate comprises bromoform.

53. A feedstuff, drink or animal feed supplement comprising bromoform produced using a method of claim 1 and at least one other feed, drink or supplement ingredient.

54-64. (canceled)


SEQUENCE LISTING (61 sequences)

SEQ ID NO: 1 - protein, 639 aa, Acaryochloris marina
MNTRRQQAQNIRNNAAELAANRPHPQHINNKEEYEYRRPKKDGNEPSHIANFTKGLPHDEHTGLLLNSADYDQFVLGIQSGDTTDFARTPLGPAELPKVHGCLSKQKIDCDDDHRSGFWKSQIAQGAAGGDGAKLRAWESAGAGLVFDLEGPDAQAVTMPPAPRLESPELTSEIAEVYSQALLRDIHFSQLRDPGLGDQVNACDSCPTQLSIYEAIDILNTVQIEGQNWFSANCCDLTDDEQARQRPLVTRQNIFRGIAPGDDVGPYLSQFLLIGNNALGGGVFGQEAGHIGYGAIRIDQRVRKATPCKDFMTNFETWLDVQNGADLRGLETYVDADPGKCREFPAYRVITTPRDLATYVHYDALYEAYLNACLILLGMGAPFDPGIPFQKPDVEDKQQGFAHFGGPQILTLVCEAATRGLKAVRFQKFNVHRRLRPEALGGLVDRYKHGKGAGDELKPVAALVEALENVGLLSKVVAHNQLQNQNLDRSGDPSSAGDNYFLPMAFPEGSPMHPSYGAGHATVAGACVTMLKAFFDHGWQLNLGMANGKYISYEPNQDGSSLQQVLLDCPLTVEGELNKIAANISIGRDWAGVHYFTDYIESLRLGEKIAIGILEEQKLTYGENFTMTVPLYDGGSIQI

SEQ ID NO: 2 - protein, 590 aa, Asparagopsis taxiformis
MTDTQNPNRAEVAFKVRVSAAELARARGSPTHLSNNSESRFRNPDGTRSLLANFTKGLPHIKETALVVSAIDYDNFVRAIDSGDPRDFADLPLGPQGVEPRFTSGIASDPEVGTRAWESGGAGLVFDLEGPDAQAVTMPPAPELDSDELVTEVTECYWMSLLRDVPFPTFESDPHIQSAAESINNTQWIKFKDSPPAHLTAAERSRLRGPITTANVFRGITPGDEVGPYLSQFLLVGTTGIANGNEVGDGFIQYGGMRMDQRVRVAKSHIDYMTTFGAYLDVQNAANVSGRELYNEEEPRFRFIHTPRDLATYVHFDALYQAYLNACIILLDIGAPFDSGIPFQLDNDIDKQQGFATFGGPHILSLVTEVATRALKAVRFQKFNVHRRLRPEAIGARVDRYCATKAPEFAGAAKLSEALDKKLLQKVHDHNKKQNLLSDRGNPRANDFNPDGDVSEGNLLMPMAFPEGSPMHPAYGAGHATVAGACVTVLKAFFDGGYRLPFCYITNEDGTGLQAVEIDEPLTVDGELNKICSNISIGRNWAGVHYFTDYIESIRIGEEIAIGILQEQKLTFSENFSMTLNKFDGSTIRI

SEQ ID NO: 3 - protein, 641 aa, Ascophyllum nodosum
MMKASGGIITALLAAFPPCLSQTVNELADMQTRPLLSGSVCRVRDTVDFLSPTKRAKITFKRRIGIAAGELAVGPTCHLNNGDEANVPLFDGQFHKSLPHDDVGRVNPEAYQLLLDCIESNDINVCDQVPSGVESDGRKLVNPLGGGGHQVDGADSDNIFIKQPDNLLSERLAAQQAEVYWMALLRDIPFSQFGTNNTVQMAAANLQGFDAFNGLSISRDADGNIDPMQDLFRTDWPGVSSGPMVSQFMLANFDIDGIVVEPKAKTLVPEMEYMTGVDTWLNIQNGGPPEDTLFVDEPLFIRNGRDLAALSFNDVLYTEAFRTILILFNESILAEAGPYGSSTRQEGFTTLGTSHYIHAMAAGSSSTRHAWYAKWQVHRVLRPEAYGGLLHFVINNLIDDVPLPASIVSNTELLNAVESLNQAQNGGTNQVFLLPMAVGEGSPVHPAYPSGHAINLGAYLTVLKAFLGFELGQRCFPSPMISNDAGTDRIPFVPSDGDRVGTCINEDGEEEVGLTYEGELNKVTSNVAIGRSHLGVHWRMDGVFGAEMGEAGAIRRLQQELGGLPEARDTEGPIPPASYKFRLYSGTMIELFPDNRYMLGDQMCKGFFTGDDFCVPADEEETAELEDLVLGSVVDQTCAEL

SEQ ID NO: 4 - protein, 656 aa, Chondrus crispus
MHLAALKLPHDIPSPAGTHRANSAKSLRDLCTDIARHRAHAASESNGEEADFASACYPTNFTKGLAHNNFGLVAQAEDYRVFVEAINSPAPGLFDSHVKSAEDREVAFKCRVKGAKPKWRGWESPRAGHVYELQGPDAGSLGMAPAPRVGSSELSAEMAEVYAMALLRDVPFKDICAGKSKEKFCEKDGDAAFSGTEIANLLNSIGLNQYERNRRFARTQEDTAPYVAPLSAQTLFRGSTPGALVGPYISQFMLIGARSIAGDGNGQSNFPAKEAAFDLNDGFIPYGSLIIDQRTISHKGCLDHMTNWAHWLDVQNGANLAKTDCWEPERRFITTPRDLATYVHFDALYEAYLNAALILIAMESPPSKGFPERSPSGRRTPFATFGGPHILSLVTEVATRCLKAVRRQKFNYHRRARPEVMGARLTLAEYIRQAIIEHNESQNRPDMVEMRAKCGLTCNDLHAFEGYDPKCNLLLPMAFPEGSPMHPAYGAGHATVAGGCVTILKAFFEMFEDCDAGKERELCDSKGMPIAFVPTSDGTKLMKDAKTKEPLTIQGELDKLAANISIGRNMAGVHYYSDYYDSLRMGERVAVGILLEQAPTYGDPVEMTFRSFDGDLIRVSGQAESAPTLTILDRNGSRVSVDDWWLRHVVGEEVIL

SEQ ID NO: 5 - protein, 598 aa, Corallina pilulifera
MGIPADNLQSRAKASFDTRVAAAELALNRGVVPSFANGEELLYRNPDPDNTDPSFIASFTKGLPHDDNGAIIDPDDFLAFVRAINSGDEKEIADLTLGPARDPETGLPIWRSDLANSLELEVRGWENSSAGLTFDLEGPDAQSIAMPPAPVLTSPELVAEIAELYLMALGREIEFSEFDSPKNAEYIQFAIDQLNGLEWFNTPAKLGDPPAEIRRRRGEVTVGNLFRGILPGSEVGPYLSQYIIVGSKQIGSATVGNKTLVSPNAADEFDGEIAYGSITISQRVRIATPGRDFMTDLKVFLDVQDAADFRGFESYEPGARLIRTIRDLATWVHFDALYEAYLNACLILLANGVPFDPNLPFQQEDKLDNQDVFVNFGSAHVLSLVTEVATRALKAVRYQKFNIHRRLRPEATGGLISVNKIAPQKGESIFPEVDLAVEELGDILEKAEISNRKQNIADGDPDPDPSFLLPMAFAEGSPFHPSYGSGHAVVAGACVTILKAFFDSGIEIDQVFEVDKDEDKLVKSSFKGTLTVAGELNKLADNIAIGRNMAGVHYFSDQFESLLLGEQVAIGILEEQSLTYGENFFFNLPKFDGTTIQI

SEQ ID NO: 6 - protein, 646 aa, Luminaria digitata
MKISSLGLTALLAAFAPCLGYEEPPEPTQPLLSGNVCRVRDSLDFLDPVPRAKVTLLKRLAIAKDEISVGPTCHINNGDEENVPLFAGQYHKTLPHDKFGQVDEDAYKKLLECVFTSDINECEKVPSGAGRRGGAKLTNPLGGTAHQVTGADSDNVFITTPDSLLSERLAAQQAEVYWMALLRDIPFGEFAKNDYVRLAAENLQGLPAFKGLNIPRSEGGKIDPVTDLFRTTWPGVTTGPVVSQFMLSDFLIDSIKVTPKADPLTPGVDYMTAFQPWLDVQNGASKLETTFDEENPRFIRNGRDLATIALRDQLYTEAFRAALILFTEGALGGEVGPYAEAERQQGFATFGEPHILTAMASASSVTRHAWYAKWQVHRMLRPEAYGALVHNTLMRDVITPLPDSILRNTELLNRVEVHNQRMNPDGEKTFLLPMAAAQGSPTHPAYPSGHAINNGAYITALKAFLGYEAGQKCFPNPVVSNDEGTKRIKYKPSGREIVGECVNEKGKLVEGLTYEGELNKISANVLLGRSHIGVHWRMDGVYGALMGETSCVRRLQQELPGLPEAREVEGKKRRGDIPPATYKFRLYSGKILELYGRNLYKLDGKLCEGAFTGDDFCDPIDEDDYSSFDDIVEEHAQFSLHGHTEL

SEQ ID NO: 7 - protein, 609 aa, Curvularia inaequalis
MGSVTPIPLPKIDEPEEYNTNYILFWNHVGLELNRVTHTVGGPLTGPPLSARALGMLHLAIHDAYFSICPPTDFTTFLSPDTENAAYRLPSPNGANDARQAVAGAALKMLSSLYMKPVEQPNPNPGANISDNAYAQLGLVLDRSVLEAPGGVDRESASFMFGEDVADVFFALLNDPRGASQEGYHPTPGRYKFDDEPTHPVVLIPVDPNNPNGPKMPFRQYHAPFYGKTTKRFATQSEHFLADPPGLRSNADETAEYDDAVRVAIAMGGAQALNSTKRSPWQTAQGLYWAYDGSNLIGTPPRFYNQIVRRIAVTYKKEEDLANSEVNNADFARLFALVDVACTDAGIFSWKEKWEFEFWRPLSGVRDDGRPDHGDPFWLTLGAPATNTNDIPFKPPFPAYPSGHATFGGAVFQMVRRYYNGRVGTWKDDEPDNIAIDMMISEELNGVNRDLRQPYDPTAPIEDQPGIVRTRIVRHFDSAWELMFENAISRIFLGVHWRFDAAAARDILIPTTTKDVYAVDNNGATVFQNVEDIRYTTRGTREDPEGLFPIGGVPLGIEIADEIFNNGLKPTPPEIQPMPQETPVQKPVGQQPVKGMWEEEQAPVVKEAP

SEQ ID NO: 8 - protein, 614 aa, Alternaria arborescens
MATFTPIPLPQIDEPAEYNTNYVLYWHHVGLELNRVTHTVGGPQTGPPISARALGMLQLAVHDAYFAIHPSSSFPTFLTSGADNPAYALPELSGADDARQAVAGASVTMLSMLYMKPPTNPNPNPGATISDNAYAQLQYVIDKSVTDAPGGVDAASSSFNFGKAVATVFFNLLFHAPGASQAGYHPTPGPYKFDDEPTHPVVLVPVDANNPDGPKRPFRQYHGPFYGKTAKRFATQTEHMIADPPAIRSAVGEQAEYDDSVRQIIAMGGATGLNSTKRSPFQTTQGMFWAYDGSNLVGTPPRFYNQIVRRIAVTYKKEEDLTNSEVNNADFVRLLALVNVACADAGIFSWKEKWEFEFWRPLSGVRDDNFRDPNRPDRGDPFWLTLGAPATNTNDIPFKPPFPAYPSGHATFGGAVFQMVRRYYNGRVGNWKDDEVDNIAIDMMVSEELNGLSRDLRQPYDPKAPITDQPGIVRTRVPRHFSSVWEMMFENAISRIFLGVHWRFDAAAAKDILIPTTTKDVYAVDNNGASLFQNVEDIRYTTMGTREGHDGLLPIGGVPLGIGIANEIFDTGLKPTPPEKQPVPPPPVNQSGPTKEMLEEAGSEEQVPMMDVAP

SEQ ID NO: 9 - DNA, 1920 bp, Acaryochloris marina
atgaatactaggcgacaacaagcccaaaacatccgcaataatgccgctgaattagctgctaatcgacctcacccacaacatataaataataaagaggaatacgaatatcgccgccctaaaaaagacggcaacgagcctagccacattgctaatttcaccaaggggttgcctcacgacgaacatacaggcttattactcaattccgccgactatgaccagttcgtcttaggcattcaatctggagacacgacggattttgctcgaaccccattaggtcctgccgaattacctaaagttcatggttgtttgagcaaacaaaagattgactgtgatgatgaccaccgttctggattctggaagtcacaaattgctcaaggagctgcgggtggtgatggagccaagcttagggcttgggaaagtgcgggggctggtttagtttttgacttagaaggtcccgatgcccaggcagtaacaatgccccctgctccgagattagaaagtccagagcttacttcagaaatcgcagaagtttattcacaagcattgctccgggacattcatttctctcaattacgagatcctggcttgggagatcaagtgaatgcctgtgattcttgcccaacccaactttctatttatgaagcaatagatattctaaatactgtccagattgaaggccaaaattggttctctgccaattgttgcgatctcacagatgatgagcaggctcgtcagcgaccactcgtaactcgacaaaatatctttcgtggcatcgcacctggagatgatgtcggtccctatctatcccagtttctgctgattggtaataatgcgctgggaggtggggtatttgggcaagaagccggccatatcggatatggtgcaatacgaattgatcagcgagtcagaaaggctaccccctgcaaagacttcatgactaacttcgaaacttggttagatgtgcaaaatggtgcagatttgcgaggtctagaaacctacgttgacgctgatccgggaaaatgtcgtgaattcccagcctatcgagtcatcactacacctcgggacttagcaacctatgttcactacgatgcactatatgaagcttatttaaacgcttgcttgattttgctgggtatgggagctcctttcgaccccggtattccgttccaaaaacctgatgtggaagataaacagcaagggtttgcccactttggaggaccgcaaatcttaaccctagtttgtgaggccgcaacaagagggttaaaagcagttcgtttccaaaagttcaatgttcatcgtcgtttacgccctgaagctctaggtggtttagttgatcgctataagcatggtaaaggtgcgggtgatgaacttaaacccgttgcagccttagttgaagcgctagagaatgttggtctcctcagcaaagttgtagcccataatcaactccaaaatcagaatcttgaccgcagtggcgatccttccagcgcaggggataattacttcctaccaatggccttcccagaaggatctcctatgcaccctagttatggtgcaggacatgcaactgtggctggagcttgtgtcactatgcttaaagctttctttgatcatggttggcaactgaatttgggtatggcgaatgggaaatacatatcctacgaacctaatcaagatgggagcagtcttcaacaagttcttttagattgccctctcacagtggaaggtgagttgaataagattgcagctaacatctctattggacgggattgggctggcgtgcattacttcaccgattacatcgaatccttacgattaggcgaaaagattgcgatcggcattttagaagagcaaaagctgacctacggagagaactttacaatgaccgttcccctctatgatgggggcagcattcaaatctag

SEQ ID NO: 10 - DNA, 1773 bp, Asparagopsis taxiformis
atgaccgacacacagaatcccaatcgtgcagaagtggccttcaaagtccgcgtatcagcagctgaactggcacgcgctcgtggctccccaactcatctgtccaacaactcagagagtcgtttcagaaaccctgatggtacccgttccctgctcgccaatttcaccaagggtctcccgcatatcaaggaaaccgctctagtggtgtccgcaatcgactacgataactttgttcgcgcaattgacagcggtgatccccgtgatttcgcagacctgccgctcggtccgcagggtgttgagcccagattcacctccggtatcgctagcgacccagaggtcggaacccgtgcctgggaaagtggtggtgctggtcttgtcttcgatctcgaaggtcccgatgctcaggctgtcactatgccgcccgcccccgagctggacagcgatgaattggttactgaagtcacagaatgctactggatgtctctgctgagagacgttcccttcccgacatttgagagcgatccccatattcagtcagctgcagagtccatcaataacacccaatggattaaattcaaggacagcccaccagcacatctcacagctgctgagcgttcccgtctcagaggccccatcaccactgccaacgtcttcagaggtatcacaccgggagacgaagtcggaccctatctgtcacagtttctcctggtcggtacgaccggtattgccaatggcaatgaagttggcgatggattcatccagtacggtggcatgcgtatggaccagcgcgtgcgtgtcgccaagtcacacattgattacatgaccacattcggcgcctatctcgacgtgcaaaatgccgccaacgtctccggtcgcgaattgtacaatgaagaggaaccccgtttccgcttcattcacacccctcgtgatctggccacttatgtgcatttcgacgccttgtatcaggcctacctcaacgcttgcatcatcttgctcgacatcggtgcgccattcgactctggaattcccttccagctcgataacgacattgataagcagcaaggatttgccactttcggcggtccgcacatcctctctctagttaccgaagtggccacccgagccctcaaggctgttcgcttccagaaattcaacgtccatcgtcgtctgcgtccggaggccattggtgcccgagtcgatcgatactgcgcaacaaaggcacccgaatttgctggcgctgccaagctctccgaagcgctggacaagaagttgttgcagaaggtgcacgatcataacaagaaacaaaacttgttgagcgatcgaggaaacccgcgtgcgaacgacttcaaccccgacggagatgtgagtgaaggtaatctgctgatgcccatggcgttcccagagggaagtcctatgcatcccgcgtacggcgctggacatgcgactgttgcaggcgcttgtgtcacagtgttgaaggcgttcttcgatggtggttacaggttgccgttctgttacatcaccaacgaggatggaacaggccttcaggcagtggaaattgacgaaccgttgacggtcgacggcgagctgaataagatttgctccaatatttcgattggtcgcaattgggctggcgtgcattacttcactgattacatcgagagtattcgcattggtgaggagattgccattggtattctgcaggagcaaaagctgacgttcagtgaaaacttctcaatgacactgaacaagtttgacggttcgaccatccgtatctag

SEQ ID NO: 11 - DNA, 1926 bp, Ascophyllum nodosum
atgatgaaggcttctggcggtatcatcaccgctcttctggcggcatttcccccgtgcctcagccagactgtcaacgagcttgcagacatgcagacgcggccgctcttgagcggtagcgtgtgccgtgtcagggacaccgtcgacttcctcagccccaccaagcgcgcgaagattaccttcaagaggcgcattggaatcgccgcgggagagctcgccgtggggcccacgtgccacctcaacaacggcgatgaggcgaacgtccccttgttcgatggacagttccacaagtcccttccgcacgacgacgtcggacgggtgaatcccgaggcctaccagctgctactcgactgcatcgagtccaacgacatcaacgtgtgcgatcaggtaccttcgggggttgagagcgatggtcgcaagctcgtgaaccccctcggaggcggtgggcaccaggtggacggtgctgatagcgacaatatcttcatcaaacagcccgacaaccttctctccgaacggctggccgctcagcaggctgaggtgtactggatggcccttctcagggacatcccgttctcccagttcggcacaaacaacaccgtacagatggccgcagcaaaccttcagggtttcgacgccttcaacggactcagcatctccagggacgcggacggcaacatcgaccctatgcaagacttgttcaggactgactggccgggcgtgagttctgggcctatggtgtcgcagtttatgctggcgaacttcgacatcgatggtatcgtcgtggagccgaaggcgaaaactctcgtgcccgaaatggagtacatgacaggggtggacacgtggcttaatatacagaacggtggaccaccggaggacaccctctttgtcgacgaaccactcttcatccgcaacggccgtgatctcgctgctctctccttcaacgatgttctctacaccgaggccttccgcacgattctcatactgttcaacgaaagtatcctcgccgaagctggcccgtacggatcttccacgcggcaggaaggattcacgaccttggggacatctcactacatccacgccatggccgccggaagctcctctacccgccacgcttggtacgccaagtggcaagtacaccgtgtcctcaggcccgaggcctacggcggcttgttgcacttcgtgattaacaatttgatcgacgacgtcccgctgcctgcgtcgatcgtcagcaacacggagctcctcaacgccgtggagagtctcaaccaggcgcagaacggcggcaccaaccaagtcttccttctcccgatggctgttggcgagggttcgcctgtgcaccccgcataccctagcggccacgcgatcaaccttggcgcttacctcaccgtgctcaaggcattcctcggcttcgagcttgggcagaggtgcttccccagcccgatgatctcaaacgatgccggtacggatcgcatcccgttcgtaccgtctgacggagatcgcgtgggtacctgcatcaacgaagacggagaagaggaggtcgggctcacgtacgagggcgagctcaacaaggtgacctcaaacgttgcgattggccgctcgcaccttggagtccactggcgcatggatggcgtcttcggcgccgagatgggcgaggccggcgccatccgccgccttcagcaggagcttggcggtcttcctgaggctagagacacagaagggcctatccctccggcctcatacaagttccgcctctacagcggcacgatgatcgagctgttccccgacaaccgctacatgctcggagaccagatgtgcaagggcttcttcaccggcgatgacttctgcgtcccggcggatgaagaagagaccgctgagttggaggacctcgtccttggttcggtcgtcgaccaaacctgcgccgagctctaa

SEQ ID NO: 12 - DNA, 1971 bp, Chondrus crispus
atgcatttagctgctttgaaactaccccatgatattccatccccggctgggacacatcgcgcaaactctgccaaatcactgcgagatttgtgtacagacattgctcgacatcgtgcacacgcagcctcagaaagtaacggggaagaggccgactttgcatcagcctgctatcccacgaactttacgaaagggttagcccataacaactttggtctggttgctcaagctgaggactaccgtgtctttgtcgaggcgattaattcccctgctccaggcctatttgatagccatgtaaagagcgcagaagatcgagaggtggcatttaaatgcagagtaaagggagcgaaaccaaagtggcggggctgggaaagtccgcgcgctgggcatgtctacgaattacagggcccagatgctggctctttggggatggcacccgcaccgcgggtgggatcatcggaattgtcggcagagatggcagaagtgtatgcgatggcattgctgcgcgatgtgccgtttaaggatatctgcgctggcaaaagcaaagagaagttttgtgaaaaagatggcgatgcggcattctcgggtacagaaatagcgaacctgctcaactcgatcgggctaaatcaatacgagcgcaaccgtcgatttgcacgcacgcaggaagataccgcaccgtatgttgcaccactaagtgcgcaaacactttttcgtggatcgacacccggtgctctggttggcccgtatatttcgcagtttatgttgatcggagcgcgctctattgctggcgatggcaacggccagtcgaactttcccgcaaaggaagcagcgtttgacttgaatgatggctttattccttacggctcactgatcatcgaccaaagaacgatctcccataaggggtgcttggatcacatgaccaactgggctcattggctggatgtccaaaatggggccaacctagcgaaaactgactgctgggagcccgagcgaaggtttatcacaacaccgcgagatttagccacttacgttcactttgatgccctttatgaagcgtatctcaatgcagctttaatcctgatagccatggagtcgccaccctcgaaaggatttcctgaacggagtccatcagggcgtcggacaccctttgcgacgtttggagggccgcatatcttgtcgctggtgacggaggtggctacccggtgcttaaaggcagtgaggcgtcagaagttcaactaccaccgtcgtgctagacctgaagtcatgggggcgcgcctaacgctagccgaatatatccgtcaggcaattattgagcacaacgagtcgcaaaaccgtcccgatatggttgagatgcgagccaaatgcggtctgacttgcaacgatctgcacgcttttgagggctacgatcccaagtgcaatttgctgctgcctatggctttcccagaaggctcgcccatgcatccagcctacggtgcggggcatgccacggtggcaggcgggtgcgtgacgatcctaaaggccttctttgagatgttcgaagactgtgatgccggcaaagaacgggagttgtgtgattcgaaaggaatgccgatcgccttcgtccccaccagcgacggcaccaagctcatgaaggacgccaagactaaagagccattaactatccagggtgagctcgacaaactggcggccaatatttcgatcggtcgcaatatggcgggagtgcactattactctgactattacgattcactacgaatgggcgaacgagttgctgttggcatcctattggagcaagcaccgacctatggcgatcccgtggagatgacatttaggagcttcgacggcgatcttattcgggtatcgggccaagctgagagtgcgccaacactcaccattttggatcggaatggctcacgcgtttctgtagacgactggtggctacgtcatgtggtgggagaggaagtcatattgtag

SEQ ID NO: 13 - DNA, 1803 bp, Corallina pilulifera
atgggtattccagctgacaacctccaaagtcgcgccaaggcttcattcgatacgcgtgtagctgcggccgagcttgcactcaaccgcggagttgtaccatcgtttgcaaatggggaagagcttctctaccgcaacccggaccctgacaacactgatccgagcttcatcgctagcttcacaaagggtcttccgcatgacgacaatggcgctattatcgaccccgacgacttcttggccttcgttcgtgcaatcaatagtggcgatgaaaaggagatcgccgacctcacattggggccagctcgcgacccggagactggcttaccaatctggcgctcggatctggcgaattctctcgagctcgaagtgcgaggatgggagaacagctctgccggtctcaccttcgacctggagggcccggacgcgcagtcgattgccatgccaccggcgcctgtgctcacgagccctgagctcgtcgccgagatagcagagctgtacctgatggcgcttggacgcgaaatcgagttcagcgagtttgattccccaaagaacgcagagtatattcagtttgctattgatcagcttaacgggctggagtggttcaacacaccggcaaagctcggagatccgcctgcggaaatccgtcgccgtcgcggtgaggtgactgttggaaacttgttccgcggtattcttccaggctctgaggtcggcccgtacctcagccagtacatcatcgttggtagcaagcagattggctcagcgacagttggtaacaaaactctcgtgagccccaatgctgctgatgagtttgatggtgaaatcgcctacggaagcatcaccattagccagcgtgtgcgtatcgccacgcctggacgcgacttcatgaccgacttgaaggtattccttgacgtccaggacgctgcggacttccgaggctttgagtcgtatgagccgggagcacgcctcatccggacgatccgcgatcttgcgacgtgggtgcactttgacgcactgtacgaggcctacctcaatgcgtgcctaattctgttggcgaacggcgtgccgttcgatcccaaccttccgttccagcaggaggacaagctcgataaccaggacgtgtttgtgaacttcggatccgcacacgtgctgagtctggtgactgaagtggctacgcgcgcgttgaaggcggtacggtaccagaagtttaacattcatcgtcgcctgcgccctgaggctaccggtggtctgattagcgttaacaaaatcgcaccgcagaagggcgagagcattttccctgaggttgatcttgctgttgaagagcttggagatatcttggagaaagctgaaattagcaataggaagcagaacatagctgacggagatcctgaccctgatccttcattcctgttgccgatggcattcgccgagggcagcccattccatccgtcctacggaagcggccacgctgtggttgctggcgcatgtgtgacgatcctgaaggcgttcttcgactccggcatcgagatcgatcaggtgttcgaggtcgacaaagatgaggacaagcttgtgaagtcgtctttcaagggaactctcactgttgccggtgaattgaacaagctcgccgacaatattgcgatcgggcgtaacatggcaggtgttcactacttctctgaccagttcgagtcacttctgctcggtgagcaggttgcgattggaatcttggaagagcaaagtctgacgtatggcgagaacttcttcttcaacttgccgaagtttgatggaactacaatccagatctaattttaa

SEQ ID NO: 14 - DNA, 1941 bp, Laminaria digitata
atgaagatttcttccttgggcttgaccgctttgctggcggcgttcgctccctgcctcggctacgaggagcctcccgagcccacgcagcctctgctcagcgggaacgtgtgcagggtgcgagactccctcgacttccttgacccggtgccgcgcgcgaaggtgaccctgctcaagcgcctggccatcgccaaggacgagatatcggttggacccacttgccacatcaacaacggcgacgaggaaaacgtcccgctcttcgctgggcagtaccacaagacgctaccgcacgacaagttcggtcaggtggacgaagacgcatacaagaagctgctggagtgcgtcttcacgagcgacatcaacgagtgcgagaaggtgccctccggcgccggccgaagaggtggcgcgaagctgaccaaccccctcgggggcaccgcgcaccaggtcaccggtgctgacagcgacaatgtgttcatcaccacgcccgacagtctcctctccgaaaggctggccgcgcagcaggcagaagtttactggatggccctgcttagggacatccccttcggggagttcgccaagaacgactacgtcaggctcgccgcagagaacctgcagggcctgccggcgttcaagggactcaacattccccggagcgagggaggaaagatcgaccctgtcactgatctgttccgaacgacctggccgggtgtgaccaccggtcccgtcgtgtctcagttcatgctgtcggacttcctgatcgactctatcaaggtcaccccgaaggctgacccccttaccccaggggtggactacatgaccgccttccagccgtggctcgacgtacagaacggggcttccaagcttgaaactactttcgacgaggagaacccccgcttcatccgcaacggccgagacctggccactatcgccttgagggatcaactctacacggaggccttccgcgccgcgctcatcctgttcacggaaggtgcacttggcggcgaggtcgggccttacgcggaggccgagcgccagcaggggttcgccactttcggggagccgcacatcctcaccgccatggcctcggccagctctgtgacgcgccacgcgtggtacgctaagtggcaagtccaccgcatgctgcgacccgaggcgtacggtgccctggtacataacacgctcatgagggacgtcatcaccccgctgccggactctatcctgaggaacacggagctcctcaaccgagtggaggtgcacaaccagcgtatgaacccagacggcgagaagaccttcctgctccccatggcggcagcccagggctctcccacgcaccccgcttaccccagtggacacgccatcaacaacggcgcctacatcaccgcgctcaaggcgttcctcgggtacgaggccggccagaagtgcttccccaaccccgtggtctcgaacgacgagggaaccaagaggatcaaatacaagccctccggaagggagattgtgggcgagtgcgtcaacgagaagggcaagctcgtcgaaggcctcacgtacgagggagagctcaacaagatcagcgccaacgtgctcttgggaaggtcccacatcggtgttcactggaggatggacggcgtgtacggagcgctcatgggagagaccagctgcgtccgccgcctgcagcaggaacttcctggcctcccggaggcccgcgaggttgagggcaagaagcgccgtggtgacatccccccggcgacttacaaattccgcctttacagcggtaagattcttgagctctacggcaggaacttgtacaagctggacggcaagctctgcgagggggccttcaccggcgacgacttctgtgacccgatcgacgaggacgactacagctcgttcgacgacatcgtcgaggaacacgcccagttctcgctccacggacacaccgagctctaa

SEQ ID NO: 15 - DNA, 1830 bp, Curvularia inaequalis
atggggtccgttacacccatcccactccctaagatcgatgaacccgaagagtacaacaccaactacatactattctggaaccatgtcggtttggaactcaaccgcgtaactcacactgttggaggccccctgacgggaccacctctctctgccagggctctgggtatgctgcacttggctattcacgacgcctacttttctatctgccctccgaccgacttcaccaccttcctctcacctgatactgagaatgccgcgtaccgtctacctagccctaatggtgcaaatgatgctcgccaagcagtcgctggagctgccctcaagatgctgtcttcactgtacatgaagcccgtcgagcagcctaaccctaaccccggcgccaacatctccgacaacgcttatgctcagcttggcttggttctcgaccgatcagttctggaggcacctggtggcgtggaccgagagtcagccagtttcatgtttggtgaggatgtagccgatgtcttcttcgcactcctcaacgatcctcgaggtgcttcgcaggagggctaccaccctacacccggccgctataaatttgacgatgaacctactcaccctgtcgtcctcattccagtagaccccaacaaccctaatggtcccaagatgcctttccgtcagtaccacgccccattctacggcaagaccacgaagcgttttgctacgcagagcgagcacttcctggccgacccaccgggcctgcgttctaatgcggacgagaccgcggagtatgacgacgccgtccgcgtcgctatcgccatgggtggtgctcaggctctcaactccaccaagcgtagcccatggcagacagcacagggcctatactgggcctacgatgggtcaaacctcattggcacaccacctcgcttttacaaccagatcgtacgtcgcatcgcagttacgtacaagaaggaagaggaccttgccaacagcgaagtcaacaatgcggatttcgcccgcctcttcgccctcgtcgacgtcgcttgcacagacgctggtatcttttcctggaaggagaaatgggagttcgaattctggcgcccactatctggtgtgcgagacgacggccgtccagaccatggagatcctttctggctcactctcggtgccccagctactaacaccaacgacattccattcaagcctcctttcccagcttacccatctggtcacgcgacctttggcggtgctgtgttccaaatggtgcgtcgatactacaacggccgcgtaggtacatggaaggacgacgaacccgacaacattgccatcgatatgatgatctcggaggagctcaacggcgtgaaccgcgacctacgccagccttatgaccccacggccccaatcgaagaccaacccggtatcgtgcgcacccgcattgttcgccacttcgactcggcctgggaactcatgttcgaaaacgccatttcgcgcatcttcctcggtgtccactggcgtttcgatgccgccgccgcccgcgacattctcattcccacgacgacaaaggacgtctacgctgtcgacaacaatggcgccaccgtgttccagaacgtagaggacattaggtacacaaccaggggtacgcgtgaggaccccgagggcctcttccctatcggtggtgtgccactgggtatcgagattgcggatgagatttttaataatggacttaagcctacgcccccggagatccagcctatgccgcaggagacaccggtgcagaagccggtgggacagcagccggttaagggcatgtgggaggaagagcaggcgccggtagtcaaggaggcgccgtag

SEQ ID NO: 16 - DNA, 1845 bp, Alternaria arborescens
atggcaacattcacccctatcccgcttcctcaaattgatgaaccggcagagtacaacacgaactacgttctgtattggcaccatgtcggcttggagcttaaccgcgtcacacataccgtcggaggcccgcagactggcccaccaatctctgctagagctctgggcatgctccagttggctgtacatgatgcctacttcgccatccacccctcttctagcttcccaacctttttgacatctggcgccgacaaccctgcctacgctctgcccgagttgagcggcgcggacgatgcccgccaggcggtagctggtgcatctgttactatgctgtctatgctttacatgaagccccctaccaaccccaaccccaatcctggcgccaccatttccgacaacgcctatgcacagcttcagtatgtcattgacaaatcagtaaccgatgcacccggtggtgtagatgccgcgtccagcagtttcaacttcggaaaggcagtagctactgtcttcttcaacctactcttccacgccccaggtgcctcgcaagctggctaccaccccacacctggcccatacaagttcgacgatgagcccactcaccccgtcgtccttgttcccgttgacgcaaacaacccggatggtcccaagcggcctttccgccagtatcacggcccgttctatggcaagactgctaagcgttttgctacacagactgagcatatgattgctgacccaccagctattcgttctgccgttggtgagcaagctgaatacgatgatagtgttcgtcaaatcattgccatgggtggagctaccggtctcaactccaccaagcgcagccctttccagacaactcaaggcatgttctgggcctacgatggctccaacttggtcggcacaccacccagattttacaaccagattgtccgccgcatcgcagtgacgtacaagaaggaggaagacttgactaacagcgaagtcaacaacgcagactttgtccgtctccttgctctggtcaacgtagcctgtgccgatgcaggaatcttctcctggaaagagaagtgggaatttgaattctggcgtccactctctggtgttcgtgacgacaacttccgcgacccaaatcgcccagatcgtggcgaccctttctggcttactctcggcgccccagccacaaacacaaacgatatccctttcaaaccccccttccccgcttacccctctggtcacgccacattcggcggcgctgtcttccagatggtccgccgctactacaatgggcgagttggaaactggaaggacgacgaagtggacaacatcgccatcgatatgatggtatccgaggagctcaacgggctgagccgtgatctccgccagccctacgaccccaaagcgcccattactgatcagccaggtatcgtgcgcacacgtgttccacgccacttctcttccgtctgggagatgatgttcgagaacgcaatctcgcgtatctttctcggtgttcactggcgcttcgatgctgcagccgccaaggatattttgatccccacgacgacaaaggatgtctacgccgtagacaacaacggcgcttccttgttccaaaatgtcgaggatatccgttatacgactatggggactagggagggtcacgatgggcttctgccgattggtggtgtgccgcttggtattgggattgcaaatgagatctttgatacaggtctcaagcctaccccaccggagaaacagccagtgccgccgcctccagtcaaccagagcggaccgacgaaggagatgttggaggaagcgggaagtgaggagcaggtccctatgatggatgttgcaccctga

SEQ ID NO: 17 - DNA, 1920 bp, Artificial Sequence
  Open reading frame encoding Acaryochloris marina vanadium-dependent haloperoxidase codon optimised for expression in E. coli
atgaacactcgtcgtcagcaggcacagaacatccgtaataatgcagccgaactggctgcgaatcgtccacacccgcagcatatcaacaacaaagaggaatacgaatatcgtcgtccgaaaaaagacggcaatgaaccgagccacatcgctaacttcactaagggcctgccgcacgatgaacatactggtctgctgctgaactctgcggactacgaccaattcgttctgggtattcagagcggtgacactacggattttgcacgtacgccactgggtccggctgaactgcctaaagtacacggttgtctgtctaaacaaaaaattgattgcgatgatgatcatcgttccggcttctggaaatctcagatcgcgcaaggtgcagcaggtggtgatggtgctaaactgcgtgcttgggagagcgcaggtgctggtctggtcttcgacctggaaggtccagacgcacaggcagtaaccatgccaccggctccacgtctggaatcccctgaactgactagcgagatcgcagaagtgtactcccaggctctgctgcgtgatattcatttctcccagctgcgtgatccgggtctgggtgatcaggtgaacgcttgcgactcctgcccgactcagctgagcatctatgaagctatcgacattctgaacaccgtacagatcgaaggtcagaactggttcagcgctaattgttgcgatctgaccgatgatgaacaggcacgtcagcgtccgctggtaacccgtcagaacattttccgtggtatcgcgccaggcgatgatgttggtccgtacctgtctcagttcctgctgatcggtaacaacgcactgggcggtggcgtattcggtcaggaagctggtcatatcggttacggtgcaattcgtatcgatcaacgcgtccgtaaagcaaccccgtgcaaggactttatgaccaacttcgaaacttggctggacgttcagaacggcgctgacctgcgtggcctggaaacgtatgtggatgctgatccgggcaaatgccgtgagttcccggcataccgtgtgattaccaccccacgtgatctggctacctatgttcattacgatgcactgtacgaggcatacctgaacgcttgtctgatcctgctgggtatgggtgctccgttcgatcctggtatcccgtttcagaaaccggatgttgaagataaacaacagggcttcgcacatttcggtggtccgcaaatcctgacgctggtatgtgaagctgcgactcgtggtctgaaagcggtgcgtttccagaaatttaatgtccaccgtcgtctgcgtccagaagcactgggcggtctggtggatcgttacaaacatggcaaaggtgccggcgatgaactgaaacctgtagcggcgctggttgaagcgctggaaaacgtaggcctgctgtctaaagttgtagcgcacaaccagctgcagaatcagaacctggatcgtagcggtgacccttcctctgcaggtgacaattatttcctgccaatggcttttccggaaggttctccaatgcacccgtcctacggtgcgggtcatgctaccgtcgcaggtgcttgcgtcacgatgctgaaagcattcttcgaccacggctggcagctgaatctgggcatggctaacggtaaatacatttcctacgaaccgaatcaggacggtagctccctgcagcaggttctgctggattgtccgctgacggtcgaaggtgagctgaacaaaatcgctgctaacatctccattggccgtgactgggctggtgtgcactacttcaccgattatatcgagtccctgcgtctgggcgaaaaaatcgcaattggcattctggaagaacagaaactgacgtatggcgaaaactttaccatgaccgtaccactgtatgatggcggttccatccaaatttaa

SEQ ID NO: 18 - DNA, 1920 bp, Artificial Sequence
  Open reading frame encoding Acaryochloris marina vanadium-dependent haloperoxidase codon optimised for expression in S. cerevisiae
atgaacactaggagacaacaagcccaaaacatcagaaacaatgccgctgaattggctgctaatagacctcacccacaacatataaataacaaagaggaatacgaatataggagaccaaaaaaagacggcaacgagccttcacacattgctaatttcaccaaggggttgcctcacgacgaacatacaggcttattactaaattccgccgactatgaccagttcgtcttaggtattcaatctggagacacgacggattttgctaggacgccattaggtcctgccgaattaccaaaagtacatggttgtttgtcaaaacaaaagattgactgtgatgatgaccacaggtctggattctggaagtcacaaatagctcaaggcgctgcgggtggtgatggagccaaacttagggcttgggaaagtgctggtgctggtttagtttttgacttagagggtccagatgcccaggcagtaacaatgccccctgctccgagattagaaagtccagagcttacttcagaaatagcagaagtgtattcacaagcattgctcagagacattcatttctctcagttacgtgaccctggcttgggagatcaagtaaatgcttgtgattcttgcccaacccaactttctatttatgaagcaatagatattctaaatactgtccagattgaaggccaaaattggttttcggccaattgttgtgatctcacagatgacgagcaggctcgtcagagaccactcgtaactcgacaaaatatatttcgtggcatcgcacctggagatgatgtcggtccctatctatcccagtttctgctgattggtaacaatgcgctgggaggtggtgtatttgggcaagaagccggccatatcggatatggtgctataagaattgatcagagagtcagaaaggctaccccctgcaaagactttatgactaacttcgaaacttggttggatgtgcaaaatggtgcagatttgaggggtctagaaacctacgttgacgctgatccgggaaaatgtagggaatttccagcgtatagagtcatcactacacctcgggacttagcaacctatgttcactacgatgcactatatgaggcatatttaaacgcttgcttgattttgctgggtatgggtgctccatttgaccccggtataccgttccaaaaaccagatgtggaagataaacagcaagggtttgcccattttggaggaccgcagattttgacactagtttgtgaggccgcaacaagagggttaaaagcagttagattccaaaagttcaacgttcatcgtcgtttgcgcccagaagctctaggtggtttagttgatagatataagcatggtaaaggtgcgggtgatgaacttaaacccgttgcagccttggtagaagcgctagagaatgttggtttgttgtcaaaggttgtagcccataatcaattgcaaaaccagaatcttgaccgcagtggcgatccatccagcgcaggggataattacttcctaccaatggccttcccagaaggttctcccatgcaccctagttatggtgcaggacatgcaactgtggctggagcttgtgtcactatgcttaaagcattttttgatcatggttggcaactgaatttgggtatggcgaatgggaaatacatatcctacgaacctaatcaagatgggagctcgcttcaacaagttcttttagattgccctctgacagtggaaggtgagttgaacaagattgcagctaacatctctattggtagagattgggctggcgtgcattactttacggattacatcgaatcgttacgattaggcgaaaagattgcgatcggcatattagaagagcaaaagctgacctacggagagaactttacaatgactgttccattgtacgatggaggcagcattcaaatctaa

SEQ ID NO: 19 - DNA, 1920 bp, Artificial Sequence
  Open reading frame encoding Acaryochloris marina vanadium-dependent haloperoxidase codon optimised for expression in P. pastoris
atgaatactaggagacaacaagcccaaaacatcaggaacaatgccgctgagctggctgctaaccgacctcacccacagcatataaacaacaaagaggaatacgaatatcgccgccctaaaaaagacggcaacgagccaagccacattgctaattttaccaaggggttgcctcacgacgaacatacaggcttattactcaactccgccgactatgaccagttcgtcttaggcattcagtctggagacacgacggattttgctagaacgccattgggtcctgccgaattaccaaaagttcatggttgtttgtcaaagcaaaagattgactgtgatgatgaccaccgttcaggattctggaagtcacagattgctcaaggagctgccggtggtgacggagccaaacttagggcttgggaaagtgcgggggctggtttagtttttgacttagaaggtcccgacgcccaggcagtaacaatgccacctgctccgagattagaaagtccagagcttacttcagaaatcgcagaagtatactcacaagcattgttgagagacattcatttctctcaattgcgagatcctggcttgggagatcaagtgaatgcctgtgattcttgcccaactcaactttctatttacgaagctatagatattctaaatactgtccagattgaaggccaaaattggttctctgccaattgttgcgatttgacagatgatgagcaggctcgtcagagaccattggtaactagacaaaacatatttcgtggaatcgctcctggagacgatgtcggtccctatctatcccagtttctgctgattggtaataatgcactgggaggtggggtatttgggcaagaggccggccatatcggatatggtgctatacgaattgatcagagagtcagaaaggctaccccctgcaaagacttcatgactaacttcgagacttggcttgatgtgcaaaacggtgcagatttgagaggtctagaaacctacgttgacgctgatccgggaaaatgtcgtgaatttccagcctatagagtcatcactacacctagagacctagctacctacgttcactacgatgcactatatgaggcatatttaaacgcttgcttgattttgctgggtatgggagcacctttcgacccaggtattccatttcagaaaccagatgtggaagataaacagcaaggatttgcccactttggaggaccgcaaatcttgacactagtttgtgaggccgcaacaagagggttaaaggcagttcgtttccaaaagttcaatgttcatagaagattgagacctgaagctctaggtggtctggttgataggtataagcacggtaagggtgctggtgacgagcttaaacccgttgcagccttggttgaagcgctggagaacgttggtctcttgagcaaagttgtagcccataatcagctccaaaaccagaaccttgaccggagtggtgatccttcctctgcaggtgacaattacttcctgccaatggccttcccagagggatctcccatgcacccatcgtatggtgctggacatgctactgtggctggagcttgtgtcactatgcttaaagcattctttgatcatggttggcaactgaacttgggtatggctaatggtaagtacatatcctacgaacctaatcaagatggatcgagtcttcaacaagtccttttagattgtcctttgacagtggaaggtgagttgaataagattgcagctaacatctctattggacgggattgggctggagtgcattactttaccgattacatcgaatcccttaggttgggcgaaaagattgcgatcggcattctcgaggagcaaaagctgacctacggagagaactttactatgaccgttcccctctatgatgggggctcgatacaaatctaa

SEQ ID NO: 20 - DNA, 21 bp, Artificial Sequence
  Primer specific for the 35S-AtVHPO transgene
atgaccgacacacagaatccc

SEQ ID NO: 21 - DNA, 22 bp, Artificial Sequence
  Primer specific for the 35S-AtVHPO transgene
ctagatacggatggtcgaaccg

SEQ ID NO: 22 - RNA, 27 nt, Artificial Sequence
  Nucleotide sequence of ePST1 tag for localisation to the peroxisome
uugggaagagguaggcgcuccaaacuu

SEQ ID NO: 23 - RNA, 21 nt, Artificial Sequence
  Primer sequence used for CTT1 deletion
ugccaaguacauagaauccac

SEQ ID NO: 24 - RNA, 27 nt, Artificial Sequence
  Primer sequence used for CTT1 deletion
agacaagagaaggauuuuuuuaauaag

SEQ ID NO: 25 - RNA, 85 nt, Artificial Sequence
  Primer sequence used for CTT1 deletion
cuuauuaaaaaaauccuucucuugucuuguacaagcacuaauauuucauacuuaucguacuucauacugauucgguaaucuccga

SEQ ID NO: 26 - RNA, 64 nt, Artificial Sequence
  Primer sequence used for CTT1 deletion
uacgaccaauuuguaauaacuagugauguagagaauaaaccuuccuuuuucaauggguaauaac

SEQ ID NO: 27 - RNA, 22 nt, Artificial Sequence
  Primer sequence for CTA1 deletion
gaacauccaaauucggaacuac

SEQ ID NO: 28 - RNA, 25 nt, Artificial Sequence
  Primer sequence for CTA1 deletion
cuaggguuccaaauuuauuuguaag

SEQ ID NO: 29 - RNA, 85 nt, Artificial Sequence
  Primer sequence for CTA1 deletion
cuuacaaauaaauuuggaacccuagguuaaaaaaauuaucauuucacacauaggaaagcucgucgugauucgguaaucuccgaac

SEQ ID NO: 30 - RNA, 26 nt, Artificial Sequence
  Primer sequence for CTA1 deletion
uuucuaacacgagaauaugagcauca

SEQ ID NO: 31 - RNA, 32 nt, Artificial Sequence
  Primer sequence for site directed mutagenesis
cuaccucuucccaagauacggauggucgaacc

SEQ ID NO: 32 - RNA, 31 nt, Artificial Sequence
  Primer sequence for site directed mutagenesis
gcgcuccaaacuuuagucuagagggccgcau

SEQ ID NO: 33 - RNA, 40 nt, Artificial Sequence
  Primer sequence for pRS-ePST1VHPO assembly
gcucgaaggcuuuaauuugcgaaccguaaaaaggccgcgu

SEQ ID NO: 34 - RNA, 41 nt, Artificial Sequence
  Primer sequence for pRS-ePST1VHPO assembly
uauauguaaguauacggcccgcccugauagacgguuuuucg

SEQ ID NO: 35 - RNA, 40 nt, Artificial Sequence
  Primer sequence for pRS-ePST1VHPO assembly
gaaaaaccgucuaucagggcgggccguauacuuacauaua

SEQ ID NO: 36 - RNA, 44 nt, Artificial Sequence
  Primer sequence for pRS-ePST1VHPO assembly
cuccuaguguucauaagcuucauguuuaguuaauuauaguucgu

SEQ ID NO: 37 - RNA, 40 nt, Artificial Sequence
  Primer sequence for pRS-ePST1VHPO
acuauaauuaacuaaacaugaagcuuaugaacacuaggag

SEQ ID NO: 38 - RNA, 40 nt, Artificial Sequence
  Primer sequence for pRS-ePST1VHPO assembly
acgcggccuuuuuacgguucgcaaauuaaagccuucgagc

SEQ ID NO: 39 - RNA, 23 nt, Artificial Sequence
  Primer sequence for pRS-ePST1VHPO sequencing
ggaacgaaaacucacguuaaggg

SEQ ID NO: 40 - RNA, 24 nt, Artificial Sequence
  Primer sequence for pRS-ePST1VHPO sequencing
gagcaaaaacaggaaggcaaaaug

SEQ ID NO: 41 - RNA, 18 nt, Artificial Sequence
  Primer sequence for pRS-ePST1VHPO sequencing
ugucggggcuggcuuaac

SEQ ID NO: 42 - RNA, 20 nt, Artificial Sequence
  Primer sequence for pRS-ePST1VHPO sequencing
auuaugacacccgguguggg

SEQ ID NO: 43 - RNA, 15 nt, Artificial Sequence
  Primer sequence for pRS-ePST1VHPO sequencing
cgcuuccccugccgg

SEQ ID NO: 44 - RNA, 22 nt, Artificial Sequence
  Primer sequence for pRS-ePST1VHPO sequencing
uggugauggagccaaacuuagg

SEQ ID NO: 45 - RNA, 15 nt, Artificial Sequence
  Primer sequence for pRS-ePST1VHPO sequencing
ucacgcccucccccc

SEQ ID NO: 46 - RNA, 19 nt, Artificial Sequence
  Primer sequence for pRS-ePST1VHPO sequencing
ggaccuagacuucagguug

SEQ ID NO: 47 - DNA, 3548 bp, Artificial Sequence
  Nucleotide sequence encoding CTA1-URA3
agaacatccaaattcggaactactaagaagatatgggtatgttgaatgggacggttcgaagtatgattttggagaagtgttacttgaaaatattgtcgaggcgttaaaagagacttttgagacgaatactgaatttttggacaggtgtattgatatcttacgcaataacgccaatattcaagaattcttagaaggtgaagaaatagtactagattcatatgattgttataataatggtgaattgttgcctcaactaatacttttggtccaaatcttgacaattctttgccaaattccaggtttatgcaaactggacataaaagcaatggaaaggcaagtggagagaattgtaaagaagtgtttacaattgatagaaggtgcccgcgccactacaaactgtagtgccacatggaaacgttgtattatgaagcgtctagccgattaccccataaaaaagtgcgtttctatcgaaaaaccttcgaaaggaaactcattaacaagggaagaactaagagatgttatggctcggagagttttgaaaagcgaaatagattcgctgcaagtttgtgaagaaaccatcgacaagaattacaaggttattcctgatgaaaagctgctaactaatattttaaagagaaagttgacagaggaagaaaaaagctctgtcaaacgtccttgcgtgaagaagtgagcggttgttctaaccactatttaaagccgcaattagtaatgcaaaaagttggccggaattagccgcgcaagttggtggggtcccttaatccgaaaaaggacggctttaacaaatataaactccgaaaatccccacagtgacagaattggagaaacaaccagttttgatatcgccatacatataaagagatgtagaaagcattcttcactgtaatgtccaaatcgtacatttgaatttcttgtaggtttatttaaaaggtaagttaaataaatataatagtacttacaaataaatttggaaccctagaagatgtcgaaattgggacaagaaaaaaatgaagtaaattactctgatgtaagagaggatagagttgtgacaaactccactggtaatccaatcaatgaaccatttgtcacccaacgtattggggaacatggccctttgcttttgcaagattataacttaattgattctttggctcatttcaacagggaaaatattcctcaaaggaatccacatgctcatggttctggtgccttcggctattttgaagtaaccgatgacattactgatatctgcgggtctgctatgtttagtaaaattgggaaaagaacgaaatgtctaacaagattttcgactgtgggtggtgataaaggtagtgccgacacggttcgtgatccaagggggtttgccaccaaattctacactgaagaaggtaatttagattgggtctacaataatacaccggtattctttatcagagacccttccaagttccctcactttatccacacacagaagagaaacccacaaaccaacctaagggatgctgacatgttttgggatttcctcaccactcctgaaaatcaggtggccattcatcaagtaatgatccttttttcagaccgtggtacccctgccaactaccgtagtatgcatggttattctggtcatacctataaatggtccaataaaaacggagattggcattatgtgcaagttcatatcaaaaccgatcaaggaataaagaatttgaccatagaagaggctaccaaaattgcgggatccaatccagattactgccagcaggatttatttgaggctattcagaatggaaactatccttcctggacagtttatattcaaacaatgaccgaacgcgatgccaaaaaattaccattttcagtctttgatttgactaaagtatggcctcaggggcaattccctttacggcgtgtgggtaagattgttttgaacgagaatccactgaacttcttcgcacaggtggaacaagctgccttcgcccccagtaccacggttccttaccaagaagcaagcgctgatccagtattacaggcccgtttgttttcatatgcggatgctcatagatacaggctaggtcctaacttccatcaaatacccgtaaactgtccatatgcatctaaatttttcaatcccgctatcagagatggaccgatgaatgttaacggcaacttcggctcagaacctacatatttggccaacgataaatcgtacacgtatatccaacaggacagacccattcaacaacaccaagaggtatggaatgggccagctatcccttatcattgggcaacatccccaggtgatgtagatttcgtgcaagcaagaaatctctaccgcgttttgggtaaacaacctggacagcaaaagaacttggcatataacatcggcattcatgtagaaggcgcctgtcctcaaatacagcagcgcgtttatgatatgtttgctcgtgttgataagggactatctgaggcaattaaaaaagtagctgaggcaaaacatgcttctgagctttcgagtaactccaaattttgaaacgctcaagtaacaaatgagtggcgttgtttccacgacaattatttatgatagtgtgtatttttaacacattttatttattacaatttatgtattttgttatgaattatttatttatacgactaataggtgatgctcatattctcgtgttagaaagttaaaaaaattatcatttcacacataggaaagctcgtcgcgccggggaaaaagctgaggaatctctattattaggggtaaagttcaacacattcagtatgagataagtgtgtcttcaagagagatgcagcactgagtagggaaccaagaaacgatgtctgaattactagatagctttgagacagagtttgcgaaattttataccgacagcaatctggaagagacaaaccttcaaaaatgtcttgatcatactcatgaattcaagagtcaattaaagaagttgaaagcgcacttaaacaagcatattcaggaatcaaaaccagaagtctataataagctgagtgataaggagaaacaaaagtttaagagaaaaagagaactaattattgagaagctaagcaagtcacagaggcaatgggaccattcagtaaagaaacaaataaagtacgtttctcagcagtcgaatcgcttcaacaaaagcacgctgaacaaattaaaagaatttgatatagatagcgtatatgtcaataaattaccgaaagagacaatggaaaatgttaatgaagccatcggttaccatattttgaggtatagtatagacaacatgccattaggaaataaaaatgaggctttccagtatctaaaagatgtctacggaattactaataaagaatctactgaatttattgagatgggccaaatcgtacatgatcttaaaaagggtgacacagaaagctgtctgaaatggtgctccaatgaaatggaaagcttatcatctaaccatacggctttatcgtccctaaaatttgatttatacactttgtcagcaatgcagatagtgaag

SEQ ID NO: 48 - DNA, 2098 bp, Artificial Sequence
  Nucleotide sequence encoding CTT1-URA3
tgccaagtacatagaatccacagtgcataggtttatgagatgcttggaagatgtacgatcgcctgcactatattagtatattttttcaggctttacaaaaccagaaagaaattaccgactgtaatacttaatttccatgattttaatcgtatggtccgtgaggaaagaggaattttaggtaaaaaaaaacctttgtctatcaaaacataaaagaaaagaaaaaaattaaattgaataagtcagctttttagcatgaccacagtaataatagtaatacgatatcagcatgagctgctaaacattaagaatttttgaattgtgttcgctggtaagagaaaatagggtagcattagttgcgagatggaaaattaaaatatgcgtcataaggtattttcgacgttgaagattaggggttgcaagtccgagaactaaactgccttcacgacgtcaaacttattgtatggtgacaataaaacgaattcaagaagccagagatgatgaggaaccgatgacgaggcacatggggatagaacctccgttatctccattccgcgttgagcagctctcctgcgtgctttcacagagaccctaagccaagaccgcggacggccctctttttcaaggggatcaccggtaaggggccaagcggtgaaattgcgtattgtttcctcctatcaatgatgagtacgtcgccgatccagaagggttattattgtcattgccagtactattcgtatgctgaaaaagtatagttcacttcaccctctggctgcaggctagcctagccgataatgcccccgactccctcaacaggtagaatttgtaaggattcgacgtagcctggacacattgtgcatttatcgtatcccctactgctacacgtataagaagtcgttgctttacctctattccagaactcaatcttgtcgttacttgcccttattaaaaaaatccttctcttgtcttgtacaagcactaatatttcatacttatcgtacttcatactgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccattgaaaaaggaaggtttattctctacatcactagttattacaaattggtcgta

SEQ ID NO: 49 - DNA, 5880 bp, Artificial Sequence
  Nucleotide sequence encoding pACYCDuet-1-VBPO
ggggaattgtgagcggataacaattcccctgtagaaataattttgtttaactttaataaggagatataccatgggcagcagccatcaccatcatcaccacagccaggatccgaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgaacactcgtcgtcagcaggcacagaacatccgtaataatgcagccgaactggctgcgaatcgtccacacccgcagcatatcaacaacaaagaggaatacgaatatcgtcgtccgaaaaaagacggcaatgaaccgagccacatcgctaacttcactaagggcctgccgcacgatgaacatactggtctgctgctgaactctgcggactacgaccaattcgttctgggtattcagagcggtgacactacggattttgcacgtacgccactgggtccggctgaactgcctaaagtacacggttgtctgtctaaacaaaaaattgattgcgatgatgatcatcgttccggcttctggaaatctcagatcgcgcaaggtgcagcaggtggtgatggtgctaaactgcgtgcttgggagagcgcaggtgctggtctggtcttcgacctggaaggtccagacgcacaggcagtaaccatgccaccggctccacgtctggaatcccctgaactgactagcgagatcgcagaagtgtactcccaggctctgctgcgtgatattcatttctcccagctgcgtgatccgggtctgggtgatcaggtgaacgcttgcgactcctgcccgactcagctgagcatctatgaagctatcgacattctgaacaccgtacagatcgaaggtcagaactggttcagcgctaattgttgcgatctgaccgatgatgaacaggcacgtcagcgtccgctggtaacccgtcagaacattttccgtggtatcgcgccaggcgatgatgttggtccgtacctgtctcagttcctgctgatcggtaacaacgcactgggcggtggcgtattcggtcaggaagctggtcatatcggttacggtgcaattcgtatcgatcaacgcgtccgtaaagcaaccccgtgcaaggactttatgaccaacttcgaaacttggctggacgttcagaacggcgctgacctgcgtggcctggaaacgtatgtggatgctgatccgggcaaatgccgtgagttcccggcataccgtgtgattaccaccccacgtgatctggctacctatgttcattacgatgcactgtacgaggcatacctgaacgcttgtctgatcctgctgggtatgggtgctccgttcgatcctggtatcccgtttcagaaaccggatgttgaagataaacaacagggcttcgcacatttcggtggtccgcaaatcctgacgctggtatgtgaagctgcgactcgtggtctgaaagcggtgcgtttccagaaatttaatgtccaccgtcgtctgcgtccagaagcactgggcggtctggtggatcgttacaaacatggcaaaggtgccggcgatgaactgaaacctgtagcggcgctggttgaagcgctggaaaacgtaggcctgctgtctaaagttgtagcgcacaaccagctgcagaatcagaacctggatcgtagcggtgacccttcctctgcaggtgacaattatttcctgccaatggcttttccggaaggttctccaatgcacccgtcctacggtgcgggtcatgctaccgtcgcaggtgcttgcgtcacgatgctgaaagcattcttcgaccacggctggcagctgaatctgggcatggctaacggtaaatacatttcctacgaaccgaatcaggacggtagctccctgcagcaggttctgctggattgtccgctgacggtcgaaggtgagctgaacaaaatcgctgctaacatctccattggccgtgactgggctggtgtgcactacttcaccgattatatcgagtccctgcgtctgggcgaaaaaatcgcaattggcattctggaagaacagaaactgacgtatggcgaaaactttaccatgaccgtaccactgtatgatggcggttccatccaaatttaaggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 50 - DNA, 11883 bp, Artificial Sequence
  Nucleotide sequence encoding pCRISPYL-34265gRNA1
cacatttccccgaaaagtgccacctgacgtccccagttgcaaaagttgacacaactctagatctgcttccaaatatagaatcataacaagggttagggtgtgattatataatattggtcttaattgatgtgctagggctttaaaagttggttaaaataacgctctaatgcctttttaatatattgtctttttcaaaatctcaaatcggacacttcttcgtgtatgagactccattttttggctccgtcacgtgatatggggtcggcgcaggttgacgttattatcagctatagtggtgtaaacaaagttttttactagctgtaatggcattttgtcggagtggtaaatcgccttcttgttgtgcgttcgagttctggactctgcactgggctactttgaaaaatacctctaatgcgccgatggtttagtggtaaaatccatcgttgccatcgatgggcccccggttcgattccgggtcggcgcaggttgaccgggtcggcgcaggttgacgtggcgtagagttctttggacagttttagagctagaaatagcggttttagagctagaaatagcaagttaaaataaggctagtccgttatcaacttgaaaaagtggcaccgagtcggtgcttttttttacgtctaagaaaccattattatcatgacattaacctataaaaataggcgtatcacgaggcccagatcctctagagtcgaagcggccgctatgtctgataaaaggatgtaacataggcaagctgctcgtgagtgttgagtacgaaccttagatccaaatcacccgcacccacggatatacttgcttgaatatacagtagtatgcggccgcttcgacaccatatcatataaaactaacaatgcattgaattaatacgaagactacccgttgctatctccacaccgttatctccacggtccaaaggctgctcaatgtgctgcatacgtaacgtggggtgcaaccttgagcacatagtactttccgaaaaccggcgataattaagtgtgcactccaacttttcacactgagcataaaatgtggagaagaaatcacactaaaaagtcaggtagactggaaaatgcgccatgaaataatatctcttgctacagtaatgcccagcatcgaggggtattgtgtcaccaacactatagtggcagctgaagcgctcgtgattgtagtatgagtctttattggtgatgggaagagttcactcaatattctcgttactgccaaaacaccacggtaatcggccagacaccatggatgtagatcaccaagcctgtgaatgttattcgagctaaaatgcacatggttggtgaaaggagtagttgctgtcgaattccgtcgtcgcctgagtcatcatttatttaccagttggccacaaacccttgacgatctcgtatgtcccctccgacatactcccggccggctggggtacgttcgatagcgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgtttgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtctcctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacaccgagacctactccgttcctgaggttgagcgaattgcccgaatggccgccttcctggcccttcagcacaacccccctcttcccgtgtggtctcttgacaaggccaacgtgctggcctcctctcgactttggcgaaagactgtcactcgagtcctcaaggacgaattcccccagctcgagctcaaccaccagctgatcgactcggccgccatgatcctcatcaagcagccctccaagatgaatggtatcatcatcaccaccaacatgtttggcgatatcatctccgacgaggcctccgtcatccccggttctctgggtctgctgccctccgcctctctggcttctctgcccgacaccaacgaggcgttcggtctgtacgagccctgtcacggatctgcccccgatctcggcaagcagaaggtcaaccccattgccaccattctgtctgccgccatgatgctcaagttctctcttaacatgaagcccgccggtgacgctgttgaggctgccgtcaaggagtccgtcgaggctggtatcactaccgccgatatcggaggctcttcctccacctccgaggtcggagactttgttgccaacaaggtcaaggagctgctcaagaaggagtaagtcgtttctacgacgcattgatggaaggagcaaactgacgcgcctgcgggttggtctaccggcagggtccgctagtgtataagactctataaaaagggcccagccctgctaatgaaatgatgatttataatttaccggtgtagcaaccttgactagaagaagcagattgggtgtgtttgtagtggaggacagtggtacgttttggaaacagtcttcttgaaagtgtcttgtctacagtatattcactcataacctcaatagccaagggtgtagtcggtttattaaaggaagggagttgtggctgatgtggatagatatctttaaagctggcgactgcacccaacgagtgtggtggtagcttgttactgtatattcggtaagatatattttgtggggtttttagtggtgtttggtaggttagtgtctggtatatgagttgtaggcatgacaatttggaaaggggtggactttgggaatattgtgggatttcaataccttagtttgtacagggtaattgttacaaatgatacaaagaactgtatttcttttcatttgttttaattggttgtatatcaagtccgttagacgagctcagtgccatggcttttggcactgtatttcatttttagaggtacactacatccagtgaggtatggtaaggttgagggcataatgaaggcaccttgtactgacagtcacagacctctcaccgagaattttatgagatatactcgggttcattttaggctccgattcgattcaaattattactgtcgaaatcggttgagcatccgttgatttccgaacagatctggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgcttttcgtagataatggaatacaaatggatatccagagtatacacatggatagtatacactgacacgacaattctgtatctctttatgttaactactgtgaggcgttaaatagagcttgatatataaaatgttacatttcacagtctgaacttttgcagattacctaatttggtaagatattaattatgaactgaaagttgatggcatccctaaatttgatgaaagggggatcccccgggttcgaaggtaccaaggaagcatgcggtacccgaattcctgaggtgtctcacaagtgccgtgcagtcccgcccccacttgcttctctttgtgtgtagtgtacgtacattatcgagaccgttgttcccgcccacctcgatccggtctagactgaggtgtctcacaagtgccgtgcagtcccgcccccacttgcttctctttgtgtgtagtgtacgtacattatcgagaccgttgttcccgcccacctcgatccggggatccctgaggtgtctcacaagtgccgtgcagtcccgcccccacttgcttctctttgtgtgtagtgtacgtacattatcgagaccgttgttcccgcccacctcgatccgggtcgacctgaggtgtctcacaagtgccgtgcagtcccgcccccacttgcttctctttgtgtgtagtgtacgtacattatcgagaccgttgttcccgcccacctcgatccgggagctcctgaggtgtctcacaagtgccgtgcagtcccgcccccacttgcttctctttgtgtgtagtgtacgtacattatcgagaccgttgttcccgcccacctcgatccggtctagactgaggtgtctcacaagtgccgtgcagtcccgcccccacttgcttctctttgtgtgtagtgtacgtacattatcgagaccgttgttcccgcccacctcgatccggggatccctgaggtgtctcacaagtgccgtgcagtcccgcccccacttgcttctctttgtgtgtagtgtacgtacattatcgagaccgttgttcccgcccacctcgatccgggtcgacctgaggtgtctcacaagtgccgtgcagtcccgcccccacttgcttctctttgtgtgtagtgtacgtacattatcgagaccgttgttcccgcccacctcgatccgggcatgcctgcagaagcttttgtggttgggactttagccaagggtataaaagaccaccgtccccgaattacctttcctcttcttttctctctctccttgtcaactcacacccgaaatcgttaagcatttccttctgagtataagaatcattcaaaggcgcgccacaatggataagaaatactccattggcctggacatcggaaccaactccgtgggttgggccgtgatcaccgatgagtacaaggtgccctctaagaaattcaaggtcctgggcaacaccgaccgacactccatcaagaagaacctgatcggcgctctgctcttcgactctggcgagaccgctgaggccacccgactgaagcgaaccgctcgaagacgatacacccgaagaaagaaccgaatctgttacctgcaggagatcttctctaacgagatggccaaggtggacgactctttcttccaccgactggaggagtctttcctggtggaggaggacaagaagcacgagcgacaccccatcttcggcaacatcgtggacgaggtggcctaccacgagaagtaccccaccatctaccacctgcgaaagaagctggtggactctaccgacaaggccgacctgcgactgatctacctggccctggcccacatgatcaagttccgaggccacttcctgatcgagggcgacctgaaccccgacaactctgacgtggacaagctgttcatccagctggtgcagacctacaaccagctcttcgaagagaaccccattaacgcttctggcgtggatgctaaggccatcctgtctgcccgactgtctaagtctcgacgactcgagaacctgattgctcagctccccggagagaagaagaacggtctgttcggaaacctgattgctctgtccctgggtctcacccctaacttcaagtccaacttcgatctggctgaggacgctaagctgcagctgtctaaggacacctacgacgatgacctggataacctgctcgcccagattggcgaccagtacgccgacctgttcctggccgccaagaacctgtctgacgccatcctgctgtctgacatcctgcgagtgaacaccgagatcaccaaggcccccctgtctgcctccatgattaagcgatacgatgagcaccaccaggatctgaccctcctcaaggctctggtccgacagcagctgcccgagaagtacaaggagattttcttcgaccagtctaagaacggctacgccggctacatcgacggcggcgcctctcaggaggagttctacaagttcattaagcccatcctggagaagatggacggaaccgaggaactgctcgtgaagctgaaccgagaggacctcctgcgaaagcagcgaaccttcgacaacggctctatcccccaccagatccacctgggcgagctgcacgccatcctgcgacgacaggaggacttctaccccttcctgaaggacaaccgagagaagatcgagaagatcctgaccttccgaatcccctactacgtgggacccctggcccgaggaaactctcgattcgcttggatgacccgaaagtctgaggagaccattaccccctggaacttcgaggaggtggtggataagggcgcctctgctcagtctttcatcgagcgaatgaccaacttcgacaagaacctccccaacgagaaggtcctgcccaagcactctctgctctacgagtacttcaccgtctacaacgagctcaccaaggtcaagtacgtgaccgagggaatgcgaaagcccgctttcctgtctggagagcagaagaaggctattgtggatctgctcttcaagactaaccgaaaggtcaccgtcaagcagctgaaggaggattacttcaagaagattgagtgtttcgattctgtcgagatctccggcgtcgaggaccgattcaacgcctctctgggtacctaccacgacctgctgaagattatcaaggacaaggatttcctggataacgaggagaacgaggatattctcgaggacattgtcctgaccctcaccctgttcgaggatcgagagatgattgaggagcgactcaagacctacgctcacctgttcgacgacaaggtgatgaagcagctgaagcgacgacgatacaccggctggggccgactgtctcgaaagctgatcaacggcatccgagacaagcagtctggcaagaccatcctggacttcctgaagtctgacggcttcgccaaccgaaacttcatgcagctgatccacgacgactctctgaccttcaaggaggacatccagaaggcccaggtgtctggccagggcgactctctgcacgagcacatcgccaacctggccggctctcccgccattaagaaaggtatcctgcagaccgtcaaggtggtcgatgagctcgtcaaggtgatgggccgacacaagcccgagaacattgtcattgagatggctcgagagaaccagactactcagaagggtcagaaaaactcccgagagcgaatgaagcgaattgaggaaggtattaaggagctgggatcccagattctcaaggagcatcccgtggagaacactcagctccagaacgagaagctgtacctgtactatctgcagaacggtcgagacatgtacgtcgaccaggagctggatatcaaccgactctccgactacgatgtggaccacattgtgccccagtccttcctgaaggacgattctatcgataacaaggtgctgacccgatccgacaagaaccgaggcaagtctgacaacgtgccctccgaggaggtggtcaagaagatgaagaactactggcgacagctgctgaacgccaagctgattacccagcgaaagttcgacaacctgaccaaggccgagcgaggcggcctgtctgagctggacaaggccggcttcatcaagcgacagctggtggagacccgacagatcaccaagcacgtggcccagatcctggactctcgaatgaacaccaagtacgacgagaacgacaagctgatccgagaggtgaaggtgatcaccctgaagtctaagctggtgtctgacttccgaaaggacttccagttctacaaggtgcgagagattaacaactaccaccacgcccacgatgcctacctgaacgctgtcgtgggcaccgccctcatcaagaagtatcccaagctggagtccgagttcgtctacggcgactacaaggtctacgatgtgcgaaaaatgattgccaagtccgagcaggagattggcaaggctaccgccaagtacttcttctactccaacattatgaacttcttcaagaccgagattaccctggctaacggcgagattcgaaagcgacccctcattgagaccaacggagagaccggtgagatcgtgtgggacaagggacgagacttcgccaccgtgcgaaaggtgctgtctatgccccaggtgaacatcgtgaagaagaccgaggtgcagaccggaggtttctctaaggagtccatcctgcccaagcgaaactctgacaagctgatcgcccgaaagaaggactgggaccccaagaagtacggaggtttcgactctcccaccgtggcttactctgtgctggtggtggccaaggtggagaagggcaagtctaagaagctgaagtctgtgaaggagctgctgggcattaccatcatggagcgatcttctttcgagaagaaccccattgacttcctggaggccaagggatacaaggaggtgaagaaagatctgattatcaagctccccaagtactctctgttcgagctggagaacggacgaaagcgaatgctggcctctgccggcgagctgcagaagggaaacgagctggccctgccctccaagtacgtcaacttcctgtacctcgcctcccattacgagaagctgaagggctctcccgaggataacgagcagaagcagctcttcgtggagcagcataagcactacctggacgagatcatcgagcagatctctgagttctctaagcgagtgatcctggctgacgccaacctggataaggtgctgtctgcttacaacaagcaccgagacaagcccattcgagagcaggctgagaacatcattcacctgttcaccctgaccaacctgggagcccccgctgccttcaagtacttcgacaccaccatcgaccgaaagcgatacacctctaccaaggaggtgctggacgccaccctgatccaccagtctatcaccggcctgtacgagacccgaatcgacctgtctcagctgggcggcgactctcgagccgaccccaagaagaagcgaaaggtgtaagctagcctcatgtaattagttatgtcacgcttacattcacgccctccctccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgca

SEQ ID NO: 51 - DNA, 11883 bp, Artificial Sequence
  Nucleotide sequence encoding pCRISPYL-34265gRNA2
cacatttccccgaaaagtgccacctgacgtccccagttgcaaaagttgacacaactctagatctgcttccaaatatagaatcataacaagggttagggtgtgattatataatattggtcttaattgatgtgctagggctttaaaagttggttaaaataacgctctaatgcctttttaatatattgtctttttcaaaatctcaaatcggacacttcttcgtgtatgagactccattttttggctccgtcacgtgatatggggtcggcgcaggttgacgttattatcagctatagtggtgtaaacaaagttttttactagctgtaatggcattttgtcggagtggtaaatcgccttcttgttgtgcgttcgagttctggactctgcactgggctactttgaaaaatacctctaatgcgccgatggtttagtggtaaaatccatcgttgccatcgatgggcccccggttcgattccgggtcggcgcaggttgaccgggtcggcgcaggttgacgtgtaaggaactccaaaggagtgttttagagctagaaatagcggttttagagctagaaatagcaagttaaaataaggctagtccgttatcaacttgaaaaagtggcaccgagtcggtgcttttttttacgtctaagaaaccattattatcatgacattaacctataaaaataggcgtatcacgaggcccagatcctctagagtcgaagcggccgctatgtctgataaaaggatgtaacataggcaagctgctcgtgagtgttgagtacgaaccttagatccaaatcacccgcacccacggatatacttgcttgaatatacagtagtatgcggccgcttcgacaccatatcatataaaactaacaatgcattgaattaatacgaagactacccgttgctatctccacaccgttatctccacggtccaaaggctgctcaatgtgctgcatacgtaacgtggggtgcaaccttgagcacatagtactttccgaaaaccggcgataattaagtgtgcactccaacttttcacactgagcataaaatgtggagaagaaatcacactaaaaagtcaggtagactggaaaatgcgccatgaaataatatctcttgctacagtaatgcccagcatcgaggggtattgtgtcaccaacactatagtggcagctgaagcgctcgtgattgtagtatgagtctttattggtgatgggaagagttcactcaatattctcgttactgccaaaacaccacggtaatcggccagacaccatggatgtagatcaccaagcctgtgaatgttattcgagctaaaatgcacatggttggtgaaaggagtagttgctgtcgaattccgtcgtcgcctgagtcatcatttatttaccagttggccacaaacccttgacgatctcgtatgtcccctccgacatactcccggccggctggggtacgttcgatagcgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgtttgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtctcctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacaccgagacctactccgttcctgaggttgagcgaattgcccgaatggccgccttcctggcccttcagcacaacccccctcttcccgtgtggtctcttgacaaggccaacgtgctggcctcctctcgactttggcgaaagactgtcactcgagtcctcaaggacgaattcccccagctcgagctcaaccaccagctgatcgactcggccgccatgatcctcatcaagcagccctccaagatgaatggtatcatcatcaccaccaacatgtttggcgatatcatctccgacgaggcctccgtcatccccggttctctgggtctgctgccctccgcctctctggcttctctgcccgacaccaacgaggcgttcggtctgtacgagccctgtcacggatctgcccccgatctcggcaagcagaaggtcaaccccattgccaccattctgtctgccgccatgatgctcaagttctctcttaacatgaagcccgccggtgacgctgttgaggctgccgtcaaggagtccgtcgaggctggtatcactaccgccgatatcggaggctcttcctccacctccgaggtcggagactttgttgccaacaaggtcaaggagctgctcaagaaggagtaagtcgtttctacgacgcattgatggaaggagcaaactgacgcgcctgcgggttggtctaccggcagggtccgctagtgtataagactctataaaaagggcccagccctgctaatgaaatgatgatttataatttaccggtgtagcaaccttgactagaagaagcagattgggtgtgtttgtagtggaggacagtggtacgttttggaaacagtcttcttgaaagtgtcttgtctacagtatattcactcataacctcaatagccaagggtgtagtcggtttattaaaggaagggagttgtggctgatgtggatagatatctttaaagctggcgactgcacccaacgagtgtggtggtagcttgttactgtatattcggtaagatatattttgtggggtttttagtggtgtttggtaggttagtgtctggtatatgagttgtaggcatgacaatttggaaaggggtggactttgggaatattgtgggatttcaataccttagtttgtacagggtaattgttacaaatgatacaaagaactgtatttcttttcatttgttttaattggttgtatatcaagtccgttagacgagctcagtgccatggcttttggcactgtatttcatttttagaggtacactacatccagtgaggtatggtaaggttgagggcataatgaaggcaccttgtactgacagtcacagacctctcaccgagaattttatgagatatactcgggttcattttaggctccgattcgattcaaattattactgtcgaaatcggttgagcatccgttgatttccgaacagatctggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgcttttcgtagataatggaatacaaatggatatccagagtatacacatggatagtatacactgacacgacaattctgtatctctttatgttaactactgtgaggcgttaaatagagcttgatatataaaatgttacatttcacagtctgaacttttgcagattacctaatttggtaagatattaattatgaactgaaagttgatggcatccctaaatttgatgaaagggggatcccccgggttcgaaggtaccaaggaagcatgcggtacccgaattcctgaggtgtctcacaagtgccgtgcagtcccgcccccacttgcttctctttgtgtgtagtgtacgtacattatcgagaccgttgttcccgcccacctcgatccggtctagactgaggtgtctcacaagtgccgtgcagtcccgcccccacttgcttctctttgtgtgtagtgtacgtacattatcgagaccgttgttcccgcccacctcgatccggggatccctgaggtgtctcacaagtgccgtgcagtcccgcccccacttgcttctctttgtgtgtagtgtacgtacattatcgagaccgttgttcccgcccacctcgatccgggtcgacctgaggtgtctcacaagtgccgtgcagtcccgcccccacttgcttctctttgtgtgtagtgtacgtacattatcgagaccgttgttcccgcccacctcgatccgggagctcctgaggtgtctcacaagtgccgtgcagtcccgcccccacttgcttctctttgtgtgtagtgtacgtacattatcgagaccgttgttcccgcccacctcgatccggtctagactgaggtgtctcacaagtgccgtgcagtcccgcccccacttgcttctctttgtgtgtagtgtacgtacattatcgagaccgttgttcccgcccacctcgatccggggatccctgaggtgtctcacaagtgccgtgcagtcccgcccccacttgcttctctttgtgtgtagtgtacgtacattatcgagaccgttgttcccgcccacctcgatccgggtcgacctgaggtgtctcacaagtgccgtgcagtcccgcccccacttgcttctctttgtgtgtagtgtacgtacattatcgagaccgttgttcccgcccacctcgatccgggcatgcctgcagaagcttttgtggttgggactttagccaagggtataaaagaccaccgtccccgaattacctttcctcttcttttctctctctccttgtcaactcacacccgaaatcgttaagcatttccttctgagtataagaatcattcaaaggcgcgccacaatggataagaaatactccattggcctggacatcggaaccaactccgtgggttgggccgtgatcaccgatgagtacaaggtgccctctaagaaattcaaggtcctgggcaacaccgaccgacactccatcaagaagaacctgatcggcgctctgctcttcgactctggcgagaccgctgaggccacccgactgaagcgaaccgctcgaagacgatacacccgaagaaagaaccgaatctgttacctgcaggagatcttctctaacgagatggccaaggtggacgactctttcttccaccgactggaggagtctttcctggtggaggaggacaagaagcacgagcgacaccccatcttcggcaacatcgtggacgaggtggcctaccacgagaagtaccccaccatctaccacctgcgaaagaagctggtggactctaccgacaaggccgacctgcgactgatctacctggccctggcccacatgatcaagttccgaggccacttcctgatcgagggcgacctgaaccccgacaactctgacgtggacaagctgttcatccagctggtgcagacctacaaccagctcttcgaagagaaccccattaacgcttctggcgtggatgctaaggccatcctgtctgcccgactgtctaagtctcgacgactcgagaacctgattgctcagctccccggagagaagaagaacggtctgttcggaaacctgattgctctgtccctgggtctcacccctaacttcaagtccaacttcgatctggctgaggacgctaagctgcagctgtctaaggacacctacgacgatgacctggataacctgctcgcccagattggcgaccagtacgccgacctgttcctggccgccaagaacctgtctgacgccatcctgctgtctgacatcctgcgagtgaacaccgagatcaccaaggcccccctgtctgcctccatgattaagcgatacgatgagcaccaccaggatctgaccctcctcaaggctctggtccgacagcagctgcccgagaagtacaaggagattttcttcgaccagtctaagaacggctacgccggctacatcgacggcggcgcctctcaggaggagttctacaagttcattaagcccatcctggagaagatggacggaaccgaggaactgctcgtgaagctgaaccgagaggacctcctgcgaaagcagcgaaccttcgacaacggctctatcccccaccagatccacctgggcgagctgcacgccatcctgcgacgacaggaggacttctaccccttcctgaaggacaaccgagagaagatcgagaagatcctgaccttccgaatcccctactacgtgggacccctggcccgaggaaactctcgattcgcttggatgacccgaaagtctgaggagaccattaccccctggaacttcgaggaggtggtggataagggcgcctctgctcagtctttcatcgagcgaatgaccaacttcgacaagaacctccccaacgagaaggtcctgcccaagcactctctgctctacgagtacttcaccgtctacaacgagctcaccaaggtcaagtacgtgaccgagggaatgcgaaagcccgctttcctgtctggagagcagaagaaggctattgtggatctgctcttcaagactaaccgaaaggtcaccgtcaagcagctgaaggaggattacttcaagaagattgagtgtttcgattctgtcgagatctccggcgtcgaggaccgattcaacgcctctctgggtacctaccacgacctgctgaagattatcaaggacaaggatttcctggataacgaggagaacgaggatattctcgaggacattgtcctgaccctcaccctgttcgaggatcgagagatgattgaggagcgactcaagacctacgctcacctgttcgacgacaaggtgatgaagcagctgaagcgacgacgatacaccggctggggccgactgtctcgaaagctgatcaacggcatccgagacaagcagtctggcaagaccatcctggacttcctgaagtctgacggcttcgccaaccgaaacttcatgcagctgatccacgacgactctctgaccttcaaggaggacatccagaaggcccaggtgtctggccagggcgactctctgcacgagcacatcgccaacctggccggctctcccgccattaagaaaggtatcctgcagaccgtcaaggtggtcgatgagctcgtcaaggtgatgggccgacacaagcccgagaacattgtcattgagatggctcgagagaaccagactactcagaagggtcagaaaaactcccgagagcgaatgaagcgaattgaggaaggtattaaggagctgggatcccagattctcaaggagcatcccgtggagaacactcagctccagaacgagaagctgtacctgtactatctgcagaacggtcgagacatgtacgtcgaccaggagctggatatcaaccgactctccgactacgatgtggaccacattgtgccccagtccttcctgaaggacgattctatcgataacaaggtgctgacccgatccgacaagaaccgaggcaagtctgacaacgtgccctccgaggaggtggtcaagaagatgaagaactactggcgacagctgctgaacgccaagctgattacccagcgaaagttcgacaacctgaccaaggccgagcgaggcggcctgtctgagctggacaaggccggcttcatcaagcgacagctggtggagacccgacagatcaccaagcacgtggcccagatcctggactctcgaatgaacaccaagtacgacgagaacgacaagctgatccgagaggtgaaggtgatcaccctgaagtctaagctggtgtctgacttccgaaaggacttccagttctacaaggtgcgagagattaacaactaccaccacgcccacgatgcctacctgaacgctgtcgtgggcaccgccctcatcaagaagtatcccaagctggagtccgagttcgtctacggcgactacaaggtctacgatgtgcgaaaaatgattgccaagtccgagcaggagattggcaaggctaccgccaagtacttcttctactccaacattatgaacttcttcaagaccgagattaccctggctaacggcgagattcgaaagcgacccctcattgagaccaacggagagaccggtgagatcgtgtgggacaagggacgagacttcgccaccgtgcgaaaggtgctgtctatgccccaggtgaacatcgtgaagaagaccgaggtgcagaccggaggtttctctaaggagtccatcctgcccaagcgaaactctgacaagctgatcgcccgaaagaaggactgggaccccaagaagtacggaggtttcgactctcccaccgtggcttactctgtgctggtggtggccaaggtggagaagggcaagtctaagaagctgaagtctgtgaaggagctgctgggcattaccatcatggagcgatcttctttcgagaagaaccccattgacttcctggaggccaagggatacaaggaggtgaagaaagatctgattatcaagctccccaagtactctctgttcgagctggagaacggacgaaagcgaatgctggcctctgccggcgagctgcagaagggaaacgagctggccctgccctccaagtacgtcaacttcctgtacctcgcctcccattacgagaagctgaagggctctcccgaggataacgagcagaagcagctcttcgtggagcagcataagcactacctggacgagatcatcgagcagatctctgagttctctaagcgagtgatcctggctgacgccaacctggataaggtgctgtctgcttacaacaagcaccgagacaagcccattcgagagcaggctgagaacatcattcacctgttcaccctgaccaacctgggagcccccgctgccttcaagtacttcgacaccaccatcgaccgaaagcgatacacctctaccaaggaggtgctggacgccaccctgatccaccagtctatcaccggcctgtacgagacccgaatcgacctgtctcagctgggcggcgactctcgagccgaccccaagaagaagcgaaaggtgtaagctagcctcatgtaattagttatgtcacgcttacattcacgccctccctccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgca

SEQ ID NO: 52 - DNA, 11883 bp, Artificial Sequence
  Nucleotide sequence encoding pCRISPYL-34749gRNA1
cacatttccccgaaaagtgccacctgacgtccccagttgcaaaagttgacacaactctagatctgcttccaaatatagaatcataacaagggttagggtgtgattatataatattggtcttaattgatgtgctagggctttaaaagttggttaaaataacgctctaatgcctttttaatatattgtctttttcaaaatctcaaatcggacacttcttcgtgtatgagactccattttttggctccgtcacgtgatatggggtcggcgcaggttgacgttattatcagctatagtggtgtaaacaaagttttttactagctgtaatggcattttgtcggagtggtaaatcgccttcttgttgtgcgttcgagttctggactctgcactgggctactttgaaaaatacctctaatgcgccgatggtttagtggtaaaatccatcgttgccatcgatgggcccccggttcgattccgggtcggcgcaggttgaccgggtcggcgcaggttgacgtgtgggggaaggggactccgagttttagagctagaaatagcggttttagagctagaaatagcaagttaaaataaggctagtccgttatcaacttgaaaaagtggcaccgagtcggtgcttttttttacgtctaagaaaccattattatcatgacattaacctataaaaataggcgtatcacgaggcccagatcctctagagtcgaagcggccgctatgtctgataaaaggatgtaacataggcaagctgctcgtgagtgttgagtacgaaccttagatccaaatcacccgcacccacggatatacttgcttgaatatacagtagtatgcggccgcttcgacaccatatcatataaaactaacaatgcattgaattaatacgaagactacccgttgctatctccacaccgttatctccacggtccaaaggctgctcaatgtgctgcatacgtaacgtggggtgcaaccttgagcacatagtactttccgaaaaccggcgataattaagtgtgcactccaacttttcacactgagcataaaatgtggagaagaaatcacactaaaaagtcaggtagactggaaaatgcgccatgaaataatatctcttgctacagtaatgcccagcatcgaggggtattgtgtcaccaacactatagtggcagctgaagcgctcgtgattgtagtatgagtctttattggtgatgggaagagttcactcaatattctcgttactgccaaaacaccacggtaatcggccagacaccatggatgtagatcaccaagcctgtgaatgttattcgagctaaaatgcacatggttggtgaaaggagtagttgctgtcgaattccgtcgtcgcctgagtcatcatttatttaccagttggccacaaacccttgacgatctcgtatgtcccctccgacatactcccggccggctggggtacgttcgatagcgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgtttgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtctcctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacaccgagacctactccgttcctgaggttgagcgaattgcccgaatggccgccttcctggcccttcagcacaacccccctcttcccgtgtggtctcttgacaaggccaacgtgctggcctcctctcgactttggcgaaagactgtcactcgagtcctcaaggacgaattcccccagctcgagctcaaccaccagctgatcgactcggccgccatgatcctcatcaagcagccctccaagatgaatggtatcatcatcaccaccaacatgtttggcgatatcatctccgacgaggcctccgtcatccccggttctctgggtctgctgccctccgcctctctggcttctctgcccgacaccaacgaggcgttcggtctgtacgagccctgtcacggatctgcccccgatctcggcaagcagaaggtcaaccccattgccaccattctgtctgccgccatgatgctcaagttctctcttaacatgaagcccgccggtgacgctgttgaggctgccgtcaaggagtccgtcgaggctggtatcactaccgccgatatcggaggctcttcctccacctccgaggtcggagactttgttgccaacaaggtcaaggagctgctcaagaaggagtaagtcgtttctacgacgcattgatggaaggagcaaactgacgcgcctgcgggttggtctaccggcagggtccgctagtgtataagactctataaaaagggcccagccctgctaatgaaatgatgatttataatttaccggtgtagcaaccttgactagaagaagcagattgggtgtgtttgtagtggaggacagtggtacgttttggaaacagtcttcttgaaagtgtcttgtctacagtatattcactcataacctcaatagccaagggtgtagtcggtttattaaaggaagggagttgtggctgatgtggatagatatctttaaagctggcgactgcacccaacgagtgtggtggtagcttgttactgtatattcggtaagatatattttgtggggtttttagtggtgtttggtaggttagtgtctggtatatgagttgtaggcatgacaatttggaaaggggtggactttgggaatattgtgggatttcaataccttagtttgtacagggtaattgttacaaatgatacaaagaactgtatttcttttcatttgttttaattggttgtatatcaagtccgttagacgagctcagtgccatggcttttggcactgtatttcatttttagaggtacactacatccagtgaggtatggtaaggttgagggcataatgaaggcaccttgtactgacagtcacagacctctcaccgagaattttatgagatatactcgggttcattttaggctccgattcgattcaaattattactgtcgaaatcggttgagcatccgttgatttccgaacagatctggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgcttttcgtagataatggaatacaaatggatatccagagtatacacatggatagtatacactgacacgacaattctgtatctctttatgttaactactgtgaggcgttaaatagagcttgatatataaaatgttacatttcacagtctgaacttttgcagattacctaatttggtaagatattaattatgaactgaaagttgatggcatccctaaatttgatgaaagggggatcccccgggttcgaaggtaccaaggaagcatgcggtacccgaattcctgaggtgtctcacaagtgccgtgcagtcccgcccccacttgcttctctttgtgtgtagtgtacgtacattatcgagaccgttgttcccgcccacctcgatccggtctagactgaggtgtctcacaagtgccgtgcagtcccgcccccacttgcttctctttgtgtgtagtgtacgtacattatcgagaccgttgttcccgcccacctcgatccggggatccctgaggtgtctcacaagtgccgtgcagtcccgcccccacttgcttctctttgtgtgtagtgtacgtacattatcgagaccgttgttcccgcccacctcgatccgggtcgacctgaggtgtctcacaagtgccgtgcagtcccgcccccacttgcttctctttgtgtgtagtgtacgtacattatcgagaccgttgttcccgcccacctcgatccgggagctcctgaggtgtctcacaagtgccgtgcagtcccgcccccacttgcttctctttgtgtgtagtgtacgtacattatcgagaccgttgttcccgcccacctcgatccggtctagactgaggtgtctcacaagtgccgtgcagtcccgcccccacttgcttctctttgtgtgtagtgtacgtacattatcgagaccgttgttcccgcccacctcgatccggggatccctgaggtgtctcacaagtgccgtgcagtcccgcccccacttgcttctctttgtgtgtagtgtacgtacattatcgagaccgttgttcccgcccacctcgatccgggtcgacctgaggtgtctcacaagtgccgtgcagtcccgcccccacttgcttctctttgtgtgtagtgtacgtacattatcgagaccgttgttcccgcccacctcgatccgggcatgcctgcagaagcttttgtggttgggactttagccaagggtataaaagaccaccgtccccgaattacctttcctcttcttttctctctctccttgtcaactcacacccgaaatcgttaagcatttccttctgagtataagaatcattcaaaggcgcgccacaatggataagaaatactccattggcctggacatcggaaccaactccgtgggttgggccgtgatcaccgatgagtacaaggtgccctctaagaaattcaaggtcctgggcaacaccgaccgacactccatcaagaagaacctgatcggcgctctgctcttcgactctggcgagaccgctgaggccacccgactgaagcgaaccgctcgaagacgatacacccgaagaaagaaccgaatctgttacctgcaggagatcttctctaacgagatggccaaggtggacgactctttcttccaccgactggaggagtctttcctggtggaggaggacaagaagcacgagcgacaccccatcttcggcaacatcgtggacgaggtggcctaccacgagaagtaccccaccatctaccacctgcgaaagaagctggtggactctaccgacaaggccgacctgcgactgatctacctggccctggcccacatgatcaagttccgaggccacttcctgatcgagggcgacctgaaccccgacaactctgacgtggacaagctgttcatccagctggtgcagacctacaaccagctcttcgaagagaaccccattaacgcttctggcgtggatgctaaggccatcctgtctgcccgactgtctaagtctcgacgactcgagaacctgattgctcagctccccggagagaagaagaacggtctgttcggaaacctgattgctctgtccctgggtctcacccctaacttcaagtccaacttcgatctggctgaggacgctaagctgcagctgtctaaggacacctacgacgatgacctggataacctgctcgcccagattggcgaccagtacgccgacctgttcctggccgccaagaacctgtctgacgccatcctgctgtctgacatcctgcgagtgaacaccgagatcaccaaggcccccctgtctgcctccatgattaagcgatacgatgagcaccaccaggatctgaccctcctcaaggctctggtccgacagcagctgcccgagaagtacaaggagattttcttcgaccagtctaagaacggctacgccggctacatcgacggcggcgcctctcaggaggagttctacaagttcattaagcccatcctggagaagatggacggaaccgaggaactgctcgtgaagctgaaccgagaggacctcctgcgaaagcagcgaaccttcgacaacggctctatcccccaccagatccacctgggcgagctgcacgccatcctgcgacgacaggaggacttctaccccttcctgaaggacaaccgagagaagatcgagaagatcctgaccttccgaatcccctactacgtgggacccctggcccgaggaaactctcgattcgcttggatgacccgaaagtctgaggagaccattaccccctggaacttcgaggaggtggtggataagggcgcctctgctcagtctttcatcgagcgaatgaccaacttcgacaagaacctccccaacgagaaggtcctgcccaagcactctctgctctacgagtacttcaccgtctacaacgagctcaccaaggtcaagtacgtgaccgagggaatgcgaaagcccgctttcctgtctggagagcagaagaaggctattgtggatctgctcttcaagactaaccgaaaggtcaccgtcaagcagctgaaggaggattacttcaagaagattgagtgtttcgattctgtcgagatctccggcgtcgaggaccgattcaacgcctctctgggtacctaccacgacctgctgaagattatcaaggacaaggatttcctggataacgaggagaacgaggatattctcgaggacattgtcctgaccctcaccctgttcgaggatcgagagatgattgaggagcgactcaagacctacgctcacctgttcgacgacaaggtgatgaagcagctgaagcgacgacgatacaccggctggggccgactgtctcgaaagctgatcaacggcatccgagacaagcagtctggcaagaccatcctggacttcctgaagtctgacggcttcgccaaccgaaacttcatgcagctgatccacgacgactctctgaccttcaaggaggacatccagaaggcccaggtgtctggccagggcgactctctgcacgagcacatcgccaacctggccggctctcccgccattaagaaaggtatcctgcagaccgtcaaggtggtcgatgagctcgtcaaggtgatgggccgacacaagcccgagaacattgtcattgagatggctcgagagaaccagactactcagaagggtcagaaaaactcccgagagcgaatgaagcgaattgaggaaggtattaaggagctgggatcccagattctcaaggagcatcccgtggagaacactcagctccagaacgagaagctgtacctgtactatctgcagaacggtcgagacatgtacgtcgaccaggagctggatatcaaccgactctccgactacgatgtggaccacattgtgccccagtccttcctgaaggacgattctatcgataacaaggtgctgacccgatccgacaagaaccgaggcaagtctgacaacgtgccctccgaggaggtggtcaagaagatgaagaactactggcgacagctgctgaacgccaagctgattacccagcgaaagttcgacaacctgaccaaggccgagcgaggcggcctgtctgagctggacaaggccggcttcatcaagcgacagctggtggagacccgacagatcaccaagcacgtggcccagatcctggactctcgaatgaacaccaagtacgacgagaacgacaagctgatccgagaggtgaaggtgatcaccctgaagtctaagctggtgtctgacttccgaaaggacttccagttctacaaggtgcgagagattaacaactaccaccacgcccacgatgcctacctgaacgctgtcgtgggcaccgccctcatcaagaagtatcccaagctggagtccgagttcgtctacggcgactacaaggtctacgatgtgcgaaaaatgattgccaagtccgagcaggagattggcaaggctaccgccaagtacttcttctactccaacattatgaacttcttcaagaccgagattaccctggctaacggcgagattcgaaagcgacccctcattgagaccaacggagagaccggtgagatcgtgtgggacaagggacgagacttcgccaccgtgcgaaaggtgctgtctatgccccaggtgaacatcgtgaagaagaccgaggtgcagaccggaggtttctctaaggagtccatcctgcccaagcgaaactctgacaagctgatcgcccgaaagaaggactgggaccccaagaagtacggaggtttcgactctcccaccgtggcttactctgtgctggtggtggccaaggtggagaagggcaagtctaagaagctgaagtctgtgaaggagctgctgggcattaccatcatggagcgatcttctttcgagaagaaccccattgacttcctggaggccaagggatacaaggaggtgaagaaagatctgattatcaagctccccaagtactctctgttcgagctggagaacggacgaaagcgaatgctggcctctgccggcgagctgcagaagggaaacgagctggccctgccctccaagtacgtcaacttcctgtacctcgcctcccattacgagaagctgaagggctctcccgaggataacgagcagaagcagctcttcgtggagcagcataagcactacctggacgagatcatcgagcagatctctgagttctctaagcgagtgatcctggctgacgccaacctggataaggtgctgtctgcttacaacaagcaccgagacaagcccattcgagagcaggctgagaacatcattcacctgttcaccctgaccaacctgggagcccccgctgccttcaagtacttcgacaccaccatcgaccgaaagcgatacacctctaccaaggaggtgctggacgccaccctgatccaccagtctatcaccggcctgtacgagacccgaatcgacctgtctcagctgggcggcgactctcgagccgaccccaagaagaagcgaaaggtgtaagctagcctcatgtaattagttatgtcacgcttacattcacgccctccctccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgca

SEQ ID NO: 53 - DNA, 11883 bp, Artificial Sequence
  Nucleotide sequence encoding pCRISPYL-34749gRNA2
cacatttccccgaaaagtgccacctgacgtccccagttgcaaaagttgacacaactctagatctgcttccaaatatagaatcataacaagggttagggtgtgattatataatattggtcttaattgatgtgctagggctttaaaagttggttaaaataacgctctaatgcctttttaatatattgtctttttcaaaatctcaaatcggacacttcttcgtgtatgagactccattttttggctccgtcacgtgatatggggtcggcgcaggttgacgttattatcagctatagtggtgtaaacaaagttttttactagctgtaatggcattttgtcggagtggtaaatcgccttcttgttgtgcgttcgagttctggactctgcactgggctactttgaaaaatacctctaatgcgccgatggtttagtggtaaaatccatcgttgccatcgatgggcccccggttcgattccgggtcggcgcaggttgaccgggtcggcgcaggttgacgtaccagctcgctgagcagagtgttttagagctagaaatagcggttttagagctagaaatagcaagttaaaataaggctagtccgttatcaacttgaaaaagtggcaccgagtcggtgcttttttttacgtctaagaaaccattattatcatgacattaacctataaaaataggcgtatcacgaggcccagatcctctagagtcgaagcggccgctatgtctgataaaaggatgtaacataggcaagctgctcgtgagtgttgagtacgaaccttagatccaaatcacccgcacccacggatatacttgcttgaatatacagtagtatgcggccgcttcgacaccatatcatataaaactaacaatgcattgaattaatacgaagactacccgttgctatctccacaccgttatctccacggtccaaaggctgctcaatgtgctgcatacgtaacgtggggtgcaaccttgagcacatagtactttccgaaaaccggcgataattaagtgtgcactccaacttttcacactgagcataaaatgtggagaagaaatcacactaaaaagtcaggtagactggaaaatgcgccatgaaataatatctcttgctacagtaatgcccagcatcgaggggtattgtgtcaccaacactatagtggcagctgaagcgctcgtgattgtagtatgagtctttattggtgatgggaagagttcactcaatattctcgttactgccaaaacaccacggtaatcggccagacaccatggatgtagatcaccaagcctgtgaatgttattcgagctaaaatgcacatggttggtgaaaggagtagttgctgtcgaattccgtcgtcgcctgagtcatcatttatttaccagttggccacaaacccttgacgatctcgtatgtcccctccgacatactcccggccggctggggtacgttcgatagcgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgtttgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtctcctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacaccgagacctactccgttcctgaggttgagcgaattgcccgaatggccgccttcctggcccttcagcacaacccccctcttcccgtgtggtctcttgacaaggccaacgtgctggcctcctctcgactttggcgaaagactgtcactcgagtcctcaaggacgaattcccccagctcgagctcaaccaccagctgatcgactcggccgccatgatcctcatcaagcagccctccaagatgaatggtatcatcatcaccaccaacatgtttggcgatatcatctccgacgaggcctccgtcatccccggttctctgggtctgctgccctccgcctctctggcttctctgcccgacaccaacgaggcgttcggtctgtacgagccctgtcacggatctgcccccgatctcggcaagcagaaggtcaaccccattgccaccattctgtctgccgccatgatgctcaagttctctcttaacatgaagcccgccggtgacgctgttgaggctgccgtcaaggagtccgtcgaggctggtatcactaccgccgatatcggaggctcttcctccacctccgaggtcggagactttgttgccaacaaggtcaaggagctgctcaagaaggagtaagtcgtttctacgacgcattgatggaaggagcaaactgacgcgcctgcgggttggtctaccggcagggtccgctagtgtataagactctataaaaagggcccagccctgctaatgaaatgatgatttataatttaccggtgtagcaaccttgactagaagaagcagattgggtgtgtttgtagtggaggacagtggtacgttttggaaacagtcttcttgaaagtgtcttgtctacagtatattcactcataacctcaatagccaagggtgtagtcggtttattaaaggaagggagttgtggctgatgtggatagatatctttaaagctggcgactgcacccaacgagtgtggtggtagcttgttactgtatattcggtaagatatattttgtggggtttttagtggtgtttggtaggttagtgtctggtatatgagttgtaggcatgacaatttggaaaggggtggactttgggaatattgtgggatttcaataccttagtttgtacagggtaattgttacaaatgatacaaagaactgtatttcttttcatttgttttaattggttgtatatcaagtccgttagacgagctcagtgccatggcttttggcactgtatttcatttttagaggtacactacatccagtgaggtatggtaaggttgagggcataatgaaggcaccttgtactgacagtcacagacctctcaccgagaattttatgagatatactcgggttcattttaggctccgattcgattcaaattattactgtcgaaatcggttgagcatccgttgatttccgaacagatctggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgcttttcgtagataatggaatacaaatggatatccagagtatacacatggatagtatacactgacacgacaattctgtatctctttatgttaactactgtgaggcgttaaatagagcttgatatataaaatgttacatttcacagtctgaacttttgcagattacctaatttggtaagatattaattatgaactgaaagttgatggcatccctaaatttgatgaaagggggatcccccgggttcgaaggtaccaaggaagcatgcggtacccgaattcctgaggtgtctcacaagtgccgtgcagtcccgcccccacttgcttctctttgtgtgtagtgtacgtacattatcgagaccgttgttcccgcccacctcgatccggtctagactgaggtgtctcacaagtgccgtgcagtcccgcccccacttgcttctctttgtgtgtagtgtacgtacattatcgagaccgttgttcccgcccacctcgatccggggatccctgaggtgtctcacaagtgccgtgcagtcccgcccccacttgcttctctttgtgtgtagtgtacgtacattatcgagaccgttgttcccgcccacctcgatccgggtcgacctgaggtgtctcacaagtgccgtgcagtcccgcccccacttgcttctctttgtgtgtagtgtacgtacattatcgagaccgttgttcccgcccacctcgatccgggagctcctgaggtgtctcacaagtgccgtgcagtcccgcccccacttgcttctctttgtgtgtagtgtacgtacattatcgagaccgttgttcccgcccacctcgatccggtctagactgaggtgtctcacaagtgccgtgcagtcccgcccccacttgcttctctttgtgtgtagtgtacgtacattatcgagaccgttgttcccgcccacctcgatccggggatccctgaggtgtctcacaagtgccgtgcagtcccgcccccacttgcttctctttgtgtgtagtgtacgtacattatcgagaccgttgttcccgcccacctcgatccgggtcgacctgaggtgtctcacaagtgccgtgcagtcccgcccccacttgcttctctttgtgtgtagtgtacgtacattatcgagaccgttgttcccgcccacctcgatccgggcatgcctgcagaagcttttgtggttgggactttagccaagggtataaaagaccaccgtccccgaattacctttcctcttcttttctctctctccttgtcaactcacacccgaaatcgttaagcatttccttctgagtataagaatcattcaaaggcgcgccacaatggataagaaatactccattggcctggacatcggaaccaactccgtgggttgggccgtgatcaccgatgagtacaaggtgccctctaagaaattcaaggtcctgggcaacaccgaccgacactccatcaagaagaacctgatcggcgctctgctcttcgactctggcgagaccgctgaggccacccgactgaagcgaaccgctcgaagacgatacacccgaagaaagaaccgaatctgttacctgcaggagatcttctctaacgagatggccaaggtggacgactctttcttccaccgactggaggagtctttcctggtggaggaggacaagaagcacgagcgacaccccatcttcggcaacatcgtggacgaggtggcctaccacgagaagtaccccaccatctaccacctgcgaaagaagctggtggactctaccgacaaggccgacctgcgactgatctacctggccctggcccacatgatcaagttccgaggccacttcctgatcgagggcgacctgaaccccgacaactctgacgtggacaagctgttcatccagctggtgcagacctacaaccagctcttcgaagagaaccccattaacgcttctggcgtggatgctaaggccatcctgtctgcccgactgtctaagtctcgacgactcgagaacctgattgctcagctccccggagagaagaagaacggtctgttcggaaacctgattgctctgtccctgggtctcacccctaacttcaagtccaacttcgatctggctgaggacgctaagctgcagctgtctaaggacacctacgacgatgacctggataacctgctcgcccagattggcgaccagtacgccgacctgttcctggccgccaagaacctgtctgacgccatcctgctgtctgacatcctgcgagtgaacaccgagatcaccaaggcccccctgtctgcctccatgattaagcgatacgatgagcaccaccaggatctgaccctcctcaaggctctggtccgacagcagctgcccgagaagtacaaggagattttcttcgaccagtctaagaacggctacgccggctacatcgacggcggcgcctctcaggaggagttctacaagttcattaagcccatcctggagaagatggacggaaccgaggaactgctcgtgaagctgaaccgagaggacctcctgcgaaagcagcgaaccttcgacaacggctctatcccccaccagatccacctgggcgagctgcacgccatcctgcgacgacaggaggacttctaccccttcctgaaggacaaccgagagaagatcgagaagatcctgaccttccgaatcccctactacgtgggacccctggcccgaggaaactctcgattcgcttggatgacccgaaagtctgaggagaccattaccccctggaacttcgaggaggtggtggataagggcgcctctgctcagtctttcatcgagcgaatgaccaacttcgacaagaacctccccaacgagaaggtcctgcccaagcactctctgctctacgagtacttcaccgtctacaacgagctcaccaaggtcaagtacgtgaccgagggaatgcgaaagcccgctttcctgtctggagagcagaagaaggctattgtggatctgctcttcaagactaaccgaaaggtcaccgtcaagcagctgaaggaggattacttcaagaagattgagtgtttcgattctgtcgagatctccggcgtcgaggaccgattcaacgcctctctgggtacctaccacgacctgctgaagattatcaaggacaaggatttcctggataacgaggagaacgaggatattctcgaggacattgtcctgaccctcaccctgttcgaggatcgagagatgattgaggagcgactcaagacctacgctcacctgttcgacgacaaggtgatgaagcagctgaagcgacgacgatacaccggctggggccgactgtctcgaaagctgatcaacggcatccgagacaagcagtctggcaagaccatcctggacttcctgaagtctgacggcttcgccaaccgaaacttcatgcagctgatccacgacgactctctgaccttcaaggaggacatccagaaggcccaggtgtctggccagggcgactctctgcacgagcacatcgccaacctggccggctctcccgccattaagaaaggtatcctgcagaccgtcaaggtggtcgatgagctcgtcaaggtgatgggccgacacaagcccgagaacattgtcattgagatggctcgagagaaccagactactcagaagggtcagaaaaactcccgagagcgaatgaagcgaattgaggaaggtattaaggagctgggatcccagattctcaaggagcatcccgtggagaacactcagctccagaacgagaagctgtacctgtactatctgcagaacggtcgagacatgtacgtcgaccaggagctggatatcaaccgactctccgactacgatgtggaccacattgtgccccagtccttcctgaaggacgattctatcgataacaaggtgctgacccgatccgacaagaaccgaggcaagtctgacaacgtgccctccgaggaggtggtcaagaagatgaagaactactggcgacagctgctgaacgccaagctgattacccagcgaaagttcgacaacctgaccaaggccgagcgaggcggcctgtctgagctggacaaggccggcttcatcaagcgacagctggtggagacccgacagatcaccaagcacgtggcccagatcctggactctcgaatgaacaccaagtacgacgagaacgacaagctgatccgagaggtgaaggtgatcaccctgaagtctaagctggtgtctgacttccgaaaggacttccagttctacaaggtgcgagagattaacaactaccaccacgcccacgatgcctacctgaacgctgtcgtgggcaccgccctcatcaagaagtatcccaagctggagtccgagttcgtctacggcgactacaaggtctacgatgtgcgaaaaatgattgccaagtccgagcaggagattggcaaggctaccgccaagtacttcttctactccaacattatgaacttcttcaagaccgagattaccctggctaacggcgagattcgaaagcgacccctcattgagaccaacggagagaccggtgagatcgtgtgggacaagggacgagacttcgccaccgtgcgaaaggtgctgtctatgccccaggtgaacatcgtgaagaagaccgaggtgcagaccggaggtttctctaaggagtccatcctgcccaagcgaaactctgacaagctgatcgcccgaaagaaggactgggaccccaagaagtacggaggtttcgactctcccaccgtggcttactctgtgctggtggtggccaaggtggagaagggcaagtctaagaagctgaagtctgtgaaggagctgctgggcattaccatcatggagcgatcttctttcgagaagaaccccattgacttcctggaggccaagggatacaaggaggtgaagaaagatctgattatcaagctccccaagtactctctgttcgagctggagaacggacgaaagcgaatgctggcctctgccggcgagctgcagaagggaaacgagctggccctgccctccaagtacgtcaacttcctgtacctcgcctcccattacgagaagctgaagggctctcccgaggataacgagcagaagcagctcttcgtggagcagcataagcactacctggacgagatcatcgagcagatctctgagttctctaagcgagtgatcctggctgacgccaacctggataaggtgctgtctgcttacaacaagcaccgagacaagcccattcgagagcaggctgagaacatcattcacctgttcaccctgaccaacctgggagcccccgctgccttcaagtacttcgacaccaccatcgaccgaaagcgatacacctctaccaaggaggtgctggacgccaccctgatccaccagtctatcaccggcctgtacgagacccgaatcgacctgtctcagctgggcggcgactctcgagccgaccccaagaagaagcgaaaggtgtaagctagcctcatgtaattagttatgtcacgcttacattcacgccctccctccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgca

SEQ ID NO: 54 - DNA, 11883 bp, Artificial Sequence
  Nucleotide sequence encoding pCRISPYL-CTA1
cacatttccccgaaaagtgccacctgacgtccccagttgcaaaagttgacacaactctagatctgcttccaaatatagaatcataacaagggttagggtgtgattatataatattggtcttaattgatgtgctagggctttaaaagttggttaaaataacgctctaatgcctttttaatatattgtctttttcaaaatctcaaatcggacacttcttcgtgtatgagactccattttttggctccgtcacgtgatatggggtcggcgcaggttgacgttattatcagctatagtggtgtaaacaaagttttttactagctgtaatggcattttgtcggagtggtaaatcgccttcttgttgtgcgttcgagttctggactctgcactgggctactttgaaaaatacctctaatgcgccgatggtttagtggtaaaatccatcgttgccatcgatgggcccccggttcgattccgggtcggcgcaggttgaccgggtcggcgcaggttgacgtgccgagtcaagacgaaactagttttagagctagaaatagcggttttagagctagaaatagcaagttaaaataaggctagtccgttatcaacttgaaaaagtggcaccgagtcggtgcttttttttacgtctaagaaaccattattatcatgacattaacctataaaaataggcgtatcacgaggcccagatcctctagagtcgaagcggccgctatgtctgataaaaggatgtaacataggcaagctgctcgtgagtgttgagtacgaaccttagatccaaatcacccgcacccacggatatacttgcttgaatatacagtagtatgcggccgcttcgacaccatatcatataaaactaacaatgcattgaattaatacgaagactacccgttgctatctccacaccgttatctccacggtccaaaggctgctcaatgtgctgcatacgtaacgtggggtgcaaccttgagcacatagtactttccgaaaaccggcgataattaagtgtgcactccaacttttcacactgagcataaaatgtggagaagaaatcacactaaaaagtcaggtagactggaaaatgcgccatgaaataatatctcttgctacagtaatgcccagcatcgaggggtattgtgtcaccaacactatagtggcagctgaagcgctcgtgattgtagtatgagtctttattggtgatgggaagagttcactcaatattctcgttactgccaaaacaccacggtaatcggccagacaccatggatgtagatcaccaagcctgtgaatgttattcgagctaaaatgcacatggttggtgaaaggagtagttgctgtcgaattccgtcgtcgcctgagtcatcatttatttaccagttggccacaaacccttgacgatctcgtatgtcccctccgacatactcccggccggctggggtacgttcgatagcgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgtttgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtctcctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacaccgagacctactccgttcctgaggttgagcgaattgcccgaatggccgccttcctggcccttcagcacaacccccctcttcccgtgtggtctcttgacaaggccaacgtgctggcctcctctcgactttggcgaaagactgtcactcgagtcctcaaggacgaattcccccagctcgagctcaaccaccagctgatcgactcggccgccatgatcctcatcaagcagccctccaagatgaatggtatcatcatcaccaccaacatgtttggcgatatcatctccgacgaggcctccgtcatccccggttctctgggtctgctgccctccgcctctctggcttctctgcccgacaccaacgaggcgttcggtctgtacgagccctgtcacggatctgcccccgatctcggcaagcagaaggtcaaccccattgccaccattctgtctgccgccatgatgctcaagttctctcttaacatgaagcccgccggtgacgctgttgaggctgccgtcaaggagtccgtcgaggctggtatcactaccgccgatatcggaggctcttcctccacctccgaggtcggagactttgttgccaacaaggtcaaggagctgctcaagaaggagtaagtcgtttctacgacgcattgatggaaggagcaaactgacgcgcctgcgggttggtctaccggcagggtccgctagtgtataagactctataaaaagggcccagccctgctaatgaaatgatgatttataatttaccggtgtagcaaccttgactagaagaagcagattgggtgtgtttgtagtggaggacagtggtacgttttggaaacagtcttcttgaaagtgtcttgtctacagtatattcactcataacctcaatagccaagggtgtagtcggtttattaaaggaagggagttgtggctgatgtggatagatatctttaaagctggcgactgcacccaacgagtgtggtggtagcttgttactgtatattcggtaagatatattttgtggggtttttagtggtgtttggtaggttagtgtctggtatatgagttgtaggcatgacaatttggaaaggggtggactttgggaatattgtgggatttcaataccttagtttgtacagggtaattgttacaaatgatacaaagaactgtatttcttttcatttgttttaattggttgtatatcaagtccgttagacgagctcagtgccatggcttttggcactgtatttcatttttagaggtacactacatccagtgaggtatggtaaggttgagggcataatgaaggcaccttgtactgacagtcacagacctctcaccgagaattttatgagatatactcgggttcattttaggctccgattcgattcaaattattactgtcgaaatcggttgagcatccgttgatttccgaacagatctggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgcttttcgtagataatggaatacaaatggatatccagagtatacacatggatagtatacactgacacgacaattctgtatctctttatgttaactactgtgaggcgttaaatagagcttgatatataaaatgttacatttcacagtctgaacttttgcagattacctaatttggtaagatattaattatgaactgaaagttgatggcatccctaaatttgatgaaagggggatcccccgggttcgaaggtaccaaggaagcatgcggtacccgaattcctgaggtgtctcacaagtgccgtgcagtcccgcccccacttgcttctctttgtgtgtagtgtacgtacattatcgagaccgttgttcccgcccacctcgatccggtctagactgaggtgtctcacaagtgccgtgcagtcccgcccccacttgcttctctttgtgtgtagtgtacgtacattatcgagaccgttgttcccgcccacctcgatccggggatccctgaggtgtctcacaagtgccgtgcagtcccgcccccacttgcttctctttgtgtgtagtgtacgtacattatcgagaccgttgttcccgcccacctcgatccgggtcgacctgaggtgtctcacaagtgccgtgcagtcccgcccccacttgcttctctttgtgtgtagtgtacgtacattatcgagaccgttgttcccgcccacctcgatccgggagctcctgaggtgtctcacaagtgccgtgcagtcccgcccccacttgcttctctttgtgtgtagtgtacgtacattatcgagaccgttgttcccgcccacctcgatccggtctagactgaggtgtctcacaagtgccgtgcagtcccgcccccacttgcttctctttgtgtgtagtgtacgtacattatcgagaccgttgttcccgcccacctcgatccggggatccctgaggtgtctcacaagtgccgtgcagtcccgcccccacttgcttctctttgtgtgtagtgtacgtacattatcgagaccgttgttcccgcccacctcgatccgggtcgacctgaggtgtctcacaagtgccgtgcagtcccgcccccacttgcttctctttgtgtgtagtgtacgtacattatcgagaccgttgttcccgcccacctcgatccgggcatgcctgcagaagcttttgtggttgggactttagccaagggtataaaagaccaccgtccccgaattacctttcctcttcttttctctctctccttgtcaactcacacccgaaatcgttaagcatttccttctgagtataagaatcattcaaaggcgcgccacaatggataagaaatactccattggcctggacatcggaaccaactccgtgggttgggccgtgatcaccgatgagtacaaggtgccctctaagaaattcaaggtcctgggcaacaccgaccgacactccatcaagaagaacctgatcggcgctctgctcttcgactctggcgagaccgctgaggccacccgactgaagcgaaccgctcgaagacgatacacccgaagaaagaaccgaatctgttacctgcaggagatcttctctaacgagatggccaaggtggacgactctttcttccaccgactggaggagtctttcctggtggaggaggacaagaagcacgagcgacaccccatcttcggcaacatcgtggacgaggtggcctaccacgagaagtaccccaccatctaccacctgcgaaagaagctggtggactctaccgacaaggccgacctgcgactgatctacctggccctggcccacatgatcaagttccgaggccacttcctgatcgagggcgacctgaaccccgacaactctgacgtggacaagctgttcatccagctggtgcagacctacaaccagctcttcgaagagaaccccattaacgcttctggcgtggatgctaaggccatcctgtctgcccgactgtctaagtctcgacgactcgagaacctgattgctcagctccccggagagaagaagaacggtctgttcggaaacctgattgctctgtccctgggtctcacccctaacttcaagtccaacttcgatctggctgaggacgctaagctgcagctgtctaaggacacctacgacgatgacctggataacctgctcgcccagattggcgaccagtacgccgacctgttcctggccgccaagaacctgtctgacgccatcctgctgtctgacatcctgcgagtgaacaccgagatcaccaaggcccccctgtctgcctccatgattaagcgatacgatgagcaccaccaggatctgaccctcctcaaggctctggtccgacagcagctgcccgagaagtacaaggagattttcttcgaccagtctaagaacggctacgccggctacatcgacggcggcgcctctcaggaggagttctacaagttcattaagcccatcctggagaagatggacggaaccgaggaactgctcgtgaagctgaaccgagaggacctcctgcgaaagcagcgaaccttcgacaacggctctatcccccaccagatccacctgggcgagctgcacgccatcctgcgacgacaggaggacttctaccccttcctgaaggacaaccgagagaagatcgagaagatcctgaccttccgaatcccctactacgtgggacccctggcccgaggaaactctcgattcgcttggatgacccgaaagtctgaggagaccattaccccctggaacttcgaggaggtggtggataagggcgcctctgctcagtctttcatcgagcgaatgaccaacttcgacaagaacctccccaacgagaaggtcctgcccaagcactctctgctctacgagtacttcaccgtctacaacgagctcaccaaggtcaagtacgtgaccgagggaatgcgaaagcccgctttcctgtctggagagcagaagaaggctattgtggatctgctcttcaagactaaccgaaaggtcaccgtcaagcagctgaaggaggattacttcaagaagattgagtgtttcgattctgtcgagatctccggcgtcgaggaccgattcaacgcctctctgggtacctaccacgacctgctgaagattatcaaggacaaggatttcctggataacgaggagaacgaggatattctcgaggacattgtcctgaccctcaccctgttcgaggatcgagagatgattgaggagcgactcaagacctacgctcacctgttcgacgacaaggtgatgaagcagctgaagcgacgacgatacaccggctggggccgactgtctcgaaagctgatcaacggcatccgagacaagcagtctggcaagaccatcctggacttcctgaagtctgacggcttcgccaaccgaaacttcatgcagctgatccacgacgactctctgaccttcaaggaggacatccagaaggcccaggtgtctggccagggcgactctctgcacgagcacatcgccaacctggccggctctcccgccattaagaaaggtatcctgcagaccgtcaaggtggtcgatgagctcgtcaaggtgatgggccgacacaagcccgagaacattgtcattgagatggctcgagagaaccagactactcagaagggtcagaaaaactcccgagagcgaatgaagcgaattgaggaaggtattaaggagctgggatcccagattctcaaggagcatcccgtggagaacactcagctccagaacgagaagctgtacctgtactatctgcagaacggtcgagacatgtacgtcgaccaggagctggatatcaaccgactctccgactacgatgtggaccacattgtgccccagtccttcctgaaggacgattctatcgataacaaggtgctgacccgatccgacaagaaccgaggcaagtctgacaacgtgccctccgaggaggtggtcaagaagatgaagaactactggcgacagctgctgaacgccaagctgattacccagcgaaagttcgacaacctgaccaaggccgagcgaggcggcctgtctgagctggacaaggccggcttcatcaagcgacagctggtggagacccgacagatcaccaagcacgtggcccagatcctggactctcgaatgaacaccaagtacgacgagaacgacaagctgatccgagaggtgaaggtgatcaccctgaagtctaagctggtgtctgacttccgaaaggacttccagttctacaaggtgcgagagattaacaactaccaccacgcccacgatgcctacctgaacgctgtcgtgggcaccgccctcatcaagaagtatcccaagctggagtccgagttcgtctacggcgactacaaggtctacgatgtgcgaaaaatgattgccaagtccgagcaggagattggcaaggctaccgccaagtacttcttctactccaacattatgaacttcttcaagaccgagattaccctggctaacggcgagattcgaaagcgacccctcattgagaccaacggagagaccggtgagatcgtgtgggacaagggacgagacttcgccaccgtgcgaaaggtgctgtctatgccccaggtgaacatcgtgaagaagaccgaggtgcagaccggaggtttctctaaggagtccatcctgcccaagcgaaactctgacaagctgatcgcccgaaagaaggactgggaccccaagaagtacggaggtttcgactctcccaccgtggcttactctgtgctggtggtggccaaggtggagaagggcaagtctaagaagctgaagtctgtgaaggagctgctgggcattaccatcatggagcgatcttctttcgagaagaaccccattgacttcctggaggccaagggatacaaggaggtgaagaaagatctgattatcaagctccccaagtactctctgttcgagctggagaacggacgaaagcgaatgctggcctctgccggcgagctgcagaagggaaacgagctggccctgccctccaagtacgtcaacttcctgtacctcgcctcccattacgagaagctgaagggctctcccgaggataacgagcagaagcagctcttcgtggagcagcataagcactacctggacgagatcatcgagcagatctctgagttctctaagcgagtgatcctggctgacgccaacctggataaggtgctgtctgcttacaacaagcaccgagacaagcccattcgagagcaggctgagaacatcattcacctgttcaccctgaccaacctgggagcccccgctgccttcaagtacttcgacaccaccatcgaccgaaagcgatacacctctaccaaggaggtgctggacgccaccctgatccaccagtctatcaccggcctgtacgagacccgaatcgacctgtctcagctgggcggcgactctcgagccgaccccaagaagaagcgaaaggtgtaagctagcctcatgtaattagttatgtcacgcttacattcacgccctccctccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgca

SEQ ID NO: 55 - DNA, 6731 bp, Artificial Sequence
  Nucleotide sequence encoding pRS-pTef2-AmVHPO-SKL
gaaccgtaaaaaggccgcgttgctggcgtttttccataggctcggcccccctgacgagcatcacaaaaatcgacgctcaagtcagaggtggcgaaacccgacaggactataaagataccaggcgttcccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactgcccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgaaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccacgcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgggccgtatacttacatatagtagatgtcaagcgtaggcgcttcccctgccggctgtgagggcgccataaccaaggtatctatagaccgccaatcagcaaactacctccgtacattcatgttgcacccacacatttatacacccagaccgcgacaaattacccataaggttgtttgtgacggcgtcgtacaagagaacgtgggaactttttaggctcaccaaaaaagaaagaaaaaatacgagttgctgacagaagcctcaagaaaaaaaaaattcttcttcgactatgctggaggcagagatgatcgagccggtagttaactatatatagctaaattggttccatcaccttcttttctggtgtcgctccttctagtgctatttctggcttttcctatttttttttttccatttttctttctctctttctaatatataaattctcttgcattttctatttttctctctatctattctacttgtttattcccttcaaggtttttttttaaggagtacttgtttttagaatatacggtcaacgaactataattaactaaacatgaacactaggagacaacaagcccaaaacatcagaaacaatgccgctgaattggctgctaatagacctcacccacaacatataaataacaaagaggaatacgaatataggagaccaaaaaaagacggcaacgagccttcacacattgctaatttcaccaaggggttgcctcacgacgaacatacaggcttattactaaattccgccgactatgaccagttcgtcttaggtattcaatctggagacacgacggattttgctaggacgccattaggtcctgccgaattaccaaaagtacatggttgtttgtcaaaacaaaagattgactgtgatgatgaccacaggtctggattctggaagtcacaaatagctcaaggcgctgcgggtggtgatggagccaaacttagggcttgggaaagtgctggtgctggtttagtttttgacttagagggtccagatgcccaggcagtaacaatgccccctgctccgagattagaaagtccagagcttacttcagaaatagcagaagtgtattcacaagcattgctcagagacattcatttctctcagttacgtgaccctggcttgggagatcaagtaaatgcttgtgattcttgcccaacccaactttctatttatgaagcaatagatattctaaatactgtccagattgaaggccaaaattggttttcggccaattgttgtgatctcacagatgacgagcaggctcgtcagagaccactcgtaactcgacaaaatatatttcgtggcatcgcacctggagatgatgtcggtccctatctatcccagtttctgctgattggtaacaatgcgctgggaggtggtgtatttgggcaagaagccggccatatcggatatggtgctataagaattgatcagagagtcagaaaggctaccccctgcaaagactttatgactaacttcgaaacttggttggatgtgcaaaatggtgcagatttgaggggtctagaaacctacgttgacgctgatccgggaaaatgtagggaatttccagcgtatagagtcatcactacacctcgggacttagcaacctatgttcactacgatgcactatatgaggcatatttaaacgcttgcttgattttgctgggtatgggtgctccatttgaccccggtataccgttccaaaaaccagatgtggaagataaacagcaagggtttgcccattttggaggaccgcagattttgacactagtttgtgaggccgcaacaagagggttaaaagcagttagattccaaaagttcaacgttcatcgtcgtttgcgcccagaagctctaggtggtttagttgatagatataagcatggtaaaggtgcgggtgatgaacttaaacccgttgcagccttggtagaagcgctagagaatgttggtttgttgtcaaaggttgtagcccataatcaattgcaaaaccagaatcttgaccgcagtggcgatccatccagcgcaggggataattacttcctaccaatggccttcccagaaggttctcccatgcaccctagttatggtgcaggacatgcaactgtggctggagcttgtgtcactatgcttaaagcattttttgatcatggttggcaactgaatttgggtatggcgaatgggaaatacatatcctacgaacctaatcaagatgggagctcgcttcaacaagttcttttagattgccctctgacagtggaaggtgagttgaacaagattgcagctaacatctctattggtagagattgggctggcgtgcattactttacggattacatcgaatcgttacgattaggcgaaaagattgcgatcggcatattagaagagcaaaagctgacctacggagagaactttacaatgactgttccattgtacgatggaggcagcattcaaatcttgggaagaggtaggcgctccaaactttaagaattctgcagatatccatcacactggcggccgctcgagcatgcatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgc

SEQ ID NO: 56 - DNA, 7760 bp, Artificial Sequence
  Nucleotide sequence encoding pYES2-AmVHPO-SKL
atggggcgctacagggcgcgtggggatgatccactagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttatgaacactaggagacaacaagcccaaaacatcagaaacaatgccgctgaattggctgctaatagacctcacccacaacatataaataacaaagaggaatacgaatataggagaccaaaaaaagacggcaacgagccttcacacattgctaatttcaccaaggggttgcctcacgacgaacatacaggcttattactaaattccgccgactatgaccagttcgtcttaggtattcaatctggagacacgacggattttgctaggacgccattaggtcctgccgaattaccaaaagtacatggttgtttgtcaaaacaaaagattgactgtgatgatgaccacaggtctggattctggaagtcacaaatagctcaaggcgctgcgggtggtgatggagccaaacttagggcttgggaaagtgctggtgctggtttagtttttgacttagagggtccagatgcccaggcagtaacaatgccccctgctccgagattagaaagtccagagcttacttcagaaatagcagaagtgtattcacaagcattgctcagagacattcatttctctcagttacgtgaccctggcttgggagatcaagtaaatgcttgtgattcttgcccaacccaactttctatttatgaagcaatagatattctaaatactgtccagattgaaggccaaaattggttttcggccaattgttgtgatctcacagatgacgagcaggctcgtcagagaccactcgtaactcgacaaaatatatttcgtggcatcgcacctggagatgatgtcggtccctatctatcccagtttctgctgattggtaacaatgcgctgggaggtggtgtatttgggcaagaagccggccatatcggatatggtgctataagaattgatcagagagtcagaaaggctaccccctgcaaagactttatgactaacttcgaaacttggttggatgtgcaaaatggtgcagatttgaggggtctagaaacctacgttgacgctgatccgggaaaatgtagggaatttccagcgtatagagtcatcactacacctcgggacttagcaacctatgttcactacgatgcactatatgaggcatatttaaacgcttgcttgattttgctgggtatgggtgctccatttgaccccggtataccgttccaaaaaccagatgtggaagataaacagcaagggtttgcccattttggaggaccgcagattttgacactagtttgtgaggccgcaacaagagggttaaaagcagttagattccaaaagttcaacgttcatcgtcgtttgcgcccagaagctctaggtggtttagttgatagatataagcatggtaaaggtgcgggtgatgaacttaaacccgttgcagccttggtagaagcgctagagaatgttggtttgttgtcaaaggttgtagcccataatcaattgcaaaaccagaatcttgaccgcagtggcgatccatccagcgcaggggataattacttcctaccaatggccttcccagaaggttctcccatgcaccctagttatggtgcaggacatgcaactgtggctggagcttgtgtcactatgcttaaagcattttttgatcatggttggcaactgaatttgggtatggcgaatgggaaatacatatcctacgaacctaatcaagatgggagctcgcttcaacaagttcttttagattgccctctgacagtggaaggtgagttgaacaagattgcagctaacatctctattggtagagattgggctggcgtgcattactttacggattacatcgaatcgttacgattaggcgaaaagattgcgatcggcatattagaagagcaaaagctgacctacggagagaactttacaatgactgttccattgtacgatggaggcagcattcaaatcttgggaagaggtaggcgctccaaactttaagaattctgcagatatccatcacactggcggccgctcgagcatgcatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccattcagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgctta

SEQ ID NO: 57 - DNA, 8461 bp, Artificial Sequence
  Nucleotide sequence encoding pYLHR-34265gRNA1
ctattgaggaggccaagcgataccagaaggctggctgggaggcttaccagaagattaactgttagaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacaccaaaaccatgatcaaaccacccatggacttcctggaggcagaagaacttgttatggaaaagctcaagagagagaagccaagatactatcaagacatgtgtcgcaacttcaaggaggaccaagctctgtacaccgagaaacaggcctttgtcgacgatctggccccgcttttcgtagataatggaatacaaatggatatccagagtatacacatggatagtatacactgacacgacaattctgtatctctttatgttaactactgtgaggcgttaaatagagcttgatatataaaatgttacatttcacagtctgaacttttgcagattacctaatttggtaagatattaattatgaactgaaagttgatggcatccctaaatttgatgaaagttttcgagaaaatgagaacaactcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccattgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaaggccggcctgcgcacagtacggaccacagaaggctcgaaaagcagcatggagtgatgatttccgaagcgacagtgtgttgcaatcctttttgtgtctgtgtctcctgacaagtgccccaacctcattggatgctctttctctcttcaccaccagttgaagctaaatcacatccagttgggggtgtctcggggctggcgtaacaaaatgcagacggatgcagagtggatgccttggtggatgtggggggtctttgtccacggaagtgggtaatcactatcgagctacttctggtggtactgtagtacaagtcgactgtgtccagcacaagtcacaaccggttttgagtgagaagctttgttccaccttccaaactcgttcgacacttctaattgcagccttttcatctcatcgcagtcatcacagacctgcttaagcgagcctgtctccctgtgcacagtatgtacaagtagggttgctaattctatttgggaggtgacatccataagctcagaactggtgccttgatgcccagtactcgtaacccgcgggatactcaacctagatgacgtgcggcggcttctggcctttgtgtgttgaaattcttgtacatattggaagtgtagtgtggtggtggcggcggtgtacatccaggtgtacatccaatccttatccctggatcattgcactcctcagagtctacatgacccggctggttggtgtctccactacgacacaattctatgctgcagacccacacccaaaagcaggcctgtcgcacctcattgaacacggtcacctccctccaaagcacaggatgccgcctgaatagcaatacttctcctctcattggaccccggctgcaattggctttaccgtttgatctgtaacgttgttcagggaaccgtggcgcaggtggtctgcaacctagatgattgctatctggccatatttgggcggcggatccgtatataaagcggagtgtagctgtagtcagcccccacgatcaatccatcaggcgcccacgtgggtctctacgggggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaagaatgagagactttaaacatatgaatactcgccgtcagcaggcccaaaacatccgcaacaacgccgctgagctggccgctaatcgacctcaccctcagcacatcaacaacaaggaagagtacgagtacagacgaccaaagaaggacggcaacgagcccagccacattgctaacttcaccaagggcttgccccacgacgagcatacaggcctgctgctcaactccgccgactatgaccagttcgtcctgggcattcagtctggagacacgacggactttgctcgaaccccactcggacccgccgagctgcctaaggttcatggctgtctgagcaagcagaagattgactgtgatgacgaccaccgatctggattctggaagtcacagatcgctcaaggagctgcgggtggtgacggagccaagcttagggcttgggagagtgcgggggctggtctggtttttgacctcgaaggccccgacgcccaggcagtaacgatgccccccgccccgagactggagtcacctgagcttacttcagagatcgcagaggtttattcccaggccttgctccgggacatccacttctctcagctgcgagatcctggcttgggagaccaggtgaacgcctgtgattcttgcccaacccagctttctatttacgaggccattgatattctcaatactgtccagatcgagggccaaaactggttctctgccaactgctgcgatctcacagacgacgagcaggcacgacagcgacccctcgtaactcgacagaatatctttcggggcatcgctcctggagacgatgtcggtccctacctatcccagtttctgctgattggtaataacgcgctgggaggtggggtctttggccaggaagccggccatatcggatacggtgcaatacgaattgaccagcgagtcagaaaggccaccccctgcaaggacttcatgactaacttcgagacatggctggacgtgcagaacggtgcagatttgcgaggtctcgaaacctacgtggacgctgatccgggaaaatgtcgtgagtttcccgcctaccgagtcatcaccacgccccgggacctagccacctacgttcactacgatgcgctatatgaagcttatctgaacgcttgcctgattttgctgggaatgggagctcctttcgaccccggcattccgttccaaaagcccgacgtggaggacaaacagcagggctttgcccactttggaggaccgcaaatcttaaccctagtgtgtgaggccgccaccagaggccttaaagcagttcgcttccagaagttcaacgtgcaccgtagactgcgacctgaggctctcggtggtttggttgatcggtataagcacggtaagggtgcgggtgatgaacttaagcccgtcgcagccctggtcgaagcgctcgagaacgttggactcctctcgaaggtggtggcccataaccaactccagaaccagaaccttgaccgttccggcgacccctcctcggctggggataattacttcctcccaatggccttcccagaaggatcgcctatgcaccctagttacggtgccggacatgcaactgtggctggagcctgtgtcactatgcttaaagctttctttgatcatggttggcagctgaatttgggcatggcgaacggcaaatacatctcctacgagcctaaccaggacgggtcgtcgcttcagcaagtgcttctggattgccctctcacagtggagggagagttgaacaagattgctgctaacatctctattggacgggattgggctggcgtgcattacttcaccgactacatcgagtccctgcgactgggcgaaaagattgccatcggcattctggaagagcaaaagctgacctacggagagaactttacaatgaccgtccccctctacgatggcggctccattcaaatctctaagctgtaagaattcaccggttgtctgtggtatctaagctatttatcactctttacaacttctacctcaactatctactttaataaatgaatatcgtttattctctatgattactgtatatgcgttcctctaagacatctagagtccaaagaactctacgccaactccaaggacgacgctgtctactcctactcctttggagttccttacccccaccatccctactcatctcagcgggccggccctaccggccctctgctgctccaggacacctatctgattgacgctctggcccatttcgaccgagagcgaatccccgagcgagtcgtccatgccaacggaggaggagcccatggctactttgaggtgaccgatgacatctccgacatcacatacgccgagcccttccagaagattggctacaaatgccccaccaccgtgcgattttctaccgttggtggtgagcgaggctctcccgacactgctcgagacccccgaggtttcgctgtcaagcacaagaccgactggggtaactgggacatggttggtctcaactctcctgttttcttcattcgagatcctgtcaagttcatccatgtcaaccactctcagaagcgagacccccagaccaacctgactgccggagatgatgcctctaactactggaactatctggtccagaaccccgagtctctgcatcaggtggtgtacatgtttggagaccgaggtacccccaatggatggcgacacatgaactgcttctccactcatacttacaagatgatcaacaaggagggcaagctgacctacgtccagttccactacaagtccgaccagggagtcaagaacttcaccggacccgaggctgctgagatggctggaaagagcccggaccacgaccagaaggatctcttctttgccatcgataacggagactacccctcctggaccgtttcccttcagaccatgactcccgagcaggccgaggaatgggaatactccattctggatatgaccaagacctggccctatgacaagttccctctgcgaaaggtcggaaagctggtgctcaacaagaacgccgagaacttctttgaggagattgagcaggccgccttctctccctccaacctgatccacggtatcgaagcctctgatgatcctgttaattaagctagcgtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatc

SEQ ID NO: 58 - DNA, 8461 bp, Artificial Sequence
  Nucleotide sequence encoding pYLHR34265gRNA2
ctattgaggaggccaagcgataccagaaggctggctgggaggcttaccagaagattaactgttagaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacaccaaaaccatgatcaaaccacccatggacttcctggaggcagaagaacttgttatggaaaagctcaagagagagaagccaagatactatcaagacatgtgtcgcaacttcaaggaggaccaagctctgtacaccgagaaacaggcctttgtcgacgatctggccccgcttttcgtagataatggaatacaaatggatatccagagtatacacatggatagtatacactgacacgacaattctgtatctctttatgttaactactgtgaggcgttaaatagagcttgatatataaaatgttacatttcacagtctgaacttttgcagattacctaatttggtaagatattaattatgaactgaaagttgatggcatccctaaatttgatgaaagttttcgagaaaatgagaacaactcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccattgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaaggccggcctgcgcacagtacggaccacagaaggctcgaaaagcagcatggagtgatgatttccgaagcgacagtgtgttgcaatcctttttgtgtctgtgtctcctgacaagtgccccaacctcattggatgctctttctctcttcaccaccagttgaagctaaatcacatccagttgggggtgtctcggggctggcgtaacaaaatgcagacggatgcagagtggatgccttggtggatgtggggggtctttgtccacggaagtgggtaatcactatcgagctacttctggtggtactgtagtacaagtcgactgtgtccagcacaagtcacaaccggttttgagtgagaagctttgttccaccttccaaactcgttcgacacttctaattgcagccttttcatctcatcgcagtcatcacagacctgcttaagcgagcctgtctccctgtgcacagtatgtacaagtagggttgctaattctatttgggaggtgacatccataagctcagaactggtgccttgatgcccagtactcgtaacccgcgggatactcaacctagatgacgtgcggcggcttctggcctttgtgtgttgaaattcttgtacatattggaagtgtagtgtggtggtggcggcggtgtacatccaggtgtacatccaatccttatccctggatcattgcactcctcagagtctacatgacccggctggttggtgtctccactacgacacaattctatgctgcagacccacacccaaaagcaggcctgtcgcacctcattgaacacggtcacctccctccaaagcacaggatgccgcctgaatagcaatacttctcctctcattggaccccggctgcaattggctttaccgtttgatctgtaacgttgttcagggaaccgtggcgcaggtggtctgcaacctagatgattgctatctggccatatttgggcggcggatccgtatataaagcggagtgtagctgtagtcagcccccacgatcaatccaccatgtccaaagaactctacgccaactccaaggacgacgctgtctacttctggcgcccacgtgggtctctacgggggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaagaatgagagactttaaacatatgaatactcgccgtcagcaggcccaaaacatccgcaacaacgccgctgagctggccgctaatcgacctcaccctcagcacatcaacaacaaggaagagtacgagtacagacgaccaaagaaggacggcaacgagcccagccacattgctaacttcaccaagggcttgccccacgacgagcatacaggcctgctgctcaactccgccgactatgaccagttcgtcctgggcattcagtctggagacacgacggactttgctcgaaccccactcggacccgccgagctgcctaaggttcatggctgtctgagcaagcagaagattgactgtgatgacgaccaccgatctggattctggaagtcacagatcgctcaaggagctgcgggtggtgacggagccaagcttagggcttgggagagtgcgggggctggtctggtttttgacctcgaaggccccgacgcccaggcagtaacgatgccccccgccccgagactggagtcacctgagcttacttcagagatcgcagaggtttattcccaggccttgctccgggacatccacttctctcagctgcgagatcctggcttgggagaccaggtgaacgcctgtgattcttgcccaacccagctttctatttacgaggccattgatattctcaatactgtccagatcgagggccaaaactggttctctgccaactgctgcgatctcacagacgacgagcaggcacgacagcgacccctcgtaactcgacagaatatctttcggggcatcgctcctggagacgatgtcggtccctacctatcccagtttctgctgattggtaataacgcgctgggaggtggggtctttggccaggaagccggccatatcggatacggtgcaatacgaattgaccagcgagtcagaaaggccaccccctgcaaggacttcatgactaacttcgagacatggctggacgtgcagaacggtgcagatttgcgaggtctcgaaacctacgtggacgctgatccgggaaaatgtcgtgagtttcccgcctaccgagtcatcaccacgccccgggacctagccacctacgttcactacgatgcgctatatgaagcttatctgaacgcttgcctgattttgctgggaatgggagctcctttcgaccccggcattccgttccaaaagcccgacgtggaggacaaacagcagggctttgcccactttggaggaccgcaaatcttaaccctagtgtgtgaggccgccaccagaggccttaaagcagttcgcttccagaagttcaacgtgcaccgtagactgcgacctgaggctctcggtggtttggttgatcggtataagcacggtaagggtgcgggtgatgaacttaagcccgtcgcagccctggtcgaagcgctcgagaacgttggactcctctcgaaggtggtggcccataaccaactccagaaccagaaccttgaccgttccggcgacccctcctcggctggggataattacttcctcccaatggccttcccagaaggatcgcctatgcaccctagttacggtgccggacatgcaactgtggctggagcctgtgtcactatgcttaaagctttctttgatcatggttggcagctgaatttgggcatggcgaacggcaaatacatctcctacgagcctaaccaggacgggtcgtcgcttcagcaagtgcttctggattgccctctcacagtggagggagagttgaacaagattgctgctaacatctctattggacgggattgggctggcgtgcattacttcaccgactacatcgagtccctgcgactgggcgaaaagattgccatcggcattctggaagagcaaaagctgacctacggagagaactttacaatgaccgtccccctctacgatggcggctccattcaaatctctaagctgtaagaattcaccggttgtctgtggtatctaagctatttatcactctttacaacttctacctcaactatctactttaataaatgaatatcgtttattctctatgattactgtatatgcgttcctctaagacatctagactcctttggagttccttacccccaccatccctactcatctcagcgggccggccctaccggccctctgctgctccaggacacctatctgattgacgctctggcccatttcgaccgagagcgaatccccgagcgagtcgtccatgccaacggaggaggagcccatggctactttgaggtgaccgatgacatctccgacatcacatacgccgagcccttccagaagattggctacaaatgccccaccaccgtgcgattttctaccgttggtggtgagcgaggctctcccgacactgctcgagacccccgaggtttcgctgtcaagcacaagaccgactggggtaactgggacatggttggtctcaactctcctgttttcttcattcgagatcctgtcaagttcatccatgtcaaccactctcagaagcgagacccccagaccaacctgactgccggagatgatgcctctaactactggaactatctggtccagaaccccgagtctctgcatcaggtggtgtacatgtttggagaccgaggtacccccaatggatggcgacacatgaactgcttctccactcatacttacaagatgatcaacaaggagggcaagctgacctacgtccagttccactacaagtccgaccagggagtcaagaacttcaccggacccgaggctgctgagatggctggaaagagcccggaccacgaccagaaggatctcttctttgccatcgataacggagactacccctcctggaccgtttcccttcagaccatgactcccgagcaggccgaggaatgggaatactccattctggatatgaccaagacctggccctatgacaagttccctctgcgaaaggtcggaaagctggtgctcaacaagaacgccgagaacttctttgaggagattgagcaggccgccttctctccctccaacctgatccacggtatcgaagcctctgatgatcctgttaattaagctagcgtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatc

SEQ ID NO: 59 - DNA, 8540 bp, Artificial Sequence
  Nucleotide sequence encoding pYLHR34749gRNA2
ctattgaggaggccaagcgataccagaaggctggctgggaggcttaccagaagattaactgttagaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacaccaaaaccatgatcaaaccacccatggacttcctggaggcagaagaacttgttatggaaaagctcaagagagagaagccaagatactatcaagacatgtgtcgcaacttcaaggaggaccaagctctgtacaccgagaaacaggcctttgtcgacgatctggccccgcttttcgtagataatggaatacaaatggatatccagagtatacacatggatagtatacactgacacgacaattctgtatctctttatgttaactactgtgaggcgttaaatagagcttgatatataaaatgttacatttcacagtctgaacttttgcagattacctaatttggtaagatattaattatgaactgaaagttgatggcatccctaaatttgatgaaagttttcgagaaaatgagaacaactcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccattgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaaggccggcctgcgcaagtgttttttctatccaaaagggggccaattgcgcgtgtttcaaagagtggcgcttcagttaccttgcatcgcgatttccttgcggcgtcagagactcatcttcgtgtttgcgacatcgatatgcaagctcctacaaccccagatcttccagtccgagagagccacaaagtttgaagggggaggggtgtgacaagcccaagaagggtgacatttattttccgtggggtgtgtctttgtctacgctgtagttttcgtgatctcattcaatgatttctgtatgataatctgttgggccatcttcagaatgccttgcataatagcaaccaacattgtacctataggtggttgctgcttcatacgagcttgccgtcttatattgagtctaaagggcgaacacactgagaaacaaaaaaaaagagaacggaatccggatctatcagcccacatgcttccagacaaagagtcaaaagacctactgtattaagaccctgaccaatagagtgacctctgtgggtctcttttttgtctggcctggcccataaaagggagcctaatactggccaatctacacctgtagtcagcagtctagatttcctcttcaaggtcttttactaagagttaacaacggttctacacggcgcatgcaccaacgtatgataatgtaacatcatcacaacatccaacgaggtcgaaaccactcacacccagcactttaaacagcacccagaaatcaatatatcgtcttgagtcgctccgcggaggggaattctacctttggattgtttcataagcacgtataatcgtcccctcattggtaaataatctgactaggtttcggctaggtttggcttgcctgcatcccaggtgcagctgctgactattcgagtacctctagtgccttgctgtgagacaaggattagtaaaagtggagatgatatataaggagggaacaggacccttcgtccctggctcaattgaacagtaacatctacaaacaacatgtccaaagaactctacgccaattccaaagacgacgccgtctactccacctccttcggagtccccttcccccaccacccttggcgcccacgtgggtctctacgggggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaagaatgagagactttaaacatatgaatactcgccgtcagcaggcccaaaacatccgcaacaacgccgctgagctggccgctaatcgacctcaccctcagcacatcaacaacaaggaagagtacgagtacagacgaccaaagaaggacggcaacgagcccagccacattgctaacttcaccaagggcttgccccacgacgagcatacaggcctgctgctcaactccgccgactatgaccagttcgtcctgggcattcagtctggagacacgacggactttgctcgaaccccactcggacccgccgagctgcctaaggttcatggctgtctgagcaagcagaagattgactgtgatgacgaccaccgatctggattctggaagtcacagatcgctcaaggagctgcgggtggtgacggagccaagcttagggcttgggagagtgcgggggctggtctggtttttgacctcgaaggccccgacgcccaggcagtaacgatgccccccgccccgagactggagtcacctgagcttacttcagagatcgcagaggtttattcccaggccttgctccgggacatccacttctctcagctgcgagatcctggcttgggagaccaggtgaacgcctgtgattcttgcccaacccagctttctatttacgaggccattgatattctcaatactgtccagatcgagggccaaaactggttctctgccaactgctgcgatctcacagacgacgagcaggcacgacagcgacccctcgtaactcgacagaatatctttcggggcatcgctcctggagacgatgtcggtccctacctatcccagtttctgctgattggtaataacgcgctgggaggtggggtctttggccaggaagccggccatatcggatacggtgcaatacgaattgaccagcgagtcagaaaggccaccccctgcaaggacttcatgactaacttcgagacatggctggacgtgcagaacggtgcagatttgcgaggtctcgaaacctacgtggacgctgatccgggaaaatgtcgtgagtttcccgcctaccgagtcatcaccacgccccgggacctagccacctacgttcactacgatgcgctatatgaagcttatctgaacgcttgcctgattttgctgggaatgggagctcctttcgaccccggcattccgttccaaaagcccgacgtggaggacaaacagcagggctttgcccactttggaggaccgcaaatcttaaccctagtgtgtgaggccgccaccagaggccttaaagcagttcgcttccagaagttcaacgtgcaccgtagactgcgacctgaggctctcggtggtttggttgatcggtataagcacggtaagggtgcgggtgatgaacttaagcccgtcgcagccctggtcgaagcgctcgagaacgttggactcctctcgaaggtggtggcccataaccaactccagaaccagaaccttgaccgttccggcgacccctcctcggctggggataattacttcctcccaatggccttcccagaaggatcgcctatgcaccctagttacggtgccggacatgcaactgtggctggagcctgtgtcactatgcttaaagctttctttgatcatggttggcagctgaatttgggcatggcgaacggcaaatacatctcctacgagcctaaccaggacgggtcgtcgcttcagcaagtgcttctggattgccctctcacagtggagggagagttgaacaagattgctgctaacatctctattggacgggattgggctggcgtgcattacttcaccgactacatcgagtccctgcgactgggcgaaaagattgccatcggcattctggaagagcaaaagctgacctacggagagaactttacaatgaccgtccccctctacgatggcggctccattcaaatctctaagctgtaagaattcaccggttgtctgtggtatctaagctatttatcactctttacaacttctacctcaactatctactttaataaatgaatatcgtttattctctatgattactgtatatgcgttcctctaagacatctagaggtaccctctgctcagcgagctggtcctaccgggcctttgctgctccaggacacctatctaattgacgctctggcccatttcgaccgagagcgaattcccgagcgagttgtccatgcaaagggaggaggagcccacggctacttcgaggtcaccgatgatctgtctgacatcacctacgctgagcccttccagaaggccggctacaagtgtcccgtgacggtacgattctcaactgttggaggtgagcgaggctctgctgactcagctcgagacccccgaggcttctccatcaagcatcgaaccgactggggtaactgggacatggtggccaacaacacccccatcttcttcatccgagaccccatcaagttcccccacttcatccacacccagaagcgagatcctcagaccaacctgggtgctggaccggacgcttctcacttctggaactacctgacccagaaccccgagtctctccaccagatcacctacatgtttggagatcgaggcactcctgatggatggagacacatgaacggctactctggccacaccttcaagttcatcagcaaggagggtaagatcacctacactcagttccactacatcaccgaccagggtgtcaagaacctgtctgccgagaaggccggtgagcttgcaggtgccaaccctgattcctctcagcaggacctgtttgaggccatcgacaacggggattacccctcgtggacggtctacgtgcagaccatgactcccgagcaggccgaaaagttccggtactccattctggatcttaccaaggtttggtctcacaaggaattccctctgcgaaaggtcggcaagtttgtgctcaacaagaacgccgacaactactttgaggagattgagcaggtagcattctctccttccaacctgcccaacggtctggaggcctctgctgaccctgttctgcagtctcgactcttctcctaccccgatactcatcgacaccgactgggtcccaacttcaaccagttgccttaattaagctagcgtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatc

SEQ ID NO: 60 - DNA, 8541 bp, Artificial Sequence
  Nucleotide sequence encoding pYLHR34749RNA1
ctattgaggaggccaagcgataccagaaggctggctgggaggcttaccagaagattaactgttagaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacaccaaaaccatgatcaaaccacccatggacttcctggaggcagaagaacttgttatggaaaagctcaagagagagaagccaagatactatcaagacatgtgtcgcaacttcaaggaggaccaagctctgtacaccgagaaacaggcctttgtcgacgatctggccccgcttttcgtagataatggaatacaaatggatatccagagtatacacatggatagtatacactgacacgacaattctgtatctctttatgttaactactgtgaggcgttaaatagagcttgatatataaaatgttacatttcacagtctgaacttttgcagattacctaatttggtaagatattaattatgaactgaaagttgatggcatccctaaatttgatgaaagttttcgagaaaatgagaacaactcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccattgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaaggccggcctgcgcaagtgttttttctatccaaaagggggccaattgcgcgtgtttcaaagagtggcgcttcagttaccttgcatcgcgatttccttgcggcgtcagagactcatcttcgtgtttgcgacatcgatatgcaagctcctacaaccccagatcttccagtccgagagagccacaaagtttgaagggggaggggtgtgacaagcccaagaagggtgacatttattttccgtggggtgtgtctttgtctacgctgtagttttcgtgatctcattcaatgatttctgtatgataatctgttgggccatcttcagaatgccttgcataatagcaaccaacattgtacctataggtggttgctgcttcatacgagcttgccgtcttatattgagtctaaagggcgaacacactgagaaacaaaaaaaaagagaacggaatccggatctatcagcccacatgcttccagacaaagagtcaaaagacctactgtattaagaccctgaccaatagagtgacctctgtgggtctcttttttgtctggcctggcccataaaagggagcctaatactggccaatctacacctgtagtcagcagtctagatttcctcttcaaggtcttttactaagagttaacaacggttctacacggcgcatgcaccaacgtatgataatgtaacatcatcacaacatccaacgaggtcgaaaccactcacacccagcactttaaacagcacccagaaatcaatatatcgtcttgagtcgctccgcggaggggaattctacctttggattgtttcataagcacgtataatcgtcccctcattggtaaataatctgactaggtttcggctaggtttggcttgcctgcatcccaggtgcagctgctgactattcgagtacctctagtgccttgctgtgagacaaggattagtaaaagtggagatgatatataaggagggaacaggacccttcgtccctggctcaattgaacagtaacatctacaaacaacatgtccaaagaactctacgccaattccaaagacgacgccgtctactccacctcttggcgcccacgtgggtctctacgggggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaagaatgagagactttaaacatatgaatactcgccgtcagcaggcccaaaacatccgcaacaacgccgctgagctggccgctaatcgacctcaccctcagcacatcaacaacaaggaagagtacgagtacagacgaccaaagaaggacggcaacgagcccagccacattgctaacttcaccaagggcttgccccacgacgagcatacaggcctgctgctcaactccgccgactatgaccagttcgtcctgggcattcagtctggagacacgacggactttgctcgaaccccactcggacccgccgagctgcctaaggttcatggctgtctgagcaagcagaagattgactgtgatgacgaccaccgatctggattctggaagtcacagatcgctcaaggagctgcgggtggtgacggagccaagcttagggcttgggagagtgcgggggctggtctggtttttgacctcgaaggccccgacgcccaggcagtaacgatgccccccgccccgagactggagtcacctgagcttacttcagagatcgcagaggtttattcccaggccttgctccgggacatccacttctctcagctgcgagatcctggcttgggagaccaggtgaacgcctgtgattcttgcccaacccagctttctatttacgaggccattgatattctcaatactgtccagatcgagggccaaaactggttctctgccaactgctgcgatctcacagacgacgagcaggcacgacagcgacccctcgtaactcgacagaatatctttcggggcatcgctcctggagacgatgtcggtccctacctatcccagtttctgctgattggtaataacgcgctgggaggtggggtctttggccaggaagccggccatatcggatacggtgcaatacgaattgaccagcgagtcagaaaggccaccccctgcaaggacttcatgactaacttcgagacatggctggacgtgcagaacggtgcagatttgcgaggtctcgaaacctacgtggacgctgatccgggaaaatgtcgtgagtttcccgcctaccgagtcatcaccacgccccgggacctagccacctacgttcactacgatgcgctatatgaagcttatctgaacgcttgcctgattttgctgggaatgggagctcctttcgaccccggcattccgttccaaaagcccgacgtggaggacaaacagcagggctttgcccactttggaggaccgcaaatcttaaccctagtgtgtgaggccgccaccagaggccttaaagcagttcgcttccagaagttcaacgtgcaccgtagactgcgacctgaggctctcggtggtttggttgatcggtataagcacggtaagggtgcgggtgatgaacttaagcccgtcgcagccctggtcgaagcgctcgagaacgttggactcctctcgaaggtggtggcccataaccaactccagaaccagaaccttgaccgttccggcgacccctcctcggctggggataattacttcctcccaatggccttcccagaaggatcgcctatgcaccctagttacggtgccggacatgcaactgtggctggagcctgtgtcactatgcttaaagctttctttgatcatggttggcagctgaatttgggcatggcgaacggcaaatacatctcctacgagcctaaccaggacgggtcgtcgcttcagcaagtgcttctggattgccctctcacagtggagggagagttgaacaagattgctgctaacatctctattggacgggattgggctggcgtgcattacttcaccgactacatcgagtccctgcgactgggcgaaaagattgccatcggcattctggaagagcaaaagctgacctacggagagaactttacaatgaccgtccccctctacgatggcggctccattcaaatctctaagctgtaagaattcaccggttgtctgtggtatctaagctatttatcactctttacaacttctacctcaactatctactttaataaatgaatatcgtttattctctatgattactgtatatgcgttcctctaagacatctagaggtacctcggagtccccttcccccaccacccctactctgctcagcgagctggtcctaccgggcctttgctgctccaggacacctatctaattgacgctctggcccatttcgaccgagagcgaattcccgagcgagttgtccatgcaaagggaggaggagcccacggctacttcgaggtcaccgatgatctgtctgacatcacctacgctgagcccttccagaaggccggctacaagtgtcccgtgacggtacgattctcaactgttggaggtgagcgaggctctgctgactcagctcgagacccccgaggcttctccatcaagcatcgaaccgactggggtaactgggacatggtggccaacaacacccccatcttcttcatccgagaccccatcaagttcccccacttcatccacacccagaagcgagatcctcagaccaacctgggtgctggaccggacgcttctcacttctggaactacctgacccagaaccccgagtctctccaccagatcacctacatgtttggagatcgaggcactcctgatggatggagacacatgaacggctactctggccacaccttcaagttcatcagcaaggagggtaagatcacctacactcagttccactacatcaccgaccagggtgtcaagaacctgtctgccgagaaggccggtgagcttgcaggtgccaaccctgattcctctcagcaggacctgtttgaggccatcgacaacggggattacccctcgtggacggtctacgtgcagaccatgactcccgagcaggccgaaaagttccggtactccattctggatcttaccaaggtttggtctcacaaggaattccctctgcgaaaggtcggcaagtttgtgctcaacaagaacgccgacaactactttgaggagattgagcaggtagcattctctccttccaacctgcccaacggtctggaggcctctgctgaccctgttctgcagtctcgactcttctcctaccccgatactcatcgacaccgactgggtcccaacttcaaccagttgccttaattaagctagcgtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatc

SEQ ID NO: 61 - DNA, 8185 bp, Artificial Sequence
  Nucleotide sequence encoding pYLHR-CTA::AmVHPOSKL
gagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacaccaaaaccatgatcaaaccacccatggacttcctggaggcagaagaacttgttatggaaaagctcaagagagagaagccaagatactatcaagacatgtgtcgcaacttcaaggaggaccaagctctgtacaccgagaaacaggcctttgtcgacgatctggccccgcttttcgtagataatggaatacaaatggatatccagagtatacacatggatagtatacactgacacgacaattctgtatctctttatgttaactactgtgaggcgttaaatagagcttgatatataaaatgttacatttcacagtctgaacttttgcagattacctaatttggtaagatattaattatgaactgaaagttgatggcatccctaaatttgatgaaagttttcgagaaaatgagaacaactcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaaggccggcctgcgcacgtaatagagttcaagcacaagaacacggctggggcttccagtagtggcaatatgcgtgtgttttggcggttggaaatgtccgatatggccctcaacgactggcagacagataagacaaggccgcggagaagttcactgaaacaaagcgtgtaaaacacagcgttggaatggcacaaacagcagaccttctacaggctctggaaacagaagaaaacctatttctaccctttccttctctcaccctacttgtactttattgcgcgtggggaaggttcaggctgttttctgttttctgggcttttgacgtttggaccgcggctcaaagacgagaaaagggacgtccgtcctcgctctaacacgttgaaacgcttttttcttagtctacattgacatgcttgctgtctcatgcattcggcacaaagaatctgtaggaagtaacgattggggggaaaccagcttcaacaagacacgagtgagttcgcggaccctttctccaccgctggattatcttatttgcagctgtaacaggttgtcgtgggtagttctgtctgtctcaggctgtagatatagtcgatcatgacaaacgctgcaaggctattggctgtgaccccgggttttcccctcccaacaagaacttagttaaggtgataatgaagagggcatggtaggtgccgaaagaagcaccgagtggcgagtccgagagagtagctacgtggaaatcaaagaattgaatggggaaaagtgctcgatcgaaaaatccagctagttttgagctcgttagagccccaaaacagccataaccatttctcaactccacaccgccatctcgcccttaacccaaatctagtccacacatttccccacgataactctatattatgcggtggcttcggtaattctctttgtttgcgactttcacacgcccctttaagctcccacaaaccatgtgcgtgtgattatatatatataggtgggggtagcgaaacgccgagtcaagggcgcccatatgaatactcgccgtcagcaggcccaaaacatccgcaacaacgccgctgagctggccgctaatcgacctcaccctcagcacatcaacaacaaggaagagtacgagtacagacgaccaaagaaggacggcaacgagcccagccacattgctaacttcaccaagggcttgccccacgacgagcatacaggcctgctgctcaactccgccgactatgaccagttcgtcctgggcattcagtctggagacacgacggactttgctcgaaccccactcggacccgccgagctgcctaaggttcatggctgtctgagcaagcagaagattgactgtgatgacgaccaccgatctggattctggaagtcacagatcgctcaaggagctgcgggtggtgacggagccaagcttagggcttgggagagtgcgggggctggtctggtttttgacctcgaaggccccgacgcccaggcagtaacgatgccccccgccccgagactggagtcacctgagcttacttcagagatcgcagaggtttattcccaggccttgctccgggacatccacttctctcagctgcgagatcctggcttgggagaccaggtgaacgcctgtgattcttgcccaacccagctttctatttacgaggccattgatattctcaatactgtccagatcgagggccaaaactggttctctgccaactgctgcgatctcacagacgacgagcaggcacgacagcgacccctcgtaactcgacagaatatctttcggggcatcgctcctggagacgatgtcggtccctacctatcccagtttctgctgattggtaataacgcgctgggaggtggggtctttggccaggaagccggccatatcggatacggtgcaatacgaattgaccagcgagtcagaaaggccaccccctgcaaggacttcatgactaacttcgagacatggctggacgtgcagaacggtgcagatttgcgaggtctcgaaacctacgtggacgctgatccgggaaaatgtcgtgagtttcccgcctaccgagtcatcaccacgccccgggacctagccacctacgttcactacgatgcgctatatgaagcttatctgaacgcttgcctgattttgctgggaatgggagctcctttcgaccccggcattccgttccaaaagcccgacgtggaggacaaacagcagggctttgcccactttggaggaccgcaaatcttaaccctagtgtgtgaggccgccaccagaggccttaaagcagttcgcttccagaagttcaacgtgcaccgtagactgcgacctgaggctctcggtggtttggttgatcggtataagcacggtaagggtgcgggtgatgaacttaagcccgtcgcagccctggtcgaagcgctcgagaacgttggactcctctcgaaggtggtggcccataaccaactccagaaccagaaccttgaccgttccggcgacccctcctcggctggggataattacttcctcccaatggccttcccagaaggatcgcctatgcaccctagttacggtgccggacatgcaactgtggctggagcctgtgtcactatgcttaaagctttctttgatcatggttggcagctgaatttgggcatggcgaacggcaaatacatctcctacgagcctaaccaggacgggtcgtcgcttcagcaagtgcttctggattgccctctcacagtggagggagagttgaacaagattgctgctaacatctctattggacgggattgggctggcgtgcattacttcaccgactacatcgagtccctgcgactgggcgaaaagattgccatcggcattctggaagagcaaaagctgacctacggagagaactttacaatgaccgtccccctctacgatggcggctccattcaaatctctaaactctaagaattcgtcgactcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcttaattaatctagaaactaaggccttattgaaaagtgagtacccaggaggactgtttggacgggcaattcgataccgggaaagcagccccatggacgcagcgggttgattcggatgatctggtggtttcacgggctatagatattgccccatcaatgaggggaagacgctcaagtccgaacaccgcaacataatacgacagtggtgatagacaactcgcctaaagccaacaacaacaacaccaatagcatactaactcagtgtctggaaaacgagtccccacttacaccaccactaacggtaaccccgtcaaggaccccctcgcctcccagcgaatcggcaagcacggacctctcctgctccaggactttgccctgatcgacctgctcgcccacttcgaccgagagcgaatccccgagcgagttgtccacgctaagggatccggtgcttacggagagttcgaggtcactgacgatatcaccgacctcaactgcgccgatttcttgtccaagatcggtaagaagaccaagaccttcactcgattctccaccgttggaggagagaagggatccgccgatgctgctcgagacccccgaggtttcgccaccaagttctacaccgacgagggaaacattgactgggtctacaacaacacccccgtctttttcatcagagacccctccaagttccccgttttcatccacactcagaagcgaaaccccgagaccaacctcaaggacgccactatgatgtgggactacattgccaacaaccaggagtgttgccaccagatcatggtcctcttctccgaccgaggtactcccgctaactaccgacagatgaacggttactccggccacacctacaagtggatcaagaaggatggctctttcaactacgtccagatccacatgaagaccgaccagggcatcaagaacctcaccaatgacgaggctgtcgctctctccggaaccaaccccgaccacgcccaggaagacctcttcaactccatcaagagtgggtaccgcggccgc